SEQUENCE LISTING (47 sequences)

SEQ ID NO: 1 - DNA, 145 bp, unidentified
  source 1..145: mol_type = other DNA; note = Adeno-associated Virus
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 2 - DNA, 600 bp, Homo sapiens
  source 1..600: mol_type = other DNA
gtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgatcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 3 - DNA, 147 bp, synthetic construct
  source 1..147: mol_type = other DNA
caggtaagtatcaaggttacaagacaggtttaaggagaccaatagaaactgggcttgtcgagacagagggccggccaagactcttgcgtttctgataggcacctattggtcttactgacatccactttgcctttctctccacagggt

SEQ ID NO: 4 - skipped

SEQ ID NO: 5 - DNA, 3684 bp, synthetic construct
  source 1..3684: mol_type = other DNA
atggtgctggcagcagccatgagccaggacgcggaccccagcggtccggagcagccggacagagttgcctgcagtgtgcctggtgcccgggcgtccccggcaccctccggcccgcgagggatgcagcagccgccgccgccgccccagccaccgcccccgccccaagccggcctaccccagatcatccaaaatgccgccaagctcctggacaagaacccattctcggtcagtaacccgaaccctctgcttccttcacctgccagtctccagctggctcaactgcaggcccagctcaccctccaccggctgaagctggcacagacagctgtcaccaacaacactgcagccgccacagtcctgaaccaagtcctctccaaagtggccatgtcccagcctctcttcaatcaactgaggcatccgtctgtgatcactggcccccacggccatgctggggttccccaacatgctgcagccatacccagtacccggtttccctctaatgcaattgccttttcaccccccagccagacacgaggccccggaccctccatgaaccttcccaaccagccacccagtgccatggtgatgcatcctttcactggggtaatgcctcagacccctggccagccagcagtcatcttgggcattggcaagactgggcctgctccagctacagcaggattctatgagtatggcaaagccagctctggccagacatatggccctgaaacagatggtcagcctggcttcctgccatcctcggcctcaacctcgggcagtgtgacctatgaagggcactacagccacacagggcaggatggtcaagctgccttttccaaagatttttacggacccaactcccaaggttcacatgtggccagcggatttccagctgagcaggctgggggcctgaaaagtgaggtcgggccactgctgcagggcacaaacagccaatgggagagcccccatggattctcgggccaaagcaagcctgatctcacagcaggtcccatgtggcctccaccccacaaccagccctatgagctgtacgaccccgaggaaccaacctcagacaggacacctccttccttcgggggtcggcttaacaacagcaaacagggttttatcggtgctgggcggagggccaaggaggaccaggcgttgctatctgtgcggcctctgcaggctcatgagctgaacgactttcacggtgtggcccccctccacttgccgcatatctgtagcatctgtgacaagaaggtgtttgatttgaaggactgggagctgcatgtgaaagggaagctgcacgctcagaaatgcctggtcttctctgaaaatgctggcatccggtgtatacttggttcggcagagggaacattgtgtgcttctcccaacagcacagctgtttataaccctgctgggaatgaagattatgcctcaaatcttggaacatcatacgtgcccattccagcaaggtcattcactcagtcaagccccacatttcctttggcttctgtggggacaacttttgcacagcggaaaggggctggccgtgtggtgcacatctgcaatctccctgaaggaagctgcactgagaatgacgtcattaacctggggctgccctttggaaaggtcactaattacatcctcatgaaatcgactaatcaggcctttttagagatggcttacacagaagctgcacaggccatggtccagtattatcaagaaaaatctgctgtgatcaatggtgagaagttgctcattcggatgtccaagagatacaaggaattgcagctcaagaaacccgggaaggccgtggctgccatcatccaggacatccattcccagagggagagggacatgttccgggaagcagacagatatggcccagaaaggccgcggtctcgtagtccggtgagccggtcactctccccgaggtcccacactcccagcttcacctcctgcagctcttcccacagccctccgggcccctcccgggctgactggggcaatggccgggactcctgggagcactctccctatgccaggagggaggaagagcgagacccggctccctggagggacaacggagatgacaagagggacaggatggacccctgggcacatgatcgcaaacaccacccccggcaactggacaaggctgagttggacgagcgaccagaaggagggaggccccaccgggagaagtacccgagatctgggtctcccaacctgccccactctgtgtccagctacaaaagccgtgaagacggctactaccggaaagagcccaaagccaagtgggacaagtatctgaagcagcagcaggatgcccccgggaggtccaggaggaaagacgaggccaggctgcgggaaagcagacacccccatccggatgactcaggcaaggaagatgggctggggccaaaggtcactagggcccctgagggcgccaaggccaagcagaatgagaaaaataaaaccaagagaactgatagagaccaagaaggagctgatgatagaaaagaaaacacaatggcagagaatgaggctggaaaagaggaacaggagggcatggaagaaagccctcaatcagtgggcagacaggagaaagaagcagagttctctgatccggaaaacacaaggacaaagaaggaacaagattgggagagtgaaagtgaggcagagggggagagctggtatcccactaacatggaggagctggtgacagtggacgaggttggggaagaagaagattttatcgtggaaccagacatcccagagctggaagaaattgtgcccattgaccagaaagacaaaatttgcccagaaacatgtctgtgtgtgacaaccaccttagacttagacctggcccaggatttccccaaggaaggagtcaaggccgtagggaatggggctgcagaaatcagcctcaagtcacccagagaactgccctctgcttccacaagctgtcccagtgacatggacgtggaaatgcctggcctaaatctggatgctgagcggaagccagctgaaagtgagacaggcctctccctggaggattcagattgctacgagaaggaggcaaagggagtggagagctcagatgttcatccagcccctacagtccagcaaatgtcttcccctaagccagcagaggagagggcccggcagccaagcccatttgtggatgattgcaagaccagggggacccccgaagatggggcttgtgaaggcagccccctggaggagaaagccagcccccccatcgaaactgacctccaaaaccaagcctgccaagaagtgttgaccccggaaaactccaggtacgtggaaatgaaatctctggaggtgaggtcaccagagtacactgaagtggaactgaaacagcccctttctttgccctcttgggaaccagaggatgtgttcagtgaacttagcattcctctaggggtggagttcgtggttcccaggactggcttttattgcaagctgtgtgggctgttctacacgagcgaggagacagcaaagatgagccactgccgcagcgctgtccactacaggaacttacagaaatatttgtcccagctggccgaggagggcctcaaggagaccgagggggcagatagcccgaggccagaggacagcggaatcgtgccacgcttcgaaaggaaaaagctctga

SEQ ID NO: 6 - DNA, 49 bp, synthetic construct
  source 1..49: mol_type = other DNA
aataaaagatccttattttcattggatctgtgtgttggttttttgtgtg

SEQ ID NO: 7 - DNA, 145 bp, unidentified
  source 1..145: mol_type = other DNA; note = Adeno-associated Virus
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 8 - protein, 1227 aa, Homo sapiens
MVLAAAMSQDADPSGPEQPDRVACSVPGARASPAPSGPRGMQQPPPPPQPPPPPQAGLPQIIQNAAKLLDKNPFSVSNPNPLLPSPASLQLAQLQAQLTLHRLKLAQTAVTNNTAAATVLNQVLSKVAMSQPLFNQLRHPSVITGPHGHAGVPQHAAAIPSTRFPSNAIAFSPPSQTRGPGPSMNLPNQPPSAMVMHPFTGVMPQTPGQPAVILGIGKTGPAPATAGFYEYGKASSGQTYGPETDGQPGFLPSSASTSGSVTYEGHYSHTGQDGQAAFSKDFYGPNSQGSHVASGFPAEQAGGLKSEVGPLLQGTNSQWESPHGFSGQSKPDLTAGPMWPPPHNQPYELYDPEEPTSDRTPPSFGGRLNNSKQGFIGAGRRAKEDQALLSVRPLQAHELNDFHGVAPLHLPHICSICDKKVFDLKDWELHVKGKLHAQKCLVFSENAGIRCILGSAEGTLCASPNSTAVYNPAGNEDYASNLGTSYVPIPARSFTQSSPTFPLASVGTTFAQRKGAGRVVHICNLPEGSCTENDVINLGLPFGKVTNYILMKSTNQAFLEMAYTEAAQAMVQYYQEKSAVINGEKLLIRMSKRYKELQLKKPGKAVAAIIQDIHSQRERDMFREADRYGPERPRSRSPVSRSLSPRSHTPSFTSCSSSHSPPGPSRADWGNGRDSWEHSPYARREEERDPAPWRDNGDDKRDRMDPWAHDRKHHPRQLDKAELDERPEGGRPHREKYPRSGSPNLPHSVSSYKSREDGYYRKEPKAKWDKYLKQQQDAPGRSRRKDEARLRESRHPHPDDSGKEDGLGPKVTRAPEGAKAKQNEKNKTKRTDRDQEGADDRKENTMAENEAGKEEQEGMEESPQSVGRQEKEAEFSDPENTRTKKEQDWESESEAEGESWYPTNMEELVTVDEVGEEEDFIVEPDIPELEEIVPIDQKDKICPETCLCVTTTLDLDLAQDFPKEGVKAVGNGAAEISLKSPRELPSASTSCPSDMDVEMPGLNLDAERKPAESETGLSLEDSDCYEKEAKGVESSDVHPAPTVQQMSSPKPAEERARQPSPFVDDCKTRGTPEDGACEGSPLEEKASPPIETDLQNQACQEVLTPENSRYVEMKSLEVRSPEYTEVELKQPLSLPSWEPEDVFSELSIPLGVEFVVPRTGFYCKLCGLFYTSEETAKMSHCRSAVHYRNLQKYLSQLAEEGLKETEGADSPRPEDSGIVPRFERKKL

SEQ ID NO: 9 - DNA, 191 bp, synthetic construct
  source 1..191: mol_type = other DNA
ccttcagattaaaaataactaaggtaagggccatgtgggtaggggaggtggtgtgagacggtcctgtctctcctctatctgcccatcggccctttggggaggaggaatgtgcccaaggactaaaaaaaggccctggagccagaggggcgagggcagcagacctttcatgggcaaacctcagggctgctgtc

SEQ ID NO: 10 - DNA, 2217 bp, unidentified
  source 1..2217: mol_type = other DNA; note = Adeno-associated Virus serotype rh74
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 11 - protein, 738 aa, unidentified
  source 1..738: note = Adeno-associated Virus serotype rh74
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 12 - protein, 735 aa, unidentified
  source 1..735: note = Adeno-associated Virus serotype 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 13 - DNA, 145 bp, unidentified; identical to SEQ ID NO: 1
  source 1..145: mol_type = other DNA; note = Adeno-associated Virus

SEQ ID NO: 14 - DNA, 191 bp, Homo sapiens
  source 1..191: mol_type = other DNA
acccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgt

SEQ ID NO: 15 - DNA, 111 bp, Homo sapiens
  source 1..111: mol_type = other DNA
ctgcccatgtaaggaggcaaggcctggggacacccgagatgcctggttataattaacccagacatgtggctgcccccccccccccaacacctgctgcctctaaaaataacc

SEQ ID NO: 16 - DNA, 265 bp, Homo sapiens
  source 1..265: mol_type = other DNA
gttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctc

SEQ ID NO: 17 - DNA, 43 bp, Homo sapiens
  source 1..43: mol_type = other DNA
accaccacctccacagcacagacagacactcaggagcagccag

SEQ ID NO: 18 - DNA, 147 bp, synthetic construct; identical to SEQ ID NO: 3
  source 1..147: mol_type = other DNA

SEQ ID NO: 19 - DNA, 3684 bp, synthetic construct; identical to SEQ ID NO: 5
  source 1..3684: mol_type = other DNA

SEQ ID NO: 20 - DNA, 49 bp, synthetic construct; identical to SEQ ID NO: 6
  source 1..49: mol_type = other DNA

SEQ ID NO: 21 - DNA, 145 bp, unidentified; identical to SEQ ID NO: 7
  source 1..145: mol_type = other DNA; note = Adeno-associated Virus

SEQ ID NO: 22-37 - skipped (16 entries)

SEQ ID NO: 38 - DNA, 4310 bp, synthetic construct
  source 1..4310: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgagcgccaccatggtgctggcagcagccatgagccaggacgcggaccccagcggtccggagcagccggacagagttgcctgcagtgtgcctggtgcccgggcgtccccggcaccctccggcccgcgagggatgcagcagccgccgccgccgccccagccaccgcccccgccccaagccggcctaccccagatcatccaaaatgccgccaagctcctggacaagaacccattctcggtcagtaacccgaaccctctgcttccttcacctgccagtctccagctggctcaactgcaggcccagctcaccctccaccggctgaagctggcacagacagctgtcaccaacaacactgcagccgccacagtcctgaaccaagtcctctccaaagtggccatgtcccagcctctcttcaatcaactgaggcatccgtctgtgatcactggcccccacggccatgctggggttccccaacatgctgcagccatacccagtacccggtttccctctaatgcaattgccttttcaccccccagccagacacgaggccccggaccctccatgaaccttcccaaccagccacccagtgccatggtgatgcatcctttcactggggtaatgcctcagacccctggccagccagcagtcatcttgggcattggcaagactgggcctgctccagctacagcaggattctatgagtatggcaaagccagctctggccagacatatggccctgaaacagatggtcagcctggcttcctgccatcctcggcctcaacctcgggcagtgtgacctatgaagggcactacagccacacagggcaggatggtcaagctgccttttccaaagatttttacggacccaactcccaaggttcacatgtggccagcggatttccagctgagcaggctgggggcctgaaaagtgaggtcgggccactgctgcagggcacaaacagccaatgggagagcccccatggattctcgggccaaagcaagcctgatctcacagcaggtcccatgtggcctccaccccacaaccagccctatgagctgtacgaccccgaggaaccaacctcagacaggacacctccttccttcgggggtcggcttaacaacagcaaacagggttttatcggtgctgggcggagggccaaggaggaccaggcgttgctatctgtgcggcctctgcaggctcatgagctgaacgactttcacggtgtggcccccctccacttgccgcatatctgtagcatctgtgacaagaaggtgtttgatttgaaggactgggagctgcatgtgaaagggaagctgcacgctcagaaatgcctggtcttctctgaaaatgctggcatccggtgtatacttggttcggcagagggaacattgtgtgcttctcccaacagcacagctgtttataaccctgctgggaatgaagattatgcctcaaatcttggaacatcatacgtgcccattccagcaaggtcattcactcagtcaagccccacatttcctttggcttctgtggggacaacttttgcacagcggaaaggggctggccgtgtggtgcacatctgcaatctccctgaaggaagctgcactgagaatgacgtcattaacctggggctgccctttggaaaggtcactaattacatcctcatgaaatcgactaatcaggcctttttagagatggcttacacagaagctgcacaggccatggtccagtattatcaagaaaaatctgctgtgatcaatggtgagaagttgctcattcggatgtccaagagatacaaggaattgcagctcaagaaacccgggaaggccgtggctgccatcatccaggacatccattcccagagggagagggacatgttccgggaagcagacagatatggcccagaaaggccgcggtctcgtagtccggtgagccggtcactctccccgaggtcccacactcccagcttcacctcctgcagctcttcccacagccctccgggcccctcccgggctgactggggcaatggccgggactcctgggagcactctccctatgccaggagggaggaagagcgagacccggctccctggagggacaacggagatgacaagagggacaggatggacccctgggcacatgatcgcaaacaccacccccggcaactggacaaggctgagttggacgagcgaccagaaggagggaggccccaccgggagaagtacccgagatctgggtctcccaacctgccccactctgtgtccagctacaaaagccgtgaagacggctactaccggaaagagcccaaagccaagtgggacaagtatctgaagcagcagcaggatgcccccgggaggtccaggaggaaagacgaggccaggctgcgggaaagcagacacccccatccggatgactcaggcaaggaagatgggctggggccaaaggtcactagggcccctgagggcgccaaggccaagcagaatgagaaaaataaaaccaagagaactgatagagaccaagaaggagctgatgatagaaaagaaaacacaatggcagagaatgaggctggaaaagaggaacaggagggcatggaagaaagccctcaatcagtgggcagacaggagaaagaagcagagttctctgatccggaaaacacaaggacaaagaaggaacaagattgggagagtgaaagtgaggcagagggggagagctggtatcccactaacatggaggagctggtgacagtggacgaggttggggaagaagaagattttatcgtggaaccagacatcccagagctggaagaaattgtgcccattgaccagaaagacaaaatttgcccagaaacatgtctgtgtgtgacaaccaccttagacttagacctggcccaggatttccccaaggaaggagtcaaggccgtagggaatggggctgcagaaatcagcctcaagtcacccagagaactgccctctgcttccacaagctgtcccagtgacatggacgtggaaatgcctggcctaaatctggatgctgagcggaagccagctgaaagtgagacaggcctctccctggaggattcagattgctacgagaaggaggcaaagggagtggagagctcagatgttcatccagcccctacagtccagcaaatgtcttcccctaagccagcagaggagagggcccggcagccaagcccatttgtggatgattgcaagaccagggggacccccgaagatggggcttgtgaaggcagccccctggaggagaaagccagcccccccatcgaaactgacctccaaaaccaagcctgccaagaagtgttgaccccggaaaactccaggtacgtggaaatgaaatctctggaggtgaggtcaccagagtacactgaagtggaactgaaacagcccctttctttgccctcttgggaaccagaggatgtgttcagtgaacttagcattcctctaggggtggagttcgtggttcccaggactggcttttattgcaagctgtgtgggctgttctacacgagcgaggagacagcaaagatgagccactgccgcagcgctgtccactacaggaacttacagaaatatttgtcccagctggccgaggagggcctcaaggagaccgagggggcagatagcccgaggccagaggacagcggaatcgtgccacgcttcgaaaggaaaaagctctgaaataaaagatccttattttcattggatctgtgtgttggttttttgtgtgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 39 - DNA, 4310 bp, synthetic construct
  source 1..4310: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgagccccaacatggtgctggcagcagccatgagccaggacgcggaccccagcggtccggagcagccggacagagttgcctgcagtgtgcctggtgcccgggcgtccccggcaccctccggcccgcgagggatgcagcagccgccgccgccgccccagccaccgcccccgccccaagccggcctaccccagatcatccaaaatgccgccaagctcctggacaagaacccattctcggtcagtaacccgaaccctctgcttccttcacctgccagtctccagctggctcaactgcaggcccagctcaccctccaccggctgaagctggcacagacagctgtcaccaacaacactgcagccgccacagtcctgaaccaagtcctctccaaagtggccatgtcccagcctctcttcaatcaactgaggcatccgtctgtgatcactggcccccacggccatgctggggttccccaacatgctgcagccatacccagtacccggtttccctctaatgcaattgccttttcaccccccagccagacacgaggccccggaccctccatgaaccttcccaaccagccacccagtgccatggtgatgcatcctttcactggggtaatgcctcagacccctggccagccagcagtcatcttgggcattggcaagactgggcctgctccagctacagcaggattctatgagtatggcaaagccagctctggccagacatatggccctgaaacagatggtcagcctggcttcctgccatcctcggcctcaacctcgggcagtgtgacctatgaagggcactacagccacacagggcaggatggtcaagctgccttttccaaagatttttacggacccaactcccaaggttcacatgtggccagcggatttccagctgagcaggctgggggcctgaaaagtgaggtcgggccactgctgcagggcacaaacagccaatgggagagcccccatggattctcgggccaaagcaagcctgatctcacagcaggtcccatgtggcctccaccccacaaccagccctatgagctgtacgaccccgaggaaccaacctcagacaggacacctccttccttcgggggtcggcttaacaacagcaaacagggttttatcggtgctgggcggagggccaaggaggaccaggcgttgctatctgtgcggcctctgcaggctcatgagctgaacgactttcacggtgtggcccccctccacttgccgcatatctgtagcatctgtgacaagaaggtgtttgatttgaaggactgggagctgcatgtgaaagggaagctgcacgctcagaaatgcctggtcttctctgaaaatgctggcatccggtgtatacttggttcggcagagggaacattgtgtgcttctcccaacagcacagctgtttataaccctgctgggaatgaagattatgcctcaaatcttggaacatcatacgtgcccattccagcaaggtcattcactcagtcaagccccacatttcctttggcttctgtggggacaacttttgcacagcggaaaggggctggccgtgtggtgcacatctgcaatctccctgaaggaagctgcactgagaatgacgtcattaacctggggctgccctttggaaaggtcactaattacatcctcatgaaatcgactaatcaggcctttttagagatggcttacacagaagctgcacaggccatggtccagtattatcaagaaaaatctgctgtgatcaatggtgagaagttgctcattcggatgtccaagagatacaaggaattgcagctcaagaaacccgggaaggccgtggctgccatcatccaggacatccattcccagagggagagggacatgttccgggaagcagacagatatggcccagaaaggccgcggtctcgtagtccggtgagccggtcactctccccgaggtcccacactcccagcttcacctcctgcagctcttcccacagccctccgggcccctcccgggctgactggggcaatggccgggactcctgggagcactctccctatgccaggagggaggaagagcgagacccggctccctggagggacaacggagatgacaagagggacaggatggacccctgggcacatgatcgcaaacaccacccccggcaactggacaaggctgagttggacgagcgaccagaaggagggaggccccaccgggagaagtacccgagatctgggtctcccaacctgccccactctgtgtccagctacaaaagccgtgaagacggctactaccggaaagagcccaaagccaagtgggacaagtatctgaagcagcagcaggatgcccccgggaggtccaggaggaaagacgaggccaggctgcgggaaagcagacacccccatccggatgactcaggcaaggaagatgggctggggccaaaggtcactagggcccctgagggcgccaaggccaagcagaatgagaaaaataaaaccaagagaactgatagagaccaagaaggagctgatgatagaaaagaaaacacaatggcagagaatgaggctggaaaagaggaacaggagggcatggaagaaagccctcaatcagtgggcagacaggagaaagaagcagagttctctgatccggaaaacacaaggacaaagaaggaacaagattgggagagtgaaagtgaggcagagggggagagctggtatcccactaacatggaggagctggtgacagtggacgaggttggggaagaagaagattttatcgtggaaccagacatcccagagctggaagaaattgtgcccattgaccagaaagacaaaatttgcccagaaacatgtctgtgtgtgacaaccaccttagacttagacctggcccaggatttccccaaggaaggagtcaaggccgtagggaatggggctgcagaaatcagcctcaagtcacccagagaactgccctctgcttccacaagctgtcccagtgacatggacgtggaaatgcctggcctaaatctggatgctgagcggaagccagctgaaagtgagacaggcctctccctggaggattcagattgctacgagaaggaggcaaagggagtggagagctcagatgttcatccagcccctacagtccagcaaatgtcttcccctaagccagcagaggagagggcccggcagccaagcccatttgtggatgattgcaagaccagggggacccccgaagatggggcttgtgaaggcagccccctggaggagaaagccagcccccccatcgaaactgacctccaaaaccaagcctgccaagaagtgttgaccccggaaaactccaggtacgtggaaatgaaatctctggaggtgaggtcaccagagtacactgaagtggaactgaaacagcccctttctttgccctcttgggaaccagaggatgtgttcagtgaacttagcattcctctaggggtggagttcgtggttcccaggactggcttttattgcaagctgtgtgggctgttctacacgagcgaggagacagcaaagatgagccactgccgcagcgctgtccactacaggaacttacagaaatatttgtcccagctggccgaggagggcctcaaggagaccgagggggcagatagcccgaggccagaggacagcggaatcgtgccacgcttcgaaaggaaaaagctctgaaataaaagatccttattttcattggatctgtgtgttggttttttgtgtgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 40 - DNA, 4310 bp, synthetic construct
  source 1..4310: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgcaacccagcatggtgctggcagcagccatgagccaggacgcggaccccagcggtccggagcagccggacagagttgcctgcagtgtgcctggtgcccgggcgtccccggcaccctccggcccgcgagggatgcagcagccgccgccgccgccccagccaccgcccccgccccaagccggcctaccccagatcatccaaaatgccgccaagctcctggacaagaacccattctcggtcagtaacccgaaccctctgcttccttcacctgccagtctccagctggctcaactgcaggcccagctcaccctccaccggctgaagctggcacagacagctgtcaccaacaacactgcagccgccacagtcctgaaccaagtcctctccaaagtggccatgtcccagcctctcttcaatcaactgaggcatccgtctgtgatcactggcccccacggccatgctggggttccccaacatgctgcagccatacccagtacccggtttccctctaatgcaattgccttttcaccccccagccagacacgaggccccggaccctccatgaaccttcccaaccagccacccagtgccatggtgatgcatcctttcactggggtaatgcctcagacccctggccagccagcagtcatcttgggcattggcaagactgggcctgctccagctacagcaggattctatgagtatggcaaagccagctctggccagacatatggccctgaaacagatggtcagcctggcttcctgccatcctcggcctcaacctcgggcagtgtgacctatgaagggcactacagccacacagggcaggatggtcaagctgccttttccaaagatttttacggacccaactcccaaggttcacatgtggccagcggatttccagctgagcaggctgggggcctgaaaagtgaggtcgggccactgctgcagggcacaaacagccaatgggagagcccccatggattctcgggccaaagcaagcctgatctcacagcaggtcccatgtggcctccaccccacaaccagccctatgagctgtacgaccccgaggaaccaacctcagacaggacacctccttccttcgggggtcggcttaacaacagcaaacagggttttatcggtgctgggcggagggccaaggaggaccaggcgttgctatctgtgcggcctctgcaggctcatgagctgaacgactttcacggtgtggcccccctccacttgccgcatatctgtagcatctgtgacaagaaggtgtttgatttgaaggactgggagctgcatgtgaaagggaagctgcacgctcagaaatgcctggtcttctctgaaaatgctggcatccggtgtatacttggttcggcagagggaacattgtgtgcttctcccaacagcacagctgtttataaccctgctgggaatgaagattatgcctcaaatcttggaacatcatacgtgcccattccagcaaggtcattcactcagtcaagccccacatttcctttggcttctgtggggacaacttttgcacagcggaaaggggctggccgtgtggtgcacatctgcaatctccctgaaggaagctgcactgagaatgacgtcattaacctggggctgccctttggaaaggtcactaattacatcctcatgaaatcgactaatcaggcctttttagagatggcttacacagaagctgcacaggccatggtccagtattatcaagaaaaatctgctgtgatcaatggtgagaagttgctcattcggatgtccaagagatacaaggaattgcagctcaagaaacccgggaaggccgtggctgccatcatccaggacatccattcccagagggagagggacatgttccgggaagcagacagatatggcccagaaaggccgcggtctcgtagtccggtgagccggtcactctccccgaggtcccacactcccagcttcacctcctgcagctcttcccacagccctccgggcccctcccgggctgactggggcaatggccgggactcctgggagcactctccctatgccaggagggaggaagagcgagacccggctccctggagggacaacggagatgacaagagggacaggatggacccctgggcacatgatcgcaaacaccacccccggcaactggacaaggctgagttggacgagcgaccagaaggagggaggccccaccgggagaagtacccgagatctgggtctcccaacctgccccactctgtgtccagctacaaaagccgtgaagacggctactaccggaaagagcccaaagccaagtgggacaagtatctgaagcagcagcaggatgcccccgggaggtccaggaggaaagacgaggccaggctgcgggaaagcagacacccccatccggatgactcaggcaaggaagatgggctggggccaaaggtcactagggcccctgagggcgccaaggccaagcagaatgagaaaaataaaaccaagagaactgatagagaccaagaaggagctgatgatagaaaagaaaacacaatggcagagaatgaggctggaaaagaggaacaggagggcatggaagaaagccctcaatcagtgggcagacaggagaaagaagcagagttctctgatccggaaaacacaaggacaaagaaggaacaagattgggagagtgaaagtgaggcagagggggagagctggtatcccactaacatggaggagctggtgacagtggacgaggttggggaagaagaagattttatcgtggaaccagacatcccagagctggaagaaattgtgcccattgaccagaaagacaaaatttgcccagaaacatgtctgtgtgtgacaaccaccttagacttagacctggcccaggatttccccaaggaaggagtcaaggccgtagggaatggggctgcagaaatcagcctcaagtcacccagagaactgccctctgcttccacaagctgtcccagtgacatggacgtggaaatgcctggcctaaatctggatgctgagcggaagccagctgaaagtgagacaggcctctccctggaggattcagattgctacgagaaggaggcaaagggagtggagagctcagatgttcatccagcccctacagtccagcaaatgtcttcccctaagccagcagaggagagggcccggcagccaagcccatttgtggatgattgcaagaccagggggacccccgaagatggggcttgtgaaggcagccccctggaggagaaagccagcccccccatcgaaactgacctccaaaaccaagcctgccaagaagtgttgaccccggaaaactccaggtacgtggaaatgaaatctctggaggtgaggtcaccagagtacactgaagtggaactgaaacagcccctttctttgccctcttgggaaccagaggatgtgttcagtgaacttagcattcctctaggggtggagttcgtggttcccaggactggcttttattgcaagctgtgtgggctgttctacacgagcgaggagacagcaaagatgagccactgccgcagcgctgtccactacaggaacttacagaaatatttgtcccagctggccgaggagggcctcaaggagaccgagggggcagatagcccgaggccagaggacagcggaatcgtgccacgcttcgaaaggaaaaagctctgaaataaaagatccttattttcattggatctgtgtgttggttttttgtgtgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 41 - DNA, 4387 bp, synthetic construct
  source 1..4387: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcagcgccaccatggtgctggcagcagccatgagccaggacgcggaccccagcggtccggagcagccggacagagttgcctgcagtgtgcctggtgcccgggcgtccccggcaccctccggcccgcgagggatgcagcagccgccgccgccgccccagccaccgcccccgccccaagccggcctaccccagatcatccaaaatgccgccaagctcctggacaagaacccattctcggtcagtaacccgaaccctctgcttccttcacctgccagtctccagctggctcaactgcaggcccagctcaccctccaccggctgaagctggcacagacagctgtcaccaacaacactgcagccgccacagtcctgaaccaagtcctctccaaagtggccatgtcccagcctctcttcaatcaactgaggcatccgtctgtgatcactggcccccacggccatgctggggttccccaacatgctgcagccatacccagtacccggtttccctctaatgcaattgccttttcaccccccagccagacacgaggccccggaccctccatgaaccttcccaaccagccacccagtgccatggtgatgcatcctttcactggggtaatgcctcagacccctggccagccagcagtcatcttgggcattggcaagactgggcctgctccagctacagcaggattctatgagtatggcaaagccagctctggccagacatatggccctgaaacagatggtcagcctggcttcctgccatcctcggcctcaacctcgggcagtgtgacctatgaagggcactacagccacacagggcaggatggtcaagctgccttttccaaagatttttacggacccaactcccaaggttcacatgtggccagcggatttccagctgagcaggctgggggcctgaaaagtgaggtcgggccactgctgcagggcacaaacagccaatgggagagcccccatggattctcgggccaaagcaagcctgatctcacagcaggtcccatgtggcctccaccccacaaccagccctatgagctgtacgaccccgaggaaccaacctcagacaggacacctccttccttcgggggtcggcttaacaacagcaaacagggttttatcggtgctgggcggagggccaaggaggaccaggcgttgctatctgtgcggcctctgcaggctcatgagctgaacgactttcacggtgtggcccccctccacttgccgcatatctgtagcatctgtgacaagaaggtgtttgatttgaaggactgggagctgcatgtgaaagggaagctgcacgctcagaaatgcctggtcttctctgaaaatgctggcatccggtgtatacttggttcggcagagggaacattgtgtgcttctcccaacagcacagctgtttataaccctgctgggaatgaagattatgcctcaaatcttggaacatcatacgtgcccattccagcaaggtcattcactcagtcaagccccacatttcctttggcttctgtggggacaacttttgcacagcggaaaggggctggccgtgtggtgcacatctgcaatctccctgaaggaagctgcactgagaatgacgtcattaacctggggctgccctttggaaaggtcactaattacatcctcatgaaatcgactaatcaggcctttttagagatggcttacacagaagctgcacaggccatggtccagtattatcaagaaaaatctgctgtgatcaatggtgagaagttgctcattcggatgtccaagagatacaaggaattgcagctcaagaaacccgggaaggccgtggctgccatcatccaggacatccattcccagagggagagggacatgttccgggaagcagacagatatggcccagaaaggccgcggtctcgtagtccggtgagccggtcactctccccgaggtcccacactcccagcttcacctcctgcagctcttcccacagccctccgggcccctcccgggctgactggggcaatggccgggactcctgggagcactctccctatgccaggagggaggaagagcgagacccggctccctggagggacaacggagatgacaagagggacaggatggacccctgggcacatgatcgcaaacaccacccccggcaactggacaaggctgagttggacgagcgaccagaaggagggaggccccaccgggagaagtacccgagatctgggtctcccaacctgccccactctgtgtccagctacaaaagccgtgaagacggctactaccggaaagagcccaaagccaagtgggacaagtatctgaagcagcagcaggatgcccccgggaggtccaggaggaaagacgaggccaggctgcgggaaagcagacacccccatccggatgactcaggcaaggaagatgggctggggccaaaggtcactagggcccctgagggcgccaaggccaagcagaatgagaaaaataaaaccaagagaactgatagagaccaagaaggagctgatgatagaaaagaaaacacaatggcagagaatgaggctggaaaagaggaacaggagggcatggaagaaagccctcaatcagtgggcagacaggagaaagaagcagagttctctgatccggaaaacacaaggacaaagaaggaacaagattgggagagtgaaagtgaggcagagggggagagctggtatcccactaacatggaggagctggtgacagtggacgaggttggggaagaagaagattttatcgtggaaccagacatcccagagctggaagaaattgtgcccattgaccagaaagacaaaatttgcccagaaacatgtctgtgtgtgacaaccaccttagacttagacctggcccaggatttccccaaggaaggagtcaaggccgtagggaatggggctgcagaaatcagcctcaagtcacccagagaactgccctctgcttccacaagctgtcccagtgacatggacgtggaaatgcctggcctaaatctggatgctgagcggaagccagctgaaagtgagacaggcctctccctggaggattcagattgctacgagaaggaggcaaagggagtggagagctcagatgttcatccagcccctacagtccagcaaatgtcttcccctaagccagcagaggagagggcccggcagccaagcccatttgtggatgattgcaagaccagggggacccccgaagatggggcttgtgaaggcagccccctggaggagaaagccagcccccccatcgaaactgacctccaaaaccaagcctgccaagaagtgttgaccccggaaaactccaggtacgtggaaatgaaatctctggaggtgaggtcaccagagtacactgaagtggaactgaaacagcccctttctttgccctcttgggaaccagaggatgtgttcagtgaacttagcattcctctaggggtggagttcgtggttcccaggactggcttttattgcaagctgtgtgggctgttctacacgagcgaggagacagcaaagatgagccactgccgcagcgctgtccactacaggaacttacagaaatatttgtcccagctggccgaggagggcctcaaggagaccgagggggcagatagcccgaggccagaggacagcggaatcgtgccacgcttcgaaaggaaaaagctctgaaataaaagatccttattttcattggatctgtgtgttggttttttgtgtgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 42 - DNA, 4387 bp, synthetic construct
  source 1..4387: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcagccccaacatggtgctggcagcagccatgagccaggacgcggaccccagcggtccggagcagccggacagagttgcctgcagtgtgcctggtgcccgggcgtccccggcaccctccggcccgcgagggatgcagcagccgccgccgccgccccagccaccgcccccgccccaagccggcctaccccagatcatccaaaatgccgccaagctcctggacaagaacccattctcggtcagtaacccgaaccctctgcttccttcacctgccagtctccagctggctcaactgcaggcccagctcaccctccaccggctgaagctggcacagacagctgtcaccaacaacactgcagccgccacagtcctgaaccaagtcctctccaaagtggccatgtcccagcctctcttcaatcaactgaggcatccgtctgtgatcactggcccccacggccatgctggggttccccaacatgctgcagccatacccagtacccggtttccctctaatgcaattgccttttcaccccccagccagacacgaggccccggaccctccatgaaccttcccaaccagccacccagtgccatggtgatgcatcctttcactggggtaatgcctcagacccctggccagccagcagtcatcttgggcattggcaagactgggcctgctccagctacagcaggattctatgagtatggcaaagccagctctggccagacatatggccctgaaacagatggtcagcctggcttcctgccatcctcggcctcaacctcgggcagtgtgacctatgaagggcactacagccacacagggcaggatggtcaagctgccttttccaaagatttttacggacccaactcccaaggttcacatgtggccagcggatttccagctgagcaggctgggggcctgaaaagtgaggtcgggccactgctgcagggcacaaacagccaatgggagagcccccatggattctcgggccaaagcaagcctgatctcacagcaggtcccatgtggcctccaccccacaaccagccctatgagctgtacgaccccgaggaaccaacctcagacaggacacctccttccttcgggggtcggcttaacaacagcaaacagggttttatcggtgctgggcggagggccaaggaggaccaggcgttgctatctgtgcggcctctgcaggctcatgagctgaacgactttcacggtgtggcccccctccacttgccgcatatctgtagcatctgtgacaagaaggtgtttgatttgaaggactgggagctgcatgtgaaagggaagctgcacgctcagaaatgcctggtcttctctgaaaatgctggcatccggtgtatacttggttcggcagagggaacattgtgtgcttctcccaacagcacagctgtttataaccctgctgggaatgaagattatgcctcaaatcttggaacatcatacgtgcccattccagcaaggtcattcactcagtcaagccccacatttcctttggcttctgtggggacaacttttgcacagcggaaaggggctggccgtgtggtgcacatctgcaatctccctgaaggaagctgcactgagaatgacgtcattaacctggggctgccctttggaaaggtcactaattacatcctcatgaaatcgactaatcaggcctttttagagatggcttacacagaagctgcacaggccatggtccagtattatcaagaaaaatctgctgtgatcaatggtgagaagttgctcattcggatgtccaagagatacaaggaattgcagctcaagaaacccgggaaggccgtggctgccatcatccaggacatccattcccagagggagagggacatgttccgggaagcagacagatatggcccagaaaggccgcggtctcgtagtccggtgagccggtcactctccccgaggtcccacactcccagcttcacctcctgcagctcttcccacagccctccgggcccctcccgggctgactggggcaatggccgggactcctgggagcactctccctatgccaggagggaggaagagcgagacccggctccctggagggacaacggagatgacaagagggacaggatggacccctgggcacatgatcgcaaacaccacccccggcaactggacaaggctgagttggacgagcgaccagaaggagggaggccccaccgggagaagtacccgagatctgggtctcccaacctgccccactctgtgtccagctacaaaagccgtgaagacggctactaccggaaagagcccaaagccaagtgggacaagtatctgaagcagcagcaggatgcccccgggaggtccaggaggaaagacgaggccaggctgcgggaaagcagacacccccatccggatgactcaggcaaggaagatgggctggggccaaaggtcactagggcccctgagggcgccaaggccaagcagaatgagaaaaataaaaccaagagaactgatagagaccaagaaggagctgatgatagaaaagaaaacacaatggcagagaatgaggctggaaaagaggaacaggagggcatggaagaaagccctcaatcagtgggcagacaggagaaagaagcagagttctctgatccggaaaacacaaggacaaagaaggaacaagattgggagagtgaaagtgaggcagagggggagagctggtatcccactaacatggaggagctggtgacagtggacgaggttggggaagaagaagattttatcgtggaaccagacatcccagagctggaagaaattgtgcccattgaccagaaagacaaaatttgcccagaaacatgtctgtgtgtgacaaccaccttagacttagacctggcccaggatttccccaaggaaggagtcaaggccgtagggaatggggctgcagaaatcagcctcaagtcacccagagaactgccctctgcttccacaagctgtcccagtgacatggacgtggaaatgcctggcctaaatctggatgctgagcggaagccagctgaaagtgagacaggcctctccctggaggattcagattgctacgagaaggaggcaaagggagtggagagctcagatgttcatccagcccctacagtccagcaaatgtcttcccctaagccagcagaggagagggcccggcagccaagcccatttgtggatgattgcaagaccagggggacccccgaagatggggcttgtgaaggcagccccctggaggagaaagccagcccccccatcgaaactgacctccaaaaccaagcctgccaagaagtgttgaccccggaaaactccaggtacgtggaaatgaaatctctggaggtgaggtcaccagagtacactgaagtggaactgaaacagcccctttctttgccctcttgggaaccagaggatgtgttcagtgaacttagcattcctctaggggtggagttcgtggttcccaggactggcttttattgcaagctgtgtgggctgttctacacgagcgaggagacagcaaagatgagccactgccgcagcgctgtccactacaggaacttacagaaatatttgtcccagctggccgaggagggcctcaaggagaccgagggggcagatagcccgaggccagaggacagcggaatcgtgccacgcttcgaaaggaaaaagctctgaaataaaagatccttattttcattggatctgtgtgttggttttttgtgtgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 43 - DNA, 4386 bp, synthetic construct
  source 1..4386: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctccaacccagcatggtgctggcagcagccatgagccaggacgcggaccccagcggtccggagcagccggacagagttgcctgcagtgtgcctggtgcccgggcgtccccggcaccctccggcccgcgagggatgcagcagccgccgccgccgccccagccaccgcccccgccccaagccggcctaccccagatcatccaaaatgccgccaagctcctggacaagaacccattctcggtcagtaacccgaaccctctgcttccttcacctgccagtctccagctggctcaactgcaggcccagctcaccctccaccggctgaagctggcacagacagctgtcaccaacaacactgcagccgccacagtcctgaaccaagtcctctccaaagtggccatgtcccagcctctcttcaatcaactgaggcatccgtctgtgatcactggcccccacggccatgctggggttccccaacatgctgcagccatacccagtacccggtttccctctaatgcaattgccttttcaccccccagccagacacgaggccccggaccctccatgaaccttcccaaccagccacccagtgccatggtgatgcatcctttcactggggtaatgcctcagacccctggccagccagcagtcatcttgggcattggcaagactgggcctgctccagctacagcaggattctatgagtatggcaaagccagctctggccagacatatggccctgaaacagatggtcagcctggcttcctgccatcctcggcctcaacctcgggcagtgtgacctatgaagggcactacagccacacagggcaggatggtcaagctgccttttccaaagatttttacggacccaactcccaaggttcacatgtggccagcggatttccagctgagcaggctgggggcctgaaaagtgaggtcgggccactgctgcagggcacaaacagccaatgggagagcccccatggattctcgggccaaagcaagcctgatctcacagcaggtcccatgtggcctccaccccacaaccagccctatgagctgtacgaccccgaggaaccaacctcagacaggacacctccttccttcgggggtcggcttaacaacagcaaacagggttttatcggtgctgggcggagggccaaggaggaccaggcgttgctatctgtgcggcctctgcaggctcatgagctgaacgactttcacggtgtggcccccctccacttgccgcatatctgtagcatctgtgacaagaaggtgtttgatttgaaggactgggagctgcatgtgaaagggaagctgcacgctcagaaatgcctggtcttctctgaaaatgctggcatccggtgtatacttggttcggcagagggaacattgtgtgcttctcccaacagcacagctgtttataaccctgctgggaatgaagattatgcctcaaatcttggaacatcatacgtgcccattccagcaaggtcattcactcagtcaagccccacatttcctttggcttctgtggggacaacttttgcacagcggaaaggggctggccgtgtggtgcacatctgcaatctccctgaaggaagctgcactgagaatgacgtcattaacctggggctgccctttggaaaggtcactaattacatcctcatgaaatcgactaatcaggcctttttagagatggcttacacagaagctgcacaggccatggtccagtattatcaagaaaaatctgctgtgatcaatggtgagaagttgctcattcggatgtccaagagatacaaggaattgcagctcaagaaacccgggaaggccgtggctgccatcatccaggacatccattcccagagggagagggacatgttccgggaagcagacagatatggcccagaaaggccgcggtctcgtagtccggtgagccggtcactctccccgaggtcccacactcccagcttcacctcctgcagctcttcccacagccctccgggcccctcccgggctgactggggcaatggccgggactcctgggagcactctccctatgccaggagggaggaagagcgagacccggctccctggagggacaacggagatgacaagagggacaggatggacccctgggcacatgatcgcaaacaccacccccggcaactggacaaggctgagttggacgagcgaccagaaggagggaggccccaccgggagaagtacccgagatctgggtctcccaacctgccccactctgtgtccagctacaaaagccgtgaagacggctactaccggaaagagcccaaagccaagtgggacaagtatctgaagcagcagcaggatgcccccgggaggtccaggaggaaagacgaggccaggctgcgggaaagcagacacccccatccggatgactcaggcaaggaagatgggctggggccaaaggtcactagggcccctgagggcgccaaggccaagcagaatgagaaaaataaaaccaagagaactgatagagaccaagaaggagctgatgatagaaaagaaaacacaatggcagagaatgaggctggaaaagaggaacaggagggcatggaagaaagccctcaatcagtgggcagacaggagaaagaagcagagttctctgatccggaaaacacaaggacaaagaaggaacaagattgggagagtgaaagtgaggcagagggggagagctggtatcccactaacatggaggagctggtgacagtggacgaggttggggaagaagaagattttatcgtggaaccagacatcccagagctggaagaaattgtgcccattgaccagaaagacaaaatttgcccagaaacatgtctgtgtgtgacaaccaccttagacttagacctggcccaggatttccccaaggaaggagtcaaggccgtagggaatggggctgcagaaatcagcctcaagtcacccagagaactgccctctgcttccacaagctgtcccagtgacatggacgtggaaatgcctggcctaaatctggatgctgagcggaagccagctgaaagtgagacaggcctctccctggaggattcagattgctacgagaaggaggcaaagggagtggagagctcagatgttcatccagcccctacagtccagcaaatgtcttcccctaagccagcagaggagagggcccggcagccaagcccatttgtggatgattgcaagaccagggggacccccgaagatggggcttgtgaaggcagccccctggaggagaaagccagcccccccatcgaaactgacctccaaaaccaagcctgccaagaagtgttgaccccggaaaactccaggtacgtggaaatgaaatctctggaggtgaggtcaccagagtacactgaagtggaactgaaacagcccctttctttgccctcttgggaaccagaggatgtgttcagtgaacttagcattcctctaggggtggagttcgtggttcccaggactggcttttattgcaagctgtgtgggctgttctacacgagcgaggagacagcaaagatgagccactgccgcagcgctgtccactacaggaacttacagaaatatttgtcccagctggccgaggagggcctcaaggagaccgagggggcagatagcccgaggccagaggacagcggaatcgtgccacgcttcgaaaggaaaaagctctgaaataaaagatccttattttcattggatctgtgtgttggttttttgtgtgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 44 - DNA, 4173 bp, synthetic construct
  source 1..4173: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgatcactgggaccagaggaggggctggaggatactacacgcaggggtgggctgggctgggctgggctgggccaggaatgcagcggggcagggctatttaagtcaagggccggctggcaaccccagcaagctgtcctgtgagagcgccaccatggtgctggcagcagccatgagccaggacgcggaccccagcggtccggagcagccggacagagttgcctgcagtgtgcctggtgcccgggcgtccccggcaccctccggcccgcgagggatgcagcagccgccgccgccgccccagccaccgcccccgccccaagccggcctaccccagatcatccaaaatgccgccaagctcctggacaagaacccattctcggtcagtaacccgaaccctctgcttccttcacctgccagtctccagctggctcaactgcaggcccagctcaccctccaccggctgaagctggcacagacagctgtcaccaacaacactgcagccgccacagtcctgaaccaagtcctctccaaagtggccatgtcccagcctctcttcaatcaactgaggcatccgtctgtgatcactggcccccacggccatgctggggttccccaacatgctgcagccatacccagtacccggtttccctctaatgcaattgccttttcaccccccagccagacacgaggccccggaccctccatgaaccttcccaaccagccacccagtgccatggtgatgcatcctttcactggggtaatgcctcagacccctggccagccagcagtcatcttgggcattggcaagactgggcctgctccagctacagcaggattctatgagtatggcaaagccagctctggccagacatatggccctgaaacagatggtcagcctggcttcctgccatcctcggcctcaacctcgggcagtgtgacctatgaagggcactacagccacacagggcaggatggtcaagctgccttttccaaagatttttacggacccaactcccaaggttcacatgtggccagcggatttccagctgagcaggctgggggcctgaaaagtgaggtcgggccactgctgcagggcacaaacagccaatgggagagcccccatggattctcgggccaaagcaagcctgatctcacagcaggtcccatgtggcctccaccccacaaccagccctatgagctgtacgaccccgaggaaccaacctcagacaggacacctccttccttcgggggtcggcttaacaacagcaaacagggttttatcggtgctgggcggagggccaaggaggaccaggcgttgctatctgtgcggcctctgcaggctcatgagctgaacgactttcacggtgtggcccccctccacttgccgcatatctgtagcatctgtgacaagaaggtgtttgatttgaaggactgggagctgcatgtgaaagggaagctgcacgctcagaaatgcctggtcttctctgaaaatgctggcatccggtgtatacttggttcggcagagggaacattgtgtgcttctcccaacagcacagctgtttataaccctgctgggaatgaagattatgcctcaaatcttggaacatcatacgtgcccattccagcaaggtcattcactcagtcaagccccacatttcctttggcttctgtggggacaacttttgcacagcggaaaggggctggccgtgtggtgcacatctgcaatctccctgaaggaagctgcactgagaatgacgtcattaacctggggctgccctttggaaaggtcactaattacatcctcatgaaatcgactaatcaggcctttttagagatggcttacacagaagctgcacaggccatggtccagtattatcaagaaaaatctgctgtgatcaatggtgagaagttgctcattcggatgtccaagagatacaaggaattgcagctcaagaaacccgggaaggccgtggctgccatcatccaggacatccattcccagagggagagggacatgttccgggaagcagacagatatggcccagaaaggccgcggtctcgtagtccggtgagccggtcactctccccgaggtcccacactcccagcttcacctcctgcagctcttcccacagccctccgggcccctcccgggctgactggggcaatggccgggactcctgggagcactctccctatgccaggagggaggaagagcgagacccggctccctggagggacaacggagatgacaagagggacaggatggacccctgggcacatgatcgcaaacaccacccccggcaactggacaaggctgagttggacgagcgaccagaaggagggaggccccaccgggagaagtacccgagatctgggtctcccaacctgccccactctgtgtccagctacaaaagccgtgaagacggctactaccggaaagagcccaaagccaagtgggacaagtatctgaagcagcagcaggatgcccccgggaggtccaggaggaaagacgaggccaggctgcgggaaagcagacacccccatccggatgactcaggcaaggaagatgggctggggccaaaggtcactagggcccctgagggcgccaaggccaagcagaatgagaaaaataaaaccaagagaactgatagagaccaagaaggagctgatgatagaaaagaaaacacaatggcagagaatgaggctggaaaagaggaacaggagggcatggaagaaagccctcaatcagtgggcagacaggagaaagaagcagagttctctgatccggaaaacacaaggacaaagaaggaacaagattgggagagtgaaagtgaggcagagggggagagctggtatcccactaacatggaggagctggtgacagtggacgaggttggggaagaagaagattttatcgtggaaccagacatcccagagctggaagaaattgtgcccattgaccagaaagacaaaatttgcccagaaacatgtctgtgtgtgacaaccaccttagacttagacctggcccaggatttccccaaggaaggagtcaaggccgtagggaatggggctgcagaaatcagcctcaagtcacccagagaactgccctctgcttccacaagctgtcccagtgacatggacgtggaaatgcctggcctaaatctggatgctgagcggaagccagctgaaagtgagacaggcctctccctggaggattcagattgctacgagaaggaggcaaagggagtggagagctcagatgttcatccagcccctacagtccagcaaatgtcttcccctaagccagcagaggagagggcccggcagccaagcccatttgtggatgattgcaagaccagggggacccccgaagatggggcttgtgaaggcagccccctggaggagaaagccagcccccccatcgaaactgacctccaaaaccaagcctgccaagaagtgttgaccccggaaaactccaggtacgtggaaatgaaatctctggaggtgaggtcaccagagtacactgaagtggaactgaaacagcccctttctttgccctcttgggaaccagaggatgtgttcagtgaacttagcattcctctaggggtggagttcgtggttcccaggactggcttttattgcaagctgtgtgggctgttctacacgagcgaggagacagcaaagatgagccactgccgcagcgctgtccactacaggaacttacagaaatatttgtcccagctggccgaggagggcctcaaggagaccgagggggcagatagcccgaggccagaggacagcggaatcgtgccacgcttcgaaaggaaaaagctctgaaataaaagatccttattttcattggatctgtgtgttggttttttgtgtgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 45 - DNA, 4173 bp, synthetic construct
  source 1..4173: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgatcactgggaccagaggaggggctggaggatactacacgcaggggtgggctgggctgggctgggctgggccaggaatgcagcggggcagggctatttaagtcaagggccggctggcaaccccagcaagctgtcctgtgagagccccaacatggtgctggcagcagccatgagccaggacgcggaccccagcggtccggagcagccggacagagttgcctgcagtgtgcctggtgcccgggcgtccccggcaccctccggcccgcgagggatgcagcagccgccgccgccgccccagccaccgcccccgccccaagccggcctaccccagatcatccaaaatgccgccaagctcctggacaagaacccattctcggtcagtaacccgaaccctctgcttccttcacctgccagtctccagctggctcaactgcaggcccagctcaccctccaccggctgaagctggcacagacagctgtcaccaacaacactgcagccgccacagtcctgaaccaagtcctctccaaagtggccatgtcccagcctctcttcaatcaactgaggcatccgtctgtgatcactggcccccacggccatgctggggttccccaacatgctgcagccatacccagtacccggtttccctctaatgcaattgccttttcaccccccagccagacacgaggccccggaccctccatgaaccttcccaaccagccacccagtgccatggtgatgcatcctttcactggggtaatgcctcagacccctggccagccagcagtcatcttgggcattggcaagactgggcctgctccagctacagcaggattctatgagtatggcaaagccagctctggccagacatatggccctgaaacagatggtcagcctggcttcctgccatcctcggcctcaacctcgggcagtgtgacctatgaagggcactacagccacacagggcaggatggtcaagctgccttttccaaagatttttacggacccaactcccaaggttcacatgtggccagcggatttccagctgagcaggctgggggcctgaaaagtgaggtcgggccactgctgcagggcacaaacagccaatgggagagcccccatggattctcgggccaaagcaagcctgatctcacagcaggtcccatgtggcctccaccccacaaccagccctatgagctgtacgaccccgaggaaccaacctcagacaggacacctccttccttcgggggtcggcttaacaacagcaaacagggttttatcggtgctgggcggagggccaaggaggaccaggcgttgctatctgtgcggcctctgcaggctcatgagctgaacgactttcacggtgtggcccccctccacttgccgcatatctgtagcatctgtgacaagaaggtgtttgatttgaaggactgggagctgcatgtgaaagggaagctgcacgctcagaaatgcctggtcttctctgaaaatgctggcatccggtgtatacttggttcggcagagggaacattgtgtgcttctcccaacagcacagctgtttataaccctgctgggaatgaagattatgcctcaaatcttggaacatcatacgtgcccattccagcaaggtcattcactcagtcaagccccacatttcctttggcttctgtggggacaacttttgcacagcggaaaggggctggccgtgtggtgcacatctgcaatctccctgaaggaagctgcactgagaatgacgtcattaacctggggctgccctttggaaaggtcactaattacatcctcatgaaatcgactaatcaggcctttttagagatggcttacacagaagctgcacaggccatggtccagtattatcaagaaaaatctgctgtgatcaatggtgagaagttgctcattcggatgtccaagagatacaaggaattgcagctcaagaaacccgggaaggccgtggctgccatcatccaggacatccattcccagagggagagggacatgttccgggaagcagacagatatggcccagaaaggccgcggtctcgtagtccggtgagccggtcactctccccgaggtcccacactcccagcttcacctcctgcagctcttcccacagccctccgggcccctcccgggctgactggggcaatggccgggactcctgggagcactctccctatgccaggagggaggaagagcgagacccggctccctggagggacaacggagatgacaagagggacaggatggacccctgggcacatgatcgcaaacaccacccccggcaactggacaaggctgagttggacgagcgaccagaaggagggaggccccaccgggagaagtacccgagatctgggtctcccaacctgccccactctgtgtccagctacaaaagccgtgaagacggctactaccggaaagagcccaaagccaagtgggacaagtatctgaagcagcagcaggatgcccccgggaggtccaggaggaaagacgaggccaggctgcgggaaagcagacacccccatccggatgactcaggcaaggaagatgggctggggccaaaggtcactagggcccctgagggcgccaaggccaagcagaatgagaaaaataaaaccaagagaactgatagagaccaagaaggagctgatgatagaaaagaaaacacaatggcagagaatgaggctggaaaagaggaacaggagggcatggaagaaagccctcaatcagtgggcagacaggagaaagaagcagagttctctgatccggaaaacacaaggacaaagaaggaacaagattgggagagtgaaagtgaggcagagggggagagctggtatcccactaacatggaggagctggtgacagtggacgaggttggggaagaagaagattttatcgtggaaccagacatcccagagctggaagaaattgtgcccattgaccagaaagacaaaatttgcccagaaacatgtctgtgtgtgacaaccaccttagacttagacctggcccaggatttccccaaggaaggagtcaaggccgtagggaatggggctgcagaaatcagcctcaagtcacccagagaactgccctctgcttccacaagctgtcccagtgacatggacgtggaaatgcctggcctaaatctggatgctgagcggaagccagctgaaagtgagacaggcctctccctggaggattcagattgctacgagaaggaggcaaagggagtggagagctcagatgttcatccagcccctacagtccagcaaatgtcttcccctaagccagcagaggagagggcccggcagccaagcccatttgtggatgattgcaagaccagggggacccccgaagatggggcttgtgaaggcagccccctggaggagaaagccagcccccccatcgaaactgacctccaaaaccaagcctgccaagaagtgttgaccccggaaaactccaggtacgtggaaatgaaatctctggaggtgaggtcaccagagtacactgaagtggaactgaaacagcccctttctttgccctcttgggaaccagaggatgtgttcagtgaacttagcattcctctaggggtggagttcgtggttcccaggactggcttttattgcaagctgtgtgggctgttctacacgagcgaggagacagcaaagatgagccactgccgcagcgctgtccactacaggaacttacagaaatatttgtcccagctggccgaggagggcctcaaggagaccgagggggcagatagcccgaggccagaggacagcggaatcgtgccacgcttcgaaaggaaaaagctctgaaataaaagatccttattttcattggatctgtgtgttggttttttgtgtgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 46 - DNA, 4173 bp, synthetic construct
  source 1..4173: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgatcactgggaccagaggaggggctggaggatactacacgcaggggtgggctgggctgggctgggctgggccaggaatgcagcggggcagggctatttaagtcaagggccggctggcaaccccagcaagctgtcctgtgagcaacccagcatggtgctggcagcagccatgagccaggacgcggaccccagcggtccggagcagccggacagagttgcctgcagtgtgcctggtgcccgggcgtccccggcaccctccggcccgcgagggatgcagcagccgccgccgccgccccagccaccgcccccgccccaagccggcctaccccagatcatccaaaatgccgccaagctcctggacaagaacccattctcggtcagtaacccgaaccctctgcttccttcacctgccagtctccagctggctcaactgcaggcccagctcaccctccaccggctgaagctggcacagacagctgtcaccaacaacactgcagccgccacagtcctgaaccaagtcctctccaaagtggccatgtcccagcctctcttcaatcaactgaggcatccgtctgtgatcactggcccccacggccatgctggggttccccaacatgctgcagccatacccagtacccggtttccctctaatgcaattgccttttcaccccccagccagacacgaggccccggaccctccatgaaccttcccaaccagccacccagtgccatggtgatgcatcctttcactggggtaatgcctcagacccctggccagccagcagtcatcttgggcattggcaagactgggcctgctccagctacagcaggattctatgagtatggcaaagccagctctggccagacatatggccctgaaacagatggtcagcctggcttcctgccatcctcggcctcaacctcgggcagtgtgacctatgaagggcactacagccacacagggcaggatggtcaagctgccttttccaaagatttttacggacccaactcccaaggttcacatgtggccagcggatttccagctgagcaggctgggggcctgaaaagtgaggtcgggccactgctgcagggcacaaacagccaatgggagagcccccatggattctcgggccaaagcaagcctgatctcacagcaggtcccatgtggcctccaccccacaaccagccctatgagctgtacgaccccgaggaaccaacctcagacaggacacctccttccttcgggggtcggcttaacaacagcaaacagggttttatcggtgctgggcggagggccaaggaggaccaggcgttgctatctgtgcggcctctgcaggctcatgagctgaacgactttcacggtgtggcccccctccacttgccgcatatctgtagcatctgtgacaagaaggtgtttgatttgaaggactgggagctgcatgtgaaagggaagctgcacgctcagaaatgcctggtcttctctgaaaatgctggcatccggtgtatacttggttcggcagagggaacattgtgtgcttctcccaacagcacagctgtttataaccctgctgggaatgaagattatgcctcaaatcttggaacatcatacgtgcccattccagcaaggtcattcactcagtcaagccccacatttcctttggcttctgtggggacaacttttgcacagcggaaaggggctggccgtgtggtgcacatctgcaatctccctgaaggaagctgcactgagaatgacgtcattaacctggggctgccctttggaaaggtcactaattacatcctcatgaaatcgactaatcaggcctttttagagatggcttacacagaagctgcacaggccatggtccagtattatcaagaaaaatctgctgtgatcaatggtgagaagttgctcattcggatgtccaagagatacaaggaattgcagctcaagaaacccgggaaggccgtggctgccatcatccaggacatccattcccagagggagagggacatgttccgggaagcagacagatatggcccagaaaggccgcggtctcgtagtccggtgagccggtcactctccccgaggtcccacactcccagcttcacctcctgcagctcttcccacagccctccgggcccctcccgggctgactggggcaatggccgggactcctgggagcactctccctatgccaggagggaggaagagcgagacccggctccctggagggacaacggagatgacaagagggacaggatggacccctgggcacatgatcgcaaacaccacccccggcaactggacaaggctgagttggacgagcgaccagaaggagggaggccccaccgggagaagtacccgagatctgggtctcccaacctgccccactctgtgtccagctacaaaagccgtgaagacggctactaccggaaagagcccaaagccaagtgggacaagtatctgaagcagcagcaggatgcccccgggaggtccaggaggaaagacgaggccaggctgcgggaaagcagacacccccatccggatgactcaggcaaggaagatgggctggggccaaaggtcactagggcccctgagggcgccaaggccaagcagaatgagaaaaataaaaccaagagaactgatagagaccaagaaggagctgatgatagaaaagaaaacacaatggcagagaatgaggctggaaaagaggaacaggagggcatggaagaaagccctcaatcagtgggcagacaggagaaagaagcagagttctctgatccggaaaacacaaggacaaagaaggaacaagattgggagagtgaaagtgaggcagagggggagagctggtatcccactaacatggaggagctggtgacagtggacgaggttggggaagaagaagattttatcgtggaaccagacatcccagagctggaagaaattgtgcccattgaccagaaagacaaaatttgcccagaaacatgtctgtgtgtgacaaccaccttagacttagacctggcccaggatttccccaaggaaggagtcaaggccgtagggaatggggctgcagaaatcagcctcaagtcacccagagaactgccctctgcttccacaagctgtcccagtgacatggacgtggaaatgcctggcctaaatctggatgctgagcggaagccagctgaaagtgagacaggcctctccctggaggattcagattgctacgagaaggaggcaaagggagtggagagctcagatgttcatccagcccctacagtccagcaaatgtcttcccctaagccagcagaggagagggcccggcagccaagcccatttgtggatgattgcaagaccagggggacccccgaagatggggcttgtgaaggcagccccctggaggagaaagccagcccccccatcgaaactgacctccaaaaccaagcctgccaagaagtgttgaccccggaaaactccaggtacgtggaaatgaaatctctggaggtgaggtcaccagagtacactgaagtggaactgaaacagcccctttctttgccctcttgggaaccagaggatgtgttcagtgaacttagcattcctctaggggtggagttcgtggttcccaggactggcttttattgcaagctgtgtgggctgttctacacgagcgaggagacagcaaagatgagccactgccgcagcgctgtccactacaggaacttacagaaatatttgtcccagctggccgaggagggcctcaaggagaccgagggggcagatagcccgaggccagaggacagcggaatcgtgccacgcttcgaaaggaaaaagctctgaaataaaagatccttattttcattggatctgtgtgttggttttttgtgtgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 47 - skipped

(19) United States
(12) Patent Application Publication
(10) Pub. No.: US 2025/0205365 A1
(43) Pub. Date: Jun. 26, 2025

(54) METHODS AND COMPOSITIONS FOR TREATING RBM20 RELATED CARDIOMYOPATHY WITH A VIRAL VECTOR

(71) Applicants: University of Florida Research Foundation, Incorporated, Gainesville, FL (US); AavantiBio, Inc., Charlestown, MA (US)

(72) Inventors: Barry John Byrne, Gainesville, FL (US); Manuela Corti, Gainesville, FL (US); Widler Casy, Charlestown, MA (US)

(73) Assignees: University of Florida Research Foundation, Incorporated, Gainesville, FL (US); AavantiBio, Inc., Charlestown, MA (US)

(21) Appl. No.: 18/847,739

(22) PCT Filed: Mar. 18, 2023
(86) PCT No.: PCT/US2023/064671

§ 371 Date: Sep. 17, 2024

Related U.S. Application Data
(60) Provisional application No. 63/321,243, filed on Mar. 18, 2022.

(51) Int. Cl. A61K 48/00; A61K 9/00; A61K 38/17; A61P 9/00; C12N 15/86

(52) U.S. Cl. CPC A61K 48/0058; A61K 9/0019; A61K 38/1709; A61K 48/0075; A61K 48/0083; A61P 9/00; C12N 15/86; C12N 2750/14143; C12N 2800/22; C12N 2830/008; C12N 2830/50

(57) ABSTRACT

The present disclosure relates to compositions and methods for the treatment of cardiomyopathy. Several embodiments provided for herein relate to virally-mediated transfer of a gene to host cells to induce expression of an encoded polypeptide, protein or other product in order to ameliorate one or more symptoms of the cardiomyopathy in a subject. In several embodiments, the disclosed methods and compositions relate to recombinant adeno-associated virus particles encoding human RBM20 in order to treat cardiomyopathies, including dilated cardiomyopathy.

### PRIORITY

This application claims priority to U.S. Provisional Patent Application No. 63/321,243, filed Mar. 18, 2022, the entire contents of which is incorporated by reference herein.

### BACKGROUND

Cardiomyopathy represents a collection of diverse conditions of the heart muscle and is the second most common cause of heart disease in subjects and medical management of the secondary signs is the only therapeutic option. These diseases have many causes, symptoms, and treatments, and can affect people of all ages and races. When cardiomyopathy occurs, the normal muscle in the heart can thicken, stiffen, thin out, or fill with substances the body produces that do not belong in the heart muscle. As a result, the heart muscle's ability to pump blood is reduced, which can lead to irregular heartbeats, the backup of blood into the lungs or rest of the body, and heart failure. Cardiomyopathy can be acquired or inherited. The cause isn't always known but there is an increasing understanding of the genetic underpinnings of inherited forms of disease.

Gene transfer strategies have been shown to ameliorate heart disease.

### INCORPORATION BY REFERENCE OF MATERIAL IN SEQUENCE LISTING FILE

This application incorporates by reference the material in the Sequence Listing contained in the following XML file being submitted concurrently herewith: File name: U120270089WO00-SEQ-PRW.xml; created on Mar. 15, 2023 and is 82,768 bytes in size.

### SUMMARY

Cardiomyopathy is a class of disease of heart muscle that adversely impacts the heart's ability to circulate blood through the cardiovascular system. Various types of cardiomyopathies exist, including dilated cardiomyopathy, hypertrophic cardiomyopathy, and restrictive cardiomyopathy. Cardiomyopathy in human populations is a major medical burden and treatment needs are currently unmet, despite cardiomyopathies in human populations being particularly desirable to treat.

Dilated cardiomyopathy (DCM) is one of the most common types of human cardiomyopathy, occurring mostly in adults 20 to 60. DCM affects the heart's ventricles and atria, the lower and upper chambers of the heart, respectively. Most forms of DCM are acquired forms from a number of causes that include coronary heart disease, heart attack, high blood pressure, diabetes, thyroid disease, viral hepatitis and viral infections that inflame the heart muscle. Alcohol abuse and certain drugs, such as cocaine and amphetamines, as well as at least two drugs used to treat cancer (doxorubicin and daunorubicin), can also lead to DCM. In addition, there are a number of genetic forms of DCM, including, but not limited to the DCM associated with Duchenne and Becker muscular dystrophies. In certain forms of Becker muscular dystrophy, as well as in most cases of Duchenne muscular dystrophy, the cardiomyopathy can ultimately limit the patient's survival.

Hypertrophic cardiomyopathy (HCM) occurs when the walls of the heart muscle become abnormally thick. The increase in wall thickness may increase cardiac complications, as well as block or obstruct blood flowing in the heart.

Restrictive cardiomyopathy (RCM) is a condition leading to a stiffening of the chambers of the heart over time. While the heart's ability to contract remains largely unaffected, the cardiac muscle does not fully relax between beats of the heart. This restricts the ability of the ventricles to fill with blood and causes blood to back up in the circulatory system.

Heart function is critically dependent upon calcium-dependent signaling. During heart disease, malfunctioning of calcium channels within cardiac cells promotes calcium cycling abnormalities, further inhibiting heart function. Gene transfer strategies to reduce calcium cycling abnormalities are reported to ameliorate heart disease in small and large animal models, as well as in human clinical trials.

Disclosed herein are gene delivery approaches for treatment of human subjects with one or more types of cardiomyopathy or symptoms thereof.

Accordingly, some aspects of the present disclosure provide recombinant adeno-associated virus (rAAV) vectors for delivering transgenes into the heart of a subject. Such rAAV vectors may include, from 5′ to 3′, in order, a first adeno-associated virus (AAV) inverted terminal repeat (ITR) sequence, a promoter operably linked to one or more transgenes, and a second AAV inverted terminal repeat (ITR) sequence. In some embodiments, the rAAV vector includes, in addition to a promoter, a regulatory element which modifies expression, e.g., in a manner that provides physiologically relevant expression levels and/or restricts expression to a particular cell type or tissue. In some embodiments, the regulatory element comprises one or more of an enhancer, a 5′ untranslated region (UTR), and a 3′ UTR. In some embodiments, the UTR is a MHCK9 UTR, e.g., a 5′ MHCK9 UTR. In some embodiments, the rAAV vector also includes at least one polyadenylation signal (e.g., positioned 3′ of the one or more transgenes). In some embodiments, two transgenes arc operably linked to the same single promoter. In some embodiments, each transgene is operably linked to a separate promoter. In some embodiments in which multiple transgenes are provided, the rAAV vector also includes at least one polyadenylation signal (e.g., positioned 3′ of two transgenes expressed from a single promoter or 3′ of one or both transgenes expressed from different promoters). Aspects of the disclosure provide recombinant adeno-associated virus (rAAV) nucleic acid vectors for delivering two or more transgenes into the heart of a subject, wherein said vector comprises, from 5′ to 3′, a first adeno-associated virus (AAV) inverted terminal repeat (ITR) sequence, two or more transgenes and a promoter operably linked to the two or more transgenes, a polyadenylation signal, and a second AAV inverted terminal repeat (ITR) sequence.

In some embodiments, described herein is a nucleic acid comprising an expression construct comprising a human RBM20 coding sequence and an enhancer element, such as a CMV enhancer, operably linked to a promoter, wherein the expression construct is flanked on each side by an inverted terminal repeat sequence. In some embodiments, described herein is a nucleic acid comprising an expression construct comprising a human RBM20 coding sequence, an enhancer element operably linked to a promoter, and a Kozak sequence, wherein the Kozak sequence enhances transgene expression in the heart, wherein the expression construct is flanked on each side by an inverted terminal repeat sequence, wherein the Kozak sequence is non-native with respect to the human RBM20 coding sequence and/or non-native to the promoter. In some embodiments, described herein is a nucleic acid comprising an expression construct comprising a human RBM20 coding sequence, an enhancer element operably linked to a promoter, and an in silico designed consensus Kozak sequence, wherein the in silico designed consensus Kozak sequence enhances transgene expression in the heart, wherein the expression construct is flanked on each side by an inverted terminal repeat sequence, wherein the Kozak sequence is non-native with respect to the human RBM20 coding sequence and the promoter. In some embodiments, described herein is a nucleic acid comprising an expression construct comprising a human RBM20 coding sequence, an enhancer element operably linked to a promoter, and a Kozak sequence, wherein the Kozak sequence enhances transgene expression in the heart, wherein the expression construct is flanked on each side by an inverted terminal repeat sequence, wherein the Kozak sequence is native with respect to the human RBM20 coding sequence and/or native to the promoter. In several embodiments, the Kozak sequence is a synthetic sequence. In some embodiments, the human RBM20 coding sequence is codon-optimized for expression in human cells. In some embodiments, the promoter comprises a cardiac specific promoter. In some embodiments, the promoter is CBA (Chicken β-Actin), or a truncated chicken beta-actin (smCBA). In some embodiments, the nucleic acid is a recombinant adeno-associated virus (rAAV) vector. In some embodiments, the nucleic acid is a single-stranded or self-complementary rAAV nucleic acid vector. In some embodiments, the rAAV particle is an AAV9 particle. In some embodiments, the rAAV particle is an rh74 (or AAVrh74) particle. In some embodiments, the rAAV particle is an rh10 (or AAVrh10) particle. In some embodiments, a composition comprising a plurality of rAAV particles is provided. In some embodiments, the plurality of rAAV particles may further comprise a pharmaceutically acceptable carrier. In some embodiments, the rh74 particle comprises at least one capsid protein encoded by a polynucleotide having at least 85%, at least 90%, at least 95%, at least 96%, at least 97%, at least 98% or at least 99% sequence identity to the nucleotide sequence set forth as SEQ ID NO: 10, or a portion of SEQ ID NO: 10 (for example, SEQ ID NO: 10 encodes the rh74 VP1, VP2, and VP3 proteins-thus, in several embodiments, an rh74 particle according to embodiments disclosed herein comprises at least one capsid protein encoded by a polynucleotide having at least 85%, at least 90%, at least 95%, at least 96%, at least 97%, at least 98% or at least 99% sequence identity to a subpart of the nucleotide sequence of SEQ ID NO: 10). In some embodiments, the rh74 particle comprises an amino acid sequence having at least about 85%, at least 90%, at least 95%, at least 96%, at least 97%, at least 98% or at least 99% sequence identity to the amino acid sequence set forth as SEQ ID NO: 11, or a portion of SEQ ID NO: 11 (for example, SEQ ID NO: 11 is the amino acid sequence of rh74 VP1, VP2, and VP3 proteins-thus, in several embodiments, an rh74 particle according to embodiments disclosed herein comprises at least one capsid protein having at least 85%, at least 90%, at least 95%, at least 96%, at least 97%, at least 98% or at least 99% sequence identity to a subpart of the amino acid sequence of SEQ ID NO: 11). In some embodiments, the AAV9 particle comprises an amino acid sequence having at least about 85%, at least 90%, at least 95%, at least 96%, at least 97%, at least 98% or at least 99% sequence identity to the amino acid sequence set forth as SEQ ID NO: 12.

In some embodiments, the therapeutic transgene is encoded by a polynucleotide having at least 85%, at least 90%, at least 95%, at least 96%, at least 97%, at least 98% or at least 99% sequence identity to the nucleotide sequence set forth as SEQ ID NO: 5 (RBM20 cDNA). In some embodiments, one or more of the transgenes of the present disclosure are naturally-occurring sequences. In some embodiments, one or more transgenes are engineered to be species-specific. In some embodiments, one or more transgenes are codon-optimized for expression in a species of interest, e.g., human. For example, in several embodiments, the therapeutic transgene (e.g., the RBM20 transgene) is codon-optimized.

Further provided herein are rAAV particles containing any of the rAAV vectors disclosed herein, encapsidated in an AAV capsid protein. Other aspects of the present disclosure include compositions containing any of the nucleic acid vectors or the rAAV particles described herein. In several embodiments, such compositions may be administered to a subject for gene therapy for cardiomyopathy. In additional embodiments, such compositions may be administered to a subject for gene therapy for heart disease. In some embodiments, the heart disease causes heart failure in the subject.

The compositions of the present disclosure may be administered to the subject via different routes. In some embodiments, the composition is administered via intravenous injection into the subject. In some embodiments, the administration of the composition results in expression of the transgene (or, if multiple transgenes are used, expression of two or more transgenes) in the subject's heart. In various embodiments, the step of administering the composition results in improved cardiac function in the subject, such as improved cardiac function in the subject for more than 10 months. In some embodiments, administration results in improved cardiac function for more than 12 months, more than 14 months, more than 16 months, more than 17 months, more than 20 months, more than 22 months, or more than 24 months. In several embodiments, improved cardiac function is represented by an increase in left ventricular ejection fraction (LVEF). In several embodiments, the LVEF (as compared to a pre-therapy measurement) increases by at least about 1%, about 2%, about 3%, about 4%, about 5% or more (including any amount between those listed). In several embodiments, LVEF is measured by echocardiography. In some embodiments, administration results in improved cardiac physiology (e.g., structural features) for more than 12 months, more than 14 months, more than 16 months, more than 17 months, more than 20 months, more than 22 months, or more than 24 months. In several embodiments, the improved cardiac physiology is represented by a decrease in left ventricular wall thickness. In several embodiments, left ventricular wall thickness is reduced by at least about 1%, about 2%, about 3%, about 4%, about 5% or more (including any amount between those listed). In several embodiments, the left ventricular wall thickness is measured by cardiac magnetic resonance imaging (MRI) or transthoracic echocardiography (TTE).

In some embodiments, described herein are compositions comprising AAV vectors, virions, viral particles, and pharmaceutical formulations thereof, useful in methods for delivering genetic material encoding one or more beneficial or therapeutic product(s) to mammalian cells and tissues. Any of the rAAV vectors, rAAV particles, or compositions comprising the rAAV particles of the present disclosure may be used for gene therapy for treatment of one or more heart diseases, such as one or more types of cardiomyopathy. Any of the rAAV vectors, rAAV particles, or compositions comprising the rAAV particles of the present disclosure may be administered to a subject in need thereof, such as a human subject suffering from a heart disease such as a cardiomyopathy.

Additionally, provided herein are compositions, as well as therapeutic and/or diagnostic kits that include one or more of the disclosed AAV compositions, formulated with one or more additional ingredients, or prepared with one or more instructions for their use.

In some embodiments, described herein is a nucleic acid comprising an expression construct comprising a human RBM20 coding sequence, one or more silencing elements, and an enhancer element, such as a CMV enhancer, operably linked to a promoter, wherein the expression construct is flanked on each side by an inverted terminal repeat sequence. In some embodiments, the silencing elements comprise an shRNA expression cassette. In some embodiments, the silencing elements comprise an shRNA sequence. In some embodiments, the human RBM20 coding sequence is codon-optimized for expression in human cells. In some embodiments, the promoter comprises a cardiac specific promoter. In some embodiments, the promoter is TNNT2. In some embodiments, the promoter is CBA (Chicken β-Actin). In some embodiments, the promoter is CMV or mini-CMV. In some embodiments, the promoter is Desmin. In some embodiments, the promoter is a muscle creatine kinase (MCK) promoter. In some embodiments, the promoter is MHCK7. In some embodiments, the promoter is MHCK9. In some embodiments, the nucleic acid is a recombinant adeno-associated virus (rAAV) vector. In some embodiments, the nucleic acid is a single-stranded or self-complementary rAAV nucleic acid vector. In some embodiments, the expression construct is pTR-TNNT2-RBM20. In some embodiments, the expression construct is pTR2-MCHK9-RBM20.

In some embodiments, the rAAV particle is an AAV9 particle. In some embodiments, the rAAV particle is an rh74 particle. In some embodiments, the rAAV particle is an rh10 particle. In some embodiments, a composition comprising a plurality of rAAV particles is provided. In some embodiments, the plurality of rAAV particles may further comprise a pharmaceutically acceptable carrier. In some embodiments, the rh74 particle comprises at least one capsid protein encoded by a polynucleotide having at least 85%, at least 90%, at least 95%, at least 96%, at least 97%, at least 98% or at least 99% sequence identity to the nucleotide sequence set forth as SEQ ID NO: 10, or a portion of SEQ ID NO: 10. For example, SEQ ID NO: 10 encodes the rh74 VP1 protein, which also includes the VP2 and VP3 proteins-thus, in several embodiments, an rh74 particle according to embodiments disclosed herein comprises at least one capsid protein encoded by a polynucleotide having at least 85%, at least 90%, at least 95%, at least 96%, at least 97%, at least 98% or at least 99% sequence identity to a subpart of the nucleotide sequence of SEQ ID NO: 10. In some embodiments, the rh74 particle comprises an amino acid sequence having at least about 85%, at least 90%, at least 95%, at least 96%, at least 97%, at least 98% or at least 99% sequence identity to the amino acid sequence set forth as SEQ ID NO: 11, or a portion of SEQ ID NO: 11. For example, SEQ ID NO: 11 is the amino acid sequence of rh74 VP1 protein (including the VP2 and VP3 proteins)—thus, in several embodiments, an rh74 particle according to embodiments disclosed herein comprises at least one capsid protein having at least 85%, at least 90%, at least 95%, at least 96%, at least 97%, at least 98% or at least 99% sequence identity to a subpart of the amino acid sequence of SEQ ID NO: 11. In some embodiments, the AAV9 particle comprises an amino acid sequence having at least about 85%, at least 90%, at least 95%, at least 96%, at least 97%, at least 98% or at least 99% sequence identity to the amino acid sequence set forth as SEQ ID NO: 12.

In some embodiments, a method of treating dilated cardiomyopathy is described, the method comprising administering a therapeutically effective amount of rAAV comprising a nucleic acid expression construct comprising a human RBM20 coding sequence operably linked to a promoter and optionally and an enhancer element, wherein the expression construct is flanked on each side by an inverted terminal repeat sequence, and wherein said administration results in expression of a therapeutically effective amount of human RBM20, thereby treating the dilated cardiomyopathy. In some embodiments, the rAAV is administered via intravenous injection.

In some embodiments, a method of treating dilated cardiomyopathy is described, the method comprising administering a therapeutically effective amount of rAAV comprising a nucleic acid expression construct comprising a human RBM20 coding sequence, a silencing element, each element operably linked to a promoter and optionally comprising and an enhancer element, wherein the expression construct is flanked on each side by an inverted terminal repeat sequence, and wherein said administration results in expression of a therapeutically effective amount of human RBM20, thereby treating the dilated cardiomyopathy. In some embodiments of the disclosed methods, a therapeutically effective amount of rAAV comprising a nucleic acid expression construct is administered to a subject (e.g., a human) to treat dilated cardiomyopathy in the subject.

In some embodiments, a method of treating dilated cardiomyopathy is described, the method comprising administering a therapeutically effective amounts of (1) a silencing construct, e.g., an rAAV comprising a silencing construct, and (2) an rAAV comprising a nucleic acid expression construct comprising a human RBM20 coding sequence operably linked to a promoter and optionally an enhancer element, wherein the expression construct is flanked on each side by an inverted terminal repeat sequence, and wherein said administration results in expression of a therapeutically effective amount of human RBM20, thereby treating the dilated cardiomyopathy. In some embodiments, the rAAV is administered via intravenous injection.

In some embodiments, the rAAV, e.g., comprising a RBM20 coding sequence and/or the silencing construct are administered via intravenous injection. In some embodiments, between about 1×10¹³ and about 1×10¹⁴ rAAV vector genomes are administered. In some embodiments, at 20%, at least 30%, at least 40%, or at least 50% of cardiomyocyte cells are transduced when the rAAV vector genomes are administered. In some embodiments, at 20%, at least 30%, at least 40%, or at least 50% of cardiomyocyte cells are transduced when between about 1×10¹³ and about 1×10¹⁴ rAAV vector genomes are administered.

Also described herein is a method of increasing expression of human RBM20 in a target cell, comprising contacting a target cell with a plurality of rAAV particles comprising a nucleic acid expression construct comprising a functional human RBM20 coding sequence, a silencing element, and an enhancer element operably linked to a promoter, wherein the expression construct is flanked on each side by an inverted terminal repeat sequence, and wherein said contacting results in the target cell increasing expression of functional human RBM20 as compared to prior to the contacting, thereby increasing the expression of functional human RBM20.

Also described herein is a method of increasing expression of human RBM20 in a target cell, comprising contacting a target cell with a plurality of rAAV particles comprising a nucleic acid expression construct comprising a functional human RBM20 coding sequence operably linked to a promoter and optionally an enhancer element, wherein the expression construct is flanked on each side by an inverted terminal repeat sequence, and wherein said contacting results in the target cell increasing expression of functional human RBM20 as compared to prior to the contacting, thereby increasing the expression of functional human RBM20.

Also described herein is a method of increasing expression of human RBM20 in a target cell, comprising contacting a target cell with a plurality of silencing constructs and rAAV particles, wherein the rAAV particles comprise a nucleic acid expression construct comprising a functional human RBM20 coding sequence operably linked to a promoter and optionally an enhancer element, wherein the expression construct is flanked on each side by an inverted terminal repeat sequence, and wherein said contacting results in the target cell increasing expression of functional human RBM20 as compared to prior to the contacting, thereby increasing the expression of functional human RBM20.

In some embodiments, the contacting is in vivo. In some embodiments, the method is used for the treatment of dilated cardiomyopathy. In some embodiments, the nucleic acids, the rAAV particles, the compositions, or the methods of manufacture described herein can be used for the treatment of dilated cardiomyopathy. In some embodiments, the nucleic acids, the rAAV particles, the compositions, or the methods of manufacture described herein can be used for the treatment of idiopathic DCM. In some embodiments, the nucleic acids, the rAAV particles, the compositions, or the methods of manufacture described herein can be used for the treatment of DCM associated with Duchenne muscular dystrophy or Becker muscular dystrophy. In some embodiments, the nucleic acids, the rAAV particles, the compositions, or the methods of manufacture described herein can be used for the treatment of hypertrophic cardiomyopathy or restrictive cardiomyopathy.

Further provided herein are uses of any of the disclosed nucleic acids, rAAV particles, or compositions for the treatment of DCM, or in the manufacture of a medicament for the treatment of DCM.

### BRIEF DESCRIPTION OF THE DRAWINGS

FIG. 1 shows a non-limiting example of a gene construct map for an expression construct embodiment disclosed herein.

FIG. 2 shows a second non-limiting example of a gene construct map for an expression construct embodiment disclosed herein.

### DETAILED DESCRIPTION

Reference is made to particular features and/or non-limiting embodiments of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

A “subject” refers to mammal that is the object of treatment using a method or composition as provided for herein. “Mammal” includes, without limitation, mice, rats, rabbits, guinea pigs, dogs, cats, sheep, goats, cows, horses, primates, such as monkeys, chimpanzees, and apes, and humans. In some embodiments, the subject is human.

The terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy. To “treat” a disease as the term is used herein, means to reduce the frequency or severity of at least one sign or symptom of a disease or disorder experienced by a subject.

The term “effective amount,” as used herein, refers to an amount that is capable of treating or ameliorating a disease or condition or otherwise capable of producing an intended therapeutic effect, such as reducing the frequency or severity of at least one sign or symptom of a disease or disorder experienced by a subject.

A “nucleic acid” sequence refers to a deoxyribonucleic acid (DNA) or ribonucleic acid (RNA) sequence. This term encompasses naturally-occurring and non-naturally occurring nucleobases (bases). This term encompasses sequences that include any of the known base analogues of DNA and RNA such as, but not limited to 4-acetylcytosinc, 8-hydroxy-N6-methyladenosinc, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “polynucleotide,” refers to a polymeric form of nucleotides of any length, including DNA, RNA, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

For the purpose of describing the relative position of nucleotide sequences in a particular nucleic acid molecule throughout the instant application, such as when a particular nucleotide sequence is described as being situated “upstream,” “downstream,” “3′,” or “5” relative to another sequence, it is to be understood that it is the position of the sequences in the “sense” or “coding” strand of a DNA molecule that is being referred to as is conventional in the art.

The term “isolated” when referring to a nucleotide sequence, means that the indicated molecule is present in the substantial absence of other biological macromolecules of the same type. Thus, an “isolated nucleic acid molecule which encodes a particular polypeptide” refers to a nucleic acid molecule which is substantially free of other nucleic acid molecules that do not encode the subject polypeptide; however, the molecule may include some additional bases or moieties which do not materially affect the basic characteristics of the composition.

As used herein, the term “variant” refers to a molecule (e.g., a nucleic acid sequence or a protein sequence) having characteristics that deviate from what occurs in nature, e.g., a “variant” is at least about 80% identical, at least about 90% identical, at least about 95% identical, at least about 96% identical, at least about 97% identical, at least about 98% identical, at least about 99% identical, at least about 99.5% identical, or at least about 99.9% identical to the wild type counterpart. Variants of a nucleic acid or protein molecule may contain modifications to the sequence (e.g., having 1, 2, 3, 4, 5, 6, 7, 8, 9, 10, 10-15, or 15-20 base or amino acid substitutions, respectively) relative to the wild type sequence. These modifications include chemical modifications as well as truncations.

The term “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The “percent (%) identity” of two sequences, whether nucleic acid or amino acid sequences, is the number of exact matches between two aligned sequences divided by the length of the shorter sequences and multiplied by 100. This term refers to the extent to which two sequences (nucleotide or amino acid) have the same residue at the same positions in an alignment. For example, “an amino acid sequence is X % identical to SEQ ID NO: Y” refers to % identity of the amino acid sequence to SEQ ID NO: Y and is elaborated as X % of residues in the amino acid sequence are identical to the residues of sequence disclosed in SEQ ID NO: Y. Generally, computer programs are employed for such calculations. Sequence identity can be determined by aligning sequences using algorithms, such as BESTFIT, FASTA, and TFASTA in the Wisconsin Genetics Software Package Release 7.0, Genetics Computer Group, 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated the window of comparison between two sequences is defined by the entire length of the shorter of the two sequences.

The term “recombinant,” as applied to a polynucleotide means that the polynucleotide is the product of various combinations of cloning, restriction or ligation steps, and other procedures that result in a construct that is distinct from a polynucleotide found in nature and/or a combination of polynucleotides and viral proteins that is not found in nature. A recombinant virus is a viral particle comprising a recombinant polynucleotide. The terms respectively include replicates of the original polynucleotide construct and progeny of the original virus construct.

The term “gene,” refers to a polynucleotide containing at least one open reading frame that is capable of encoding a particular gene product. Any of the polynucleotide sequences described herein may be used to identify larger fragments or full-length coding sequences of the genes with which they are associated. Methods of isolating larger fragment sequences are known to those of skill in the art.

The term “transgene,” as used herein, refers to a nucleic acid sequence to be positioned within a viral vector and encoding a polypeptide, protein or other product of interest. In some embodiments, one rAAV vector may comprise a sequence encoding one or more transgenes (which can optionally be the same gene, or different genes). For example, one rAAV vector may comprise the coding sequence for 1, 2, 3, 4, 5, 6, 7, 8, 9, or 10 transgenes. The transgenes of the present disclosure relate to the improvement of one or more heart conditions, such as cardiomyopathies as provided for herein.

The terms “gene transfer” or “gene delivery” refer to methods or systems for inserting DNA, such as a transgene, into host cells, such as those of a subject afflicted with a cardiomyopathy. In several embodiments, gene transfer yields transient expression of non-integrated transferred DNA, extrachromosomal replication and expression of transferred replicons (e.g., episomes). In additional embodiments, gene transfer results in integration of transferred genetic material into the genomic DNA of host cells.

The terms “regulatory element” or “regulatory sequence”, or variations thereof, refer to a nucleotide sequence that participates in functional regulation of a polynucleotide, including replication, duplication, transcription, splicing, translation, or degradation of the polynucleotide. Regulatory elements can be enhancing or inhibitory in nature, depending on the embodiment. Non-limiting examples of regulatory elements include transcriptional regulatory sequences such as promoter sequences, polyadenylation signals, transcription termination sequences, upstream regulatory domains, origins of replication, internal ribosome entry sites (“IRES”), enhancers, and the like. These elements collectively provide for the replication, transcription and translation of a coding sequence in a recipient cell, though not all of these sequences need always be present. It shall be appreciated that the structural components of a rAAV vector as provided for herein may be listed in individual paragraphs solely for clarity and may be used together in combination. For example, any regulatory element or other component can be used in combination with any transgene (or transgenes) provided for herein.

A “promoter” is a polynucleotide that interacts with an RNA polymerase and initiates transcription of a coding region (e.g., a transgene) usually located downstream (in the 3′ direction) from the promoter.

The term “operably linked” refers to an arrangement of elements wherein the components are configured to perform a function. For example, regulatory sequences operably linked to a coding sequence result in the expression of the coding sequence. Depending on the embodiment, a regulatory sequence need not be contiguous with the coding sequence. Thus, for example, one or more untranslated, yet transcribed, sequences can be present between a promoter sequence and a coding sequence, with those two sequences still being considered “operably linked”.

The term “vector” means any molecular vehicle, such as a plasmid, phage, transposon, cosmid, chromosome, virus, viral particle, virion, etc. which can transfer gene sequences (e.g., a transgene) to or between cells of interest.

An “expression vector” is a vector comprising a region of nucleic acid (e.g., a transgene) which encodes a gene product (e.g., a polypeptide or protein) of interest. As disclosed herein, vectors are used for achieving expression, e.g., stable expression, of a protein in an intended target cell. An expression vector may also comprise control elements operatively linked to the transgene to facilitate expression of the encoded protein in the target cell. A combination of one or more regulatory elements and a gene or genes to which they are operably linked for expression may be referred to herein as an “expression cassette.”

The term “AAV” is an abbreviation for adeno-associated virus, and may be used to refer to the virus itself or derivatives thereof. The term covers all subtypes and both naturally occurring and recombinant forms, unless otherwise indicated. The abbreviation “rAAV” refers to recombinant adeno-associated virus, also referred to as a recombinant AAV vector (or “rAAV vector”), which refers to AAV comprising a polynucleotide sequence not of AAV origin (e.g., a transgene). The term “AAV” includes AAV serotype 1 (AAV1), AAV serotype 2 (AAV2), AAV serotype 3 (AAV3), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), serotype rh10 AAV, serotype rh74 AAV, or a pseudotyped rAAV (e.g., AAV2/9, referring an AAV vector with the genome of AAV2 (e.g., the ITRs of AAV2) and the capsid of AAV9). In several embodiments, the preferred serotype for delivery to human patients affected by a cardiomyopathy is one of AAV9, serotype rh74, serotype rh10, or AAV8. In several embodiments, an rh74 AAV is mutated to advantageously enhance delivery to cardiac tissue, for example by a tryptophan to arginine mutation at amino acid 505 (W505R) of VP1 capsid, or other mutations, as described in PCT Publication WO 2019/178412, which is incorporated in its entirety by reference herein.

The term “AAV virus” or “AAV viral particle” or “rAAV vector particle” refers to a viral particle composed of at least AAV capsid protein and an encapsidated polynucleotide.

The term “heterologous” refers to genotypically distinct origins. For example, a heterologous polynucleotide is one derived from a different species as compared to a reference species (for example a human gene inserted into a viral plasmid is a heterologous gene). A promoter removed from its native coding sequence and operatively linked to a coding sequence with which it is not naturally found linked is a heterologous promoter.

As used herein, the term “kit” may be used to describe variations of the portable, self-contained enclosure that includes at least one set of components to conduct one or more of the diagnostic or therapeutic methods of the present disclosure.

The term “carrier” refers to a diluent, adjuvant, excipient, or vehicle with which the rAAV particle or preparation, and/or rAAV vectors is administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions may also be employed as liquid carriers.

“Gene silencing” refers to the suppression of gene expression, e.g., transgene, heterologous gene and/or endogenous gene expression. Gene silencing may be mediated through processes that affect transcription and/or through processes that affect post-transcriptional mechanisms. In some embodiments, gene silencing occurs when siRNA initiates the degradation of the mRNA of a gene of interest in a sequence-specific manner via RNA interference. In some embodiments, gene silencing may be allele-specific. “Allcle-specific” gene silencing refers to the specific silencing of one allele of a gene.

As used herein “silencing element” refers to a component of an expression construct that suppresses gene expression, such as endogenous gene expression. The silencing elements of the disclosure can be used to epigenetically silence genes at both the post-transcriptional level or the pre-transcriptional level. In some embodiments, the silencing element is a short hairpin RNA (shRNA). In some embodiments, the silencing element is an siRNA. In a non-limiting example, epigenetic modulation of gene expression by siRNA silencing elements can result from siRNA mediated modification of chromatin structure or methylation pattern to alter gene expression.

“Knock-down,” “knock-down technology” refers to a technique of gene silencing in which the expression of a target gene is reduced as compared to the gene expression prior to the introduction of the RNAi molecule, which can lead to the inhibition of production of the target gene product. The term “reduced” is used herein to indicate that the target gene expression is lowered by 1-100%. For example, the expression may be reduced by 1, 2, 3, 4, 5, 10, 20, 30, 40, 50, 60, 70, 80, 90, 95, 99%, or above 99%. Knock-down of gene expression can be directed by the use of dsRNAs or siRNAs. For example, “RNA interference (RNAi),” which can involve the use of siRNA, has been successfully applied to knockdown the expression of specific genes in plants, D. melanogaster, C. elegans, trypanosomes, planaria, hydra, and several vertebrate species including the mouse.

“RNA interference (RNAi)” is the process of sequence-specific, post-transcriptional gene silencing initiated by siRNA. RNAi is seen in a number of organisms such as Drosophila, nematodes, fungi and plants, and is believed to be involved in anti-viral defense, modulation of transposon activity, and regulation of gene expression. During RNAi, RNAi molecules induce degradation of target mRNA with consequent sequence-specific inhibition of gene expression.

A “small interfering” or “short interfering RNA” or siRNA is a RNA duplex of nucleotides that is targeted to a gene interest. A “RNA duplex” refers to the structure formed by the complementary pairing between two regions of a RNA molecule. siRNA is “targeted” to a gene in that the nucleotide sequence of the duplex portion of the siRNA is complementary to a nucleotide sequence of the targeted gene. In some embodiments, the length of the duplex of siRNAs is less than 30 nucleotides. In some embodiments, the duplex can be 29, 28, 27, 26, 25, 24, 23, 22, 21, 20, 19, 18, 17, 16, 15, 14, 13, 12, 11 or 10 nucleotides in length. In some embodiments, the length of the duplex is 19-25 nucleotides in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12 or 13 nucleotides in length. The hairpin structure can also contain 3′ or 5′ overhang portions. In some embodiments, the overhang is a 3′ or a 5′ overhang 0, 1, 2, 3, 4 or 5 nucleotides in length. The “sense” and “antisense” sequences can be used with or without a loop region to form siRNA molecules. As used herein, the term siRNA is meant to be equivalent to other terms used to describe nucleic acid molecules that are capable of mediating sequence specific RNAi, for example, double-stranded RNA (dsRNA), micro-RNA (miRNA), short hairpin RNA (shRNA), short interfering oligonucleotide, short interfering nucleic acid, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetic silencing. For example, siRNA molecules of the disclosure can be used to epigenetically silence genes at both the post-transcriptional level or the pre-transcriptional level. In a non-limiting example, epigenetic modulation of gene expression by siRNA molecules of the invention can result from siRNA mediated modification of chromatin structure or methylation pattern to alter gene expression. In another non-limiting example, modulation of gene expression by siRNA molecules of the disclosure can result from siRNA mediated cleavage of RNA (either coding or non-coding RNA) via RISC, or alternately, translational inhibition, as is known in the art.

The silencing element (e.g., an siRNA) can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal. A nucleic acid construct containing a silencing element may be referred to herein as a “silencing construct.”

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition, or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 90%” includes “90%.” In some embodiments, at least 95% homologous or identical includes 96%, 97%, 98%, 99%, and 100% homologous or identical to the reference sequence. In addition, when a sequence is disclosed as “comprising” a nucleotide or amino acid sequence, such a reference shall also include, unless otherwise indicated, that the sequence “consists of” or “consists essentially of” the recited sequence. Likewise, when a composition is disclosed as “comprising” a feature, such a reference shall also include, unless otherwise indicated, that the composition “consists of” or “consists essentially of” the recited feature.

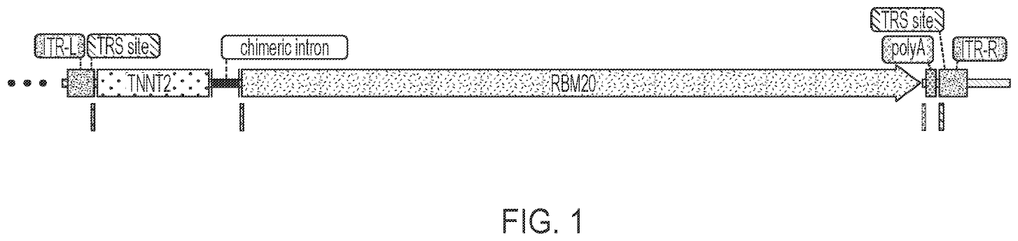

Sequence Listing Construct 1 (pTR-TNNT2-RBM20; FIG. 1)

| SEQ | Elements | |
|---|---|---|
| ID: | (5′->3′) | Nucleotide (Nt) sequence |
| 1 | 5′ ITR | TTGGCCACTCCCTCTCTGCGCGCTCGCT |
| | (ITR-L) | CGCTCACTGAGGCCGGGCGACCAAAGG |
| | | TCGCCCGACGCCCGGGCTTTGCCCGGG |
| | | CGGCCTCAGTGAGCGAGCGAGCGCGCA |
| | | GAGAGGGAGTGGCCAACTCCATCACTA |
| | | GGGGTTCCT |
| 2 | TNNT2 | GTCATGGAGAAGACCCACCTTGCAGAT |
| | promoter | GTCCTCACTGGGGCTGGCAGAGCCGGC |
| | | AACCTGCCTAAGGCTGCTCAGTCCATT |
| | | AGGAGCCAGTAGCCTGGAAGATGTCTT |
| | | TACCCCCAGCATCAGTTCAAGTGGAGC |
| | | AGCACATAACTCTTGCCCTCTGCCTTCC |
| | | AAGATTCTGGTGCTGAGACTTATGGAG |
| | | TGTCTTGGAGGTTGCCTTCTGCCCCCCA |
| | | ACCCTGCTCCCAGCTGGCCCTCCCAGG |
| | | CCTGGGTTGCTGGCCTCTGCTTTATCAG |
| | | GATTCTCAAGAGGGACAGCTGGTTTAT |
| | | GTTGCATGACTGTTCCCTGCATATCTGC |
| | | TCTGGTTTTAAATAGCTTATCTGAGCAG |
| | | CTGGAGGACCACATGGGCTTATATGGC |
| | | GTGGGGTACATGATCCTGTAGCCTTGT |
| | | CCCTGGCACCTGCCAAAATAGCAGCCA |
| | | ACACCCCCCACCCCCACCGCCATCCCC |
| | | CTGCCCCACCCGTCCCCTGTCGCACATT |
| | | CCTCCCTCCGCAGGGCTGGCTCACCAG |
| | | GCCCCAGCCCACATGCCTGCTTAAAGC |
| | | CCTCTCCATCCTCTGCCTCACCCAGTCC |
| | | CCGCTGAGACTGAGCAGACGCCTCCA |
| 3 | chimeric | CAGGTAAGTATCAAGGTTACAAGACAG |
| | intron | GTTTAAGGAGACCAATAGAAACTGGGC |
| | (with | TTGTCGAGACAGAGGGCCGGCCAAGA |
| | FseI | CTCTTGCGTTTCTGATAGGCACCTATTG |
| | site in | GTCTTACTGACATCCACTTTGCCTTTCT |
| | bold | CTCCACAGGGT |
| | underline) | |
| 4 | ACC65I | GGTACC |
| | endonucle- | |
| | ase site | |
| 5 | RBM20 cDNA | ATGGTGCTGGCAGCAGCCATGAGCCAG |
| | | GACGCGGACCCCAGCGGTCCGGAGCA |
| | | GCCGGACAGAGTTGCCTGCAGTGTGCC |
| | | TGGTGCCCGGGCGTCCCCGGCACCCTC |
| | | CGGCCCGCGAGGGATGCAGCAGCCGCC |
| | | GCCGCCGCCCCAGCCACCGCCCCCGCC |
| | | CCAAGCCGGCCTACCCCAGATCATCCA |
| | | AAATGCCGCCAAGCTCCTGGACAAGAA |
| | | CCCATTCTCGGTCAGTAACCCGAACCC |
| | | TCTGCTTCCTTCACCTGCCAGTCTCCAG |
| | | CTGGCTCAACTGCAGGCCCAGCTCACC |
| | | CTCCACCGGCTGAAGCTGGCACAGACA |
| | | GCTGTCACCAACAACACTGCAGCCGCC |
| | | ACAGTCCTGAACCAAGTCCTCTCCAAA |
| | | GTGGCCATGTCCCAGCCTCTCTTCAATC |
| | | AACTGAGGCATCCGTCTGTGATCACTG |
| | | GCCCCCACGGCCATGCTGGGGTTCCCC |
| | | AACATGCTGCAGCCATACCCAGTACCC |
| | | GGTTTCCCTCTAATGCAATTGCCTTTTC |
| | | ACCCCCCAGCCAGACACGAGGCCCCGG |
| | | ACCCTCCATGAACCTTCCCAACCAGCC |
| | | ACCCAGTGCCATGGTGATGCATCCTTT |
| | | CACTGGGGTAATGCCTCAGACCCCTGG |
| | | CCAGCCAGCAGTCATCTTGGGCATTGG |
| | | CAAGACTGGGCCTGCTCCAGCTACAGC |
| | | AGGATTCTATGAGTATGGCAAAGCCAG |
| | | CTCTGGCCAGACATATGGCCCTGAAAC |
| | | AGATGGTCAGCCTGGCTTCCTGCCATC |
| | | CTCGGCCTCAACCTCGGGCAGTGTGAC |
| | | CTATGAAGGGCACTACAGCCACACAGG |
| | | GCAGGATGGTCAAGCTGCCTTTTCCAA |
| | | AGATTTTTACGGACCCAACTCCCAAGG |
| | | TTCACATGTGGCCAGCGGATTTCCAGC |
| | | TGAGCAGGCTGGGGGCCTGAAAAGTGA |
| | | GGTCGGGCCACTGCTGCAGGGCACAAA |
| | | CAGCCAATGGGAGAGCCCCCATGGATT |
| | | CTCGGGCCAAAGCAAGCCTGATCTCAC |
| | | AGCAGGTCCCATGTGGCCTCCACCCCA |
| | | CAACCAGCCCTATGAGCTGTACGACCC |
| | | CGAGGAACCAACCTCAGACAGGACAC |
| | | CTCCTTCCTTCGGGGGTCGGCTTAACA |
| | | ACAGCAAACAGGGTTTTATCGGTGCTG |
| | | GGCGGAGGGCCAAGGAGGACCAGGCG |
| | | TTGCTATCTGTGCGGCCTCTGCAGGCTC |
| | | ATGAGCTGAACGACTTTCACGGTGTGG |
| | | CCCCCCTCCACTTGCCGCATATCTGTAG |
| | | CATCTGTGACAAGAAGGTGTTTGATTT |
| | | GAAGGACTGGGAGCTGCATGTGAAAG |
| | | GGAAGCTGCACGCTCAGAAATGCCTGG |
| | | TCTTCTCTGAAAATGCTGGCATCCGGTG |
| | | TATACTTGGTTCGGCAGAGGGAACATT |
| | | GTGTGCTTCTCCCAACAGCACAGCTGT |
| | | TTATAACCCTGCTGGGAATGAAGATTA |
| | | TGCCTCAAATCTTGGAACATCATACGT |
| | | GCCCATTCCAGCAAGGTCATTCACTCA |
| | | GTCAAGCCCCACATTTCCTTTGGCTTCT |
| | | GTGGGGACAACTTTTGCACAGCGGAAA |
| | | GGGGCTGGCCGTGTGGTGCACATCTGC |
| | | AATCTCCCTGAAGGAAGCTGCACTGAG |
| | | AATGACGTCATTAACCTGGGGCTGCCC |
| | | TTTGGAAAGGTCACTAATTACATCCTC |
| | | ATGAAATCGACTAATCAGGCCTTTTTA |
| | | GAGATGGCTTACACAGAAGCTGCACAG |
| | | GCCATGGTCCAGTATTATCAAGAAAAA |
| | | TCTGCTGTGATCAATGGTGAGAAGTTG |
| | | CTCATTCGGATGTCCAAGAGATACAAG |
| | | GAATTGCAGCTCAAGAAACCCGGGAA |
| | | GGCCGTGGCTGCCATCATCCAGGACAT |
| | | CCATTCCCAGAGGGAGAGGGACATGTT |
| | | CCGGGAAGCAGACAGATATGGCCCAG |
| | | AAAGGCCGCGGTCTCGTAGTCCGGTGA |
| | | GCCGGTCACTCTCCCCGAGGTCCCACA |
| | | CTCCCAGCTTCACCTCCTGCAGCTCTTC |
| | | CCACAGCCCTCCGGGCCCCTCCCGGGC |
| | | TGACTGGGGCAATGGCCGGGACTCCTG |
| | | GGAGCACTCTCCCTATGCCAGGAGGGA |
| | | GGAAGAGCGAGACCCGGCTCCCTGGA |
| | | GGGACAACGGAGATGACAAGAGGGAC |
| | | AGGATGGACCCCTGGGCACATGATCGC |
| | | AAACACCACCCCCGGCAACTGGACAAG |
| | | GCTGAGTTGGACGAGCGACCAGAAGG |
| | | AGGGAGGCCCCACCGGGAGAAGTACC |
| | | CGAGATCTGGGTCTCCCAACCTGCCCC |
| | | ACTCTGTGTCCAGCTACAAAAGCCGTG |
| | | AAGACGGCTACTACCGGAAAGAGCCC |
| | | AAAGCCAAGTGGGACAAGTATCTGAAG |
| | | CAGCAGCAGGATGCCCCCGGGAGGTCC |
| | | AGGAGGAAAGACGAGGCCAGGCTGCG |
| | | GGAAAGCAGACACCCCCATCCGGATGA |
| | | CTCAGGCAAGGAAGATGGGCTGGGGC |
| | | CAAAGGTCACTAGGGCCCCTGAGGGCG |
| | | CCAAGGCCAAGCAGAATGAGAAAAAT |
| | | AAAACCAAGAGAACTGATAGAGACCA |
| | | AGAAGGAGCTGATGATAGAAAAGAAA |
| | | ACACAATGGCAGAGAATGAGGCTGGA |
| | | AAAGAGGAACAGGAGGGCATGGAAGA |
| | | AAGCCCTCAATCAGTGGGCAGACAGGA |
| | | GAAAGAAGCAGAGTTCTCTGATCCGGA |
| | | AAACACAAGGACAAAGAAGGAACAAG |
| | | ATTGGGAGAGTGAAAGTGAGGCAGAG |
| | | GGGGAGAGCTGGTATCCCACTAACATG |
| | | GAGGAGCTGGTGACAGTGGACGAGGTT |
| | | GGGGAAGAAGAAGATTTTATCGTGGAA |
| | | CCAGACATCCCAGAGCTGGAAGAAATT |
| | | GTGCCCATTGACCAGAAAGACAAAATT |
| | | TGCCCAGAAACATGTCTGTGTGTGACA |
| | | ACCACCTTAGACTTAGACCTGGCCCAG |
| | | GATTTCCCCAAGGAAGGAGTCAAGGCC |
| | | GTAGGGAATGGGGCTGCAGAAATCAGC |
| | | CTCAAGTCACCCAGAGAACTGCCCTCT |
| | | GCTTCCACAAGCTGTCCCAGTGACATG |
| | | GACGTGGAAATGCCTGGCCTAAATCTG |
| | | GATGCTGAGCGGAAGCCAGCTGAAAGT |
| | | GAGACAGGCCTCTCCCTGGAGGATTCA |
| | | GATTGCTACGAGAAGGAGGCAAAGGG |
| | | AGTGGAGAGCTCAGATGTTCATCCAGC |
| | | CCCTACAGTCCAGCAAATGTCTTCCCCT |
| | | AAGCCAGCAGAGGAGAGGGCCCGGCA |
| | | GCCAAGCCCATTTGTGGATGATTGCAA |
| | | GACCAGGGGGACCCCCGAAGATGGGG |
| | | CTTGTGAAGGCAGCCCCCTGGAGGAGA |
| | | AAGCCAGCCCCCCCATCGAAACTGACC |
| | | TCCAAAACCAAGCCTGCCAAGAAGTGT |
| | | TGACCCCGGAAAACTCCAGGTACGTGG |
| | | AAATGAAATCTCTGGAGGTGAGGTCAC |
| | | CAGAGTACACTGAAGTGGAACTGAAAC |
| | | AGCCCCTTTCTTTGCCCTCTTGGGAACC |
| | | AGAGGATGTGTTCAGTGAACTTAGCAT |
| | | TCCTCTAGGGGTGGAGTTCGTGGTTCCC |
| | | AGGACTGGCTTTTATTGCAAGCTGTGT |
| | | GGGCTGTTCTACACGAGCGAGGAGACA |
| | | GCAAAGATGAGCCACTGCCGCAGCGCT |
| | | GTCCACTACAGGAACTTACAGAAATAT |
| | | TTGTCCCAGCTGGCCGAGGAGGGCCTC |
| | | AAGGAGACCGAGGGGGCAGATAGCCC |
| | | GAGGCCAGAGGACAGCGGAATCGTGC |
| | | CACGCTTCGAAAGGAAAAAGCTCTGA |
| 6 | polyA | AATAAAAGATCCTTATTTTCATTGGATC |
| | | TGTGTGTTGGTTTTTTGTGTG |
| 7 | 3′ ITR | AGGAACCCCTAGTGATGGAGTTGGCCA |
| | (ITR-R) | CTCCCTCTCTGCGCGCTCGCTCGCTCAC |
| | | TGAGGCCGGGCGACCAAAGGTCGCCCG |
| | | ACGCCCGGGCTTTGCCCGGGCGGCCTC |
| | | AGTGAGCGAGCGAGCGCGCAGAGAGG |
| | | GAGTGGCCAA |

### Sequence Listing—Proteins

| | Elements | |
|---|---|---|
| SEQ | (N-term.- | |
| ID: | >C-term.) | Protein Sequence |
| 8 | RBM20 | MVLAAAMSQDADPSGPEQPDRVACSVPGARASP |
| | | APSGPRGMQQPPPPPQPPPPPQAGLPQIIQNAA |
| | | KLLDKNPFSVSNPNPLLPSPASLQLAQLQAQLT |
| | | LHRLKLAQTAVINNTAAATVLNQVLSKVAMSQP |
| | | LFNQLRHPSVITGPHGHAGVPQHAAAIPSTRFP |
| | | SNAIAFSPPSQTRGPGPSMNLPNQPPSAMVMHP |
| | | FTGVMPQTPGQPAVILGIGKTGPAPATAGFYEY |
| | | GKASSGQTYGPETDGQPGFLPSSASTSGSVTYE |
| | | GHYSHTGQDGQAAFSKDFYGPNSQGSHVASGFP |
| | | AEQAGGLKSEVGPLLQGTNSQWESPHGFSGQSK |
| | | PDLTAGPMWPPPHNQPYELYDPEEPTSDRTPPS |
| | | FGGRLNNSKQGFIGAGRRAKEDQALLSVRPLQA |
| | | HELNDFHGVAPLHLPHICSICDKKVEDLKDWEL |
| | | HVKGKLHAQKCLVFSENAGIRCILGSAEGTLCA |
| | | SPNSTAVYNPAGNEDYASNLGTSYVPIPARSFT |
| | | QSSPTFPLASVGTTFAQRKGAGRVVHICNLPEG |
| | | SCTENDVINLGLPFGKVTNYILMKSTNQAFLEM |
| | | AYTEAAQAMVQYYQEKSAVINGEKLLIRMSKRY |
| | | KELQLKKPGKAVAAIIQDIHSQRERDMFREADR |
| | | YGPERPRSRSPVSRSLSPRSHTPSFTSCSSSHS |
| | | PPGPSRADWGNGRDSWEHSPYARREEERDPAPW |
| | | RDNGDDKRDRMDPWAHDRKHHPRQLDKAELDER |
| | | PEGGRPHREKYPRSGSPNLPHSVSSYKSREDGY |
| | | YRKEPKAKWDKYLKQQQDAPGRSRRKDEARLRE |
| | | SRHPHPDDSGKEDGLGPKVTRAPEGAKAKQNEK |
| | | NKTKRTDRDQEGADDRKENTMAENEAGKEEQEG |
| | | MEESPQSVGRQEKEAEFSDPENTRTKKEQDWES |
| | | ESEAEGESWYPTNMEELVTVDEVGEEEDFIVEP |
| | | DIPELEEIVPIDQKDKICPETCLCVTTTLDLDL |
| | | AQDFPKEGVKAVGNGAAEISLKSPRELPSASTS |
| | | CPSDMDVEMPGLNLDAERKPAESETGLSLEDSD |
| | | CYEKEAKGVESSDVHPAPTVQQMSSPKPAEERA |
| | | RQPSPFVDDCKTRGTPEDGACEGSPLEEKASPP |
| | | IETDLQNQACQEVLTPENSRYVEMKSLEVRSPE |
| | | YTEVELKQPLSLPSWEPEDVFSELSIPLGVEFV |
| | | VPRTGFYCKLCGLFYTSEETAKMSHCRSAVHYR |
| | | NLQKYLSQLAEEGLKETEGADSPRPEDSGIVPR |
| | | FERKKL |
| 11 | Rh74 VP1 | MAADGYLPDWLEDNLSEGIREWWDLKPGAPKPK |
| | (VP2, | ANQQKQDNGRGLVLPGYKYLGPFNGLDKGEPVN |
| | VP3) | AADAAALEHDKAYDQQLQAGDNPYLRYNHADAE |
| | | FQERLQEDTSFGGNLGRAVFQAKKRVLEPLGLV |
| | | ESPVKTAPGKKRPVEPSPQRSPDSSTGIGKKGQ |
| | | QPAKKRLNFGQTGDSESVPDPQPIGEPPAGPSG |
| | | LGSGTMAAGGGAPMADNNEGADGVGSSSGNWHC |
| | | DSTWLGDRVITTSTRTWALPTYNNHLYKQISNG |
| | | TSGGSTNDNTYFGYSTPWGYFDFNRFHCHFSPR |
| | | DWQRLINNNWGFRPKRLNFKLFNIQVKEVTQNE |
| | | GTKTIANNLTSTIQVFTDSEYQLPYVLGSAHQG |
| | | CLPPFPADVFMIPQYGYLTLNNGSQAVGRSSFY |
| | | CLEYFPSQMLRTGNNFEFSYNFEDVPFHSSYAH |
| | | SQSLDRLMNPLIDQYLYYLSRTQSTGGTAGTQQ |
| | | LLFSQAGPNNMSAQAKNWLPGPCYRQQRVSTTL |
| | | SQNNNSNFAWTGATKYHLNGRDSLVNPGVAMAT |
| | | HKDDEERFFPSSGVLMFGKQGAGKDNVDYSSVM |
| | | LTSEEEIKTTNPVATEQYGVVADNLQQQNAAPI |
| | | VGAVNSQGALPGMVWQNRDVYLQGPIWAKIPHT |
| | | DGNFHPSPLMGGFGLKHPPPQILIKNTPVPADP |
| | | PTTFNQAKLASFITQYSTGQVSVEIEWELQKEN |
| | | SKRWNPEIQYTSNYYKSTNVDFAVNTEGTYSEP |
| | | RPIGTRYLTRNL |
| 12 | AAV9 VP1 | MAADGYLPDWLEDNLSEGIREWWALKPGAPQPK |
| | | ANQQHQDNARGLVLPGYKYLGPGNGLDKGEPVN |
| | | AADAAALEHDKAYDQQLKAGDNPYLKYNHADAE |
| | | FQERLKEDTSFGGNLGRAVFQAKKRLLEPLGLV |
| | | EEAAKTAPGKKRPVEQSPQEPDSSAGIGKSGAQ |
| | | PAKKRLNFGQTGDTESVPDPQPIGEPPAAPSGV |
| | | GSLTMASGGGAPVADNNEGADGVGSSSGNWHCD |
| | | SQWLGDRVITTSTRTWALPTYNNHLYKQISNST |
| | | SGGSSNDNAYFGYSTPWGYFDFNRFHCHFSPRD |
| | | WQRLINNNWGFRPKRLNFKLFNIQVKEVTDNNG |
| | | VKTIANNLTSTVQVFTDSDYQLPYVLGSAHEGC |
| | | LPPFPADVFMIPQYGYLTLNDGSQAVGRSSFYC |
| | | LEYFPSQMLRTGNNFQFSYEFENVPFHSSYAHS |
| | | QSLDRLMNPLIDQYLYYLSKTINGSGQNQQTLK |
| | | FSVAGPSNMAVQGRNYIPGPSYRQQRVSTTVTQ |
| | | NNNSEFAWPGASSWALNGRNSLMNPGPAMASHK |
| | | EGEDRFFPLSGSLIFGKQGTGRDNVDADKVMIT |
| | | NEEEIKTTNPVATESYGQVATNHQSAQAQAQTG |
| | | WVQNQGILPGMVWQDRDVYLQGPIWAIPHTDGN |
| | | FHPSPLMGGFGMKHPPPQILIKNTPVPADPPTA |
| | | FNKDKLNSFITQYSTGQVSVEIEWELQKENSKR |
| | | WNPEIQYTSNYYKSNNVEFAVNTEGVYSEPRPI |
| | | GTRYLTRNL |

### Sequence Listing—Additional Sequences

| | Ele- | |
|---|---|---|
| SEQ | ments | |
| ID: | (5′->3′) | Nt Sequence |
| 9 | αMHC | CCTTCAGATTAAAAATAACTAAGGTAAGGGCCATGTG |
| | | GGTAGGGGAGGTGGTGTGAGACGGTCCTGTCTCTCCT |
| | | CTATCTGCCCATCGGCCCTTTGGGGAGGAGGAATGTG |
| | | CCCAAGGACTAAAAAAAGGCCCTGGAGCCAGAGGGG |
| | | CGAGGGCAGCAGACCTTTCATGGGCAAACCTCAGGG |
| | | CTGCTGTC |
| 10 | RH74 | ATGGCTGCCGATGGTTATCTTCCAGATTGGCTCGAGG |
| | VP1, | ACAACCTCTCTGAGGGCATTCGCGAGTGGTGGGACCT |
| | VP2, | GAAACCTGGAGCCCCGAAACCCAAAGCCAACCAGCA |
| | VP3 | AAAGCAGGACAACGGCCGGGGTCTGGTGCTTCCTGG |
| | | CTACAAGTACCTCGGACCCTTCAACGGACTCGACAAG |
| | | GGGGAGCCCGTCAACGCGGCGGACGCAGCGGCCCTC |
| | | GAGCACGACAAGGCCTACGACCAGCAGCTCCAAGCG |
| | | GGTGACAATCCGTACCTGCGGTATAATCACGCCGACG |
| | | CCGAGTTTCAGGAGCGTCTGCAAGAAGATACGTCTTT |
| | | TGGGGGCAACCTCGGGCGCGCAGTCTTCCAGGCCAA |
| | | AAAGCGGGTTCTCGAACCTCTGGGCCTGGTTGAATCG |
| | | CCGGTTAAGACGGCTCCTGGAAAGAAGAGACCGGTA |
| | | GAGCCATCACCCCAGCGCTCTCCAGACTCCTCTACGG |
| | | GCATCGGCAAGAAAGGCCAGCAGCCCGCAAAAAAGA |
| | | GACTCAATTTTGGGCAGACTGGCGACTCAGAGTCAGT |
| | | CCCCGACCCTCAACCAATCGGAGAACCACCAGCAGG |
| | | CCCCTCTGGTCTGGGATCTGGTACAATGGCTGCAGGC |
| | | GGTGGCGCTCCAATGGCAGACAATAACGAAGGCGCC |
| | | GACGGAGTGGGTAGTTCCTCAGGAAATTGGCATTGCG |
| | | ATTCCACATGGCTGGGCGACAGAGTCATCACCACCAG |
| | | CACCCGCACCTGGGCCCTGCCCACCTACAACAACCAC |
| | | CTCTACAAGCAAATCTCCAACGGGACCTCGGGAGGA |
| | | AGCACCAACGACAACACCTACTTCGGCTACAGCACCC |
| | | CCTGGGGGTATTTTGACTTCAACAGATTCCACTGCCA |
| | | CTTTTCACCACGTGACTGGCAGCGACTCATCAACAAC |
| | | AACTGGGGATTCCGGCCCAAGAGGCTCAACTTCAAGC |
| | | TCTTCAACATCCAAGTCAAGGAGGTCACGCAGAATGA |
| | | AGGCACCAAGACCATCGCCAATAACCTTACCAGCAC |
| | | GATTCAGGTCTTTACGGACTCGGAATACCAGCTCCCG |
| | | TACGTGCTCGGCTCGGCGCACCAGGGCTGCCTGCCTC |
| | | CGTTCCCGGCGGACGTCTTCATGATTCCTCAGTACGG |
| | | GTACCTGACTCTGAACAATGGCAGTCAGGCTGTGGGC |
| | | CGGTCGTCCTTCTACTGCCTGGAGTACTTTCCTTCTCA |
| | | AATGCTGAGAACGGGCAACAACTTTGAATTCAGCTAC |
| | | AACTTCGAGGACGTGCCCTTCCACAGCAGCTACGCGC |
| | | ACAGCCAGAGCCTGGACCGGCTGATGAACCCTCTCAT |
| | | CGACCAGTACTTGTACTACCTGTCCCGGACTCAAAGC |
| | | ACGGGCGGTACTGCAGGAACTCAGCAGTTGCTATTTT |
| | | CTCAGGCCGGGCCTAACAACATGTCGGCTCAGGCCAA |
| | | GAACTGGCTACCCGGTCCCTGCTACCGGCAGCAACGC |
| | | GTCTCCACGACACTGTCGCAGAACAACAACAGCAACT |
| | | TTGCCTGGACGGGTGCCACCAAGTATCATCTGAATGG |
| | | CAGAGACTCTCTGGTGAATCCTGGCGTTGCCATGGCT |
| | | ACCCACAAGGACGACGAAGAGCGATTTTTTCCATCCA |
| | | GCGGAGTCTTAATGTTTGGGAAACAGGGAGCTGGAA |
| | | AAGACAACGTGGACTATAGCAGCGTGATGCTAACCA |
| | | GCGAGGAAGAAATAAAGACCACCAACCCAGTGGCCA |
| | | CAGAACAGTACGGCGTGGTGGCCGATAACCTGCAAC |
| | | AGCAAAACGCCGCTCCTATTGTAGGGGCCGTCAATAG |
| | | TCAAGGAGCCTTACCTGGCATGGTGTGGCAGAACCGG |
| | | GACGTGTACCTGCAGGGTCCCATCTGGGCCAAGATTC |
| | | CTCATACGGACGGCAACTTTCATCCCTCGCCGCTGAT |
| | | GGGAGGCTTTGGACTGAAGCATCCGCCTCCTCAGATC |
| | | CTGATTAAAAACACACCTGTTCCCGCGGATCCTCCGA |
| | | CCACCTTCAATCAGGCCAAGCTGGCTTCTTTCATCAC |
| | | GCAGTACAGTACCGGCCAGGTCAGCGTGGAGATCGA |
| | | GTGGGAGCTGCAGAAGGAGAACAGCAAACGCTGGAA |
| | | CCCAGAGATTCAGTACACTTCCAACTACTACAAATCT |
| | | ACAAATGTGGACTTTGCTGTCAATACTGAGGGTACTT |
| | | ATTCCGAGCCTCGCCCCATTGGCACCCGTTACCTCAC |
| | | CCGTAATCTGTAA |

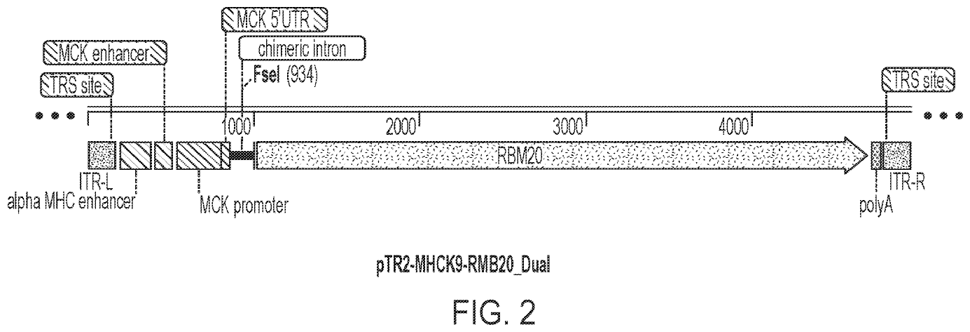

Sequence Listing Construct 2 (pTR2-MHCK9-RBM20; FIG. 2)

| SEQ | Elements | |
|---|---|---|
| ID: | (5′->3′) | Nt sequence |
| 13 | ITR-L | TTGGCCACTCCCTCTCTGCGCGCTCGCT |
| | | CGCTCACTGAGGCCGGGCGACCAAAGG |
| | | TCGCCCGACGCCCGGGCTTTGCCCGGG |
| | | CGGCCTCAGTGAGCGAGCGAGCGCGCA |
| | | GAGAGGGAGTGGCCAACTCCATCACTA |
| | | GGGGTTCCT |
| 14 | Alpha | ACCCTTCAGATTAAAAATAACTGAGGT |
| | MHC | AAGGGCCTGGGTAGGGGAGGTGGTGTG |
| | Enhancer | AGACGCTCCTGTCTCTCCTCTATCTGCC |
| | | CATCGGCCCTTTGGGGAGGAGGAATGT |
| | | GCCCAAGGACTAAAAAAAGGCCATGG |
| | | AGCCAGAGGGGCGAGGGCAACAGACC |
| | | TTTCATGGGCAAACCTTGGGGCCCTGC |
| | | TGT |
| 15 | MHCK9 | CTGCCCATGTAAGGAGGCAAGGCCTGG |
| | Enhancer | GGACACCCGAGATGCCTGGTTATAATT |
| | | AACCCAGACATGTGGCTGCCCCCCCCC |
| | | CCCCAACACCTGCTGCCTCTAAAAATA |
| | | ACC |
| 16 | MHCK9 | GTTCCCGGCGAAGGGCCAGCTGTCCCC |
| | Promoter | CGCCAGCTAGACTCAGCACTTAGTTTA |
| | | GGAACCAGTGAGCAAGTCAGCCCTTGG |
| | | GGCAGCCCATACAAGGCCATGGGGCTG |
| | | GGCAAGCTGCACGCCTGGGTCCGGGGT |
| | | GGGCACGGTGCCCGGGCAACGAGCTG |
| | | AAAGCTCATCTGCTCTCAGGGGCCCCT |
| | | CCCTGGGGACAGCCCCTCCTGGCTAGT |
| | | CACACCCTGTAGGCTCCTCTATATAAC |
| | | CCAGGGGCACAGGGGCTGCCCTC |
| 17 | MHCK9 | ACCACCACCTCCACAGCACAGACAGAC |
| | 5′ UTR | ACTCAGGAGCAGCCAG |
| 18 | chimeric | CAGGTAAGTATCAAGGTTACAAGACAG |
| | intron | GTTTAAGGAGACCAATAGAAACTGGGC |
| | (with | TTGTCGAGACAGAGGGCCGGCCAAGA |
| | FseI | CTCTTGCGTTTCTGATAGGCACCTATTG |
| | site in | GTCTTACTGACATCCACTTTGCCTTTCT |
| | bold | CTCCACAGGGT |
| | underline) | |
| 19 | RBM20 | ATGGTGCTGGCAGCAGCCATGAGCCAG |
| | | GACGCGGACCCCAGCGGTCCGGAGCA |
| | | GCCGGACAGAGTTGCCTGCAGTGTGCC |
| | | TGGTGCCCGGGCGTCCCCGGCACCCTC |
| | | CGGCCCGCGAGGGATGCAGCAGCCGCC |
| | | GCCGCCGCCCCAGCCACCGCCCCCGCC |
| | | CCAAGCCGGCCTACCCCAGATCATCCA |
| | | AAATGCCGCCAAGCTCCTGGACAAGAA |
| | | CCCATTCTCGGTCAGTAACCCGAACCC |
| | | TCTGCTTCCTTCACCTGCCAGTCTCCAG |
| | | CTGGCTCAACTGCAGGCCCAGCTCACC |
| | | CTCCACCGGCTGAAGCTGGCACAGACA |
| | | GCTGTCACCAACAACACTGCAGCCGCC |
| | | ACAGTCCTGAACCAAGTCCTCTCCAAA |
| | | GTGGCCATGTCCCAGCCTCTCTTCAATC |
| | | AACTGAGGCATCCGTCTGTGATCACTG |
| | | GCCCCCACGGCCATGCTGGGGTTCCCC |
| | | AACATGCTGCAGCCATACCCAGTACCC |
| | | GGTTTCCCTCTAATGCAATTGCCTTTTC |
| | | ACCCCCCAGCCAGACACGAGGCCCCGG |
| | | ACCCTCCATGAACCTTCCCAACCAGCC |
| | | ACCCAGTGCCATGGTGATGCATCCTTT |
| | | CACTGGGGTAATGCCTCAGACCCCTGG |
| | | CCAGCCAGCAGTCATCTTGGGCATTGG |
| | | CAAGACTGGGCCTGCTCCAGCTACAGC |
| | | AGGATTCTATGAGTATGGCAAAGCCAG |
| | | CTCTGGCCAGACATATGGCCCTGAAAC |
| | | AGATGGTCAGCCTGGCTTCCTGCCATC |
| | | CTCGGCCTCAACCTCGGGCAGTGTGAC |
| | | CTATGAAGGGCACTACAGCCACACAGG |
| | | GCAGGATGGTCAAGCTGCCTTTTCCAA |
| | | AGATTTTTACGGACCCAACTCCCAAGG |
| | | TTCACATGTGGCCAGCGGATTTCCAGC |
| | | TGAGCAGGCTGGGGGCCTGAAAAGTGA |
| | | GGTCGGGCCACTGCTGCAGGGCACAAA |
| | | CAGCCAATGGGAGAGCCCCCATGGATT |
| | | CTCGGGCCAAAGCAAGCCTGATCTCAC |
| | | AGCAGGTCCCATGTGGCCTCCACCCCA |
| | | CAACCAGCCCTATGAGCTGTACGACCC |
| | | CGAGGAACCAACCTCAGACAGGACAC |
| | | CTCCTTCCTTCGGGGGTCGGCTTAACA |
| | | ACAGCAAACAGGGTTTTATCGGTGCTG |
| | | GGCGGAGGGCCAAGGAGGACCAGGCG |
| | | TTGCTATCTGTGCGGCCTCTGCAGGCTC |
| | | ATGAGCTGAACGACTTTCACGGTGTGG |
| | | CCCCCCTCCACTTGCCGCATATCTGTAG |
| | | CATCTGTGACAAGAAGGTGTTTGATTT |
| | | GAAGGACTGGGAGCTGCATGTGAAAG |
| | | GGAAGCTGCACGCTCAGAAATGCCTGG |
| | | TCTTCTCTGAAAATGCTGGCATCCGGTG |
| | | TATACTTGGTTCGGCAGAGGGAACATT |
| | | GTGTGCTTCTCCCAACAGCACAGCTGT |
| | | TTATAACCCTGCTGGGAATGAAGATTA |
| | | TGCCTCAAATCTTGGAACATCATACGT |
| | | GCCCATTCCAGCAAGGTCATTCACTCA |
| | | GTCAAGCCCCACATTTCCTTTGGCTTCT |
| | | GTGGGGACAACTTTTGCACAGCGGAAA |
| | | GGGGCTGGCCGTGTGGTGCACATCTGC |
| | | AATCTCCCTGAAGGAAGCTGCACTGAG |
| | | AATGACGTCATTAACCTGGGGCTGCCC |
| | | TTTGGAAAGGTCACTAATTACATCCTC |
| | | ATGAAATCGACTAATCAGGCCTTTTTA |
| | | GAGATGGCTTACACAGAAGCTGCACAG |
| | | GCCATGGTCCAGTATTATCAAGAAAAA |
| | | TCTGCTGTGATCAATGGTGAGAAGTTG |
| | | CTCATTCGGATGTCCAAGAGATACAAG |
| | | GAATTGCAGCTCAAGAAACCCGGGAA |
| | | GGCCGTGGCTGCCATCATCCAGGACAT |
| | | CCATTCCCAGAGGGAGAGGGACATGTT |
| | | CCGGGAAGCAGACAGATATGGCCCAG |
| | | AAAGGCCGCGGTCTCGTAGTCCGGTGA |
| | | GCCGGTCACTCTCCCCGAGGTCCCACA |
| | | CTCCCAGCTTCACCTCCTGCAGCTCTTC |
| | | CCACAGCCCTCCGGGCCCCTCCCGGGC |
| | | TGACTGGGGCAATGGCCGGGACTCCTG |
| | | GGAGCACTCTCCCTATGCCAGGAGGGA |
| | | GGAAGAGCGAGACCCGGCTCCCTGGA |
| | | GGGACAACGGAGATGACAAGAGGGAC |
| | | AGGATGGACCCCTGGGCACATGATCGC |
| | | AAACACCACCCCCGGCAACTGGACAAG |
| | | GCTGAGTTGGACGAGCGACCAGAAGG |
| | | AGGGAGGCCCCACCGGGAGAAGTACC |
| | | CGAGATCTGGGTCTCCCAACCTGCCCC |
| | | ACTCTGTGTCCAGCTACAAAAGCCGTG |
| | | AAGACGGCTACTACCGGAAAGAGCCC |
| | | AAAGCCAAGTGGGACAAGTATCTGAAG |
| | | CAGCAGCAGGATGCCCCCGGGAGGTCC |
| | | AGGAGGAAAGACGAGGCCAGGCTGCG |
| | | GGAAAGCAGACACCCCCATCCGGATGA |
| | | CTCAGGCAAGGAAGATGGGCTGGGGC |
| | | CAAAGGTCACTAGGGCCCCTGAGGGCG |
| | | CCAAGGCCAAGCAGAATGAGAAAAAT |
| | | AAAACCAAGAGAACTGATAGAGACCA |
| | | AGAAGGAGCTGATGATAGAAAAGAAA |
| | | ACACAATGGCAGAGAATGAGGCTGGA |
| | | AAAGAGGAACAGGAGGGCATGGAAGA |
| | | AAGCCCTCAATCAGTGGGCAGACAGGA |
| | | GAAAGAAGCAGAGTTCTCTGATCCGGA |
| | | AAACACAAGGACAAAGAAGGAACAAG |
| | | ATTGGGAGAGTGAAAGTGAGGCAGAG |
| | | GGGGAGAGCTGGTATCCCACTAACATG |
| | | GAGGAGCTGGTGACAGTGGACGAGGTT |
| | | GGGGAAGAAGAAGATTTTATCGTGGAA |
| | | CCAGACATCCCAGAGCTGGAAGAAATT |
| | | GTGCCCATTGACCAGAAAGACAAAATT |
| | | TGCCCAGAAACATGTCTGTGTGTGACA |
| | | ACCACCTTAGACTTAGACCTGGCCCAG |
| | | GATTTCCCCAAGGAAGGAGTCAAGGCC |
| | | GTAGGGAATGGGGCTGCAGAAATCAGC |
| | | CTCAAGTCACCCAGAGAACTGCCCTCT |
| | | GCTTCCACAAGCTGTCCCAGTGACATG |
| | | GACGTGGAAATGCCTGGCCTAAATCTG |
| | | GATGCTGAGCGGAAGCCAGCTGAAAGT |
| | | GAGACAGGCCTCTCCCTGGAGGATTCA |
| | | GATTGCTACGAGAAGGAGGCAAAGGG |
| | | AGTGGAGAGCTCAGATGTTCATCCAGC |
| | | CCCTACAGTCCAGCAAATGTCTTCCCCT |
| | | AAGCCAGCAGAGGAGAGGGCCCGGCA |
| | | GCCAAGCCCATTTGTGGATGATTGCAA |
| | | GACCAGGGGGACCCCCGAAGATGGGG |
| | | CTTGTGAAGGCAGCCCCCTGGAGGAGA |
| | | AAGCCAGCCCCCCCATCGAAACTGACC |
| | | TCCAAAACCAAGCCTGCCAAGAAGTGT |
| | | TGACCCCGGAAAACTCCAGGTACGTGG |
| | | AAATGAAATCTCTGGAGGTGAGGTCAC |
| | | CAGAGTACACTGAAGTGGAACTGAAAC |
| | | AGCCCCTTTCTTTGCCCTCTTGGGAACC |
| | | AGAGGATGTGTTCAGTGAACTTAGCAT |
| | | TCCTCTAGGGGTGGAGTTCGTGGTTCCC |
| | | AGGACTGGCTTTTATTGCAAGCTGTGT |
| | | GGGCTGTTCTACACGAGCGAGGAGACA |
| | | GCAAAGATGAGCCACTGCCGCAGCGCT |
| | | GTCCACTACAGGAACTTACAGAAATAT |
| | | TTGTCCCAGCTGGCCGAGGAGGGCCTC |
| | | AAGGAGACCGAGGGGGCAGATAGCCC |
| | | GAGGCCAGAGGACAGCGGAATCGTGC |
| | | CACGCTTCGAAAGGAAAAAGCTCTGA |
| 20 | Poly A | AATAAAAGATCCTTATTTTCATTGGATC |
| | | TGTGTGTTGGTTTTTTGTGTG |
| 21 | ITR-R | AGGAACCCCTAGTGATGGAGTTGGCCA |
| | | CTCCCTCTCTGCGCGCTCGCTCGCTCAC |
| | | TGAGGCCGGGCGACCAAAGGTCGCCCG |
| | | ACGCCCGGGCTTTGCCCGGGCGGCCTC |
| | | AGTGAGCGAGCGAGCGCGCAGAGAGG |
| | | GAGTGGCCAA |

### Kozak Sequences

In some embodiments, described herein is a nucleic acid comprising an expression construct comprising a human RBM20 coding sequence and an enhancer element, such as a CMV enhancer, operably linked to a promoter, wherein the expression construct is flanked on each side by an inverted terminal repeat sequence. In some embodiments, described herein is a nucleic acid comprising an expression construct comprising a human RBM20 coding sequence, an enhancer element operably linked to a promoter, and a Kozak sequence, wherein the Kozak sequence enhances transgene expression in the heart, wherein the expression construct is flanked on each side by an inverted terminal repeat sequence, wherein the Kozak sequence is non-native with respect to the human RBM20 coding sequence and/or non-native to the promoter. In some embodiments, described herein is a nucleic acid comprising an expression construct comprising a human RBM20 coding sequence, an enhancer element operably linked to a promoter, and an in silico designed consensus Kozak sequence, wherein the in silico designed consensus Kozak sequence enhances transgene expression in the heart, wherein the expression construct is flanked on each side by an inverted terminal repeat sequence, wherein the Kozak sequence is non-native with respect to the human RBM20 coding sequence and the promoter. In some embodiments, described herein is a nucleic acid comprising an expression construct comprising a human RBM20 coding sequence, an enhancer element operably linked to a promoter, and a Kozak sequence, wherein the Kozak sequence enhances transgene expression in the heart, wherein the expression construct is flanked on each side by an inverted terminal repeat sequence, wherein the Kozak sequence is native with respect to the human RBM20 coding sequence and/or native to the promoter. In several embodiments, the Kozak sequence is a synthetic sequence. In some embodiments, the human RBM20 coding sequence is codon-optimized for expression in human cells. In some embodiments, the promoter comprises a cardiac specific promoter. In some embodiments, the promoter is CBA (Chicken β-Actin), or a truncated chicken beta-actin (smCBA). In some embodiments, the nucleic acid is a recombinant adeno-associated virus (rAAV) vector. In some embodiments, the nucleic acid is a single-stranded or self-complementary rAAV nucleic acid vector. In some embodiments, the rAAV particle is an AAV9 particle. In some embodiments, the rAAV particle is an rh74 (or AAVrh74) particle. In some embodiments, the rAAV particle is an rh10 (or AAVrh10) particle. In some embodiments, a composition comprising a plurality of rAAV particles is provided. In some embodiments, the plurality of rAAV particles may further comprise a pharmaceutically acceptable carrier. In some embodiments, the rh74 particle comprises at least one capsid protein encoded by a polynucleotide having at least 85%, at least 90%, at least 95%, at least 96%, at least 97%, at least 98% or at least 99% sequence identity to the nucleotide sequence set forth as SEQ ID NO: 10, or a portion of SEQ ID NO: 10 (for example, SEQ ID NO: 10 encodes the rh74 VP1, VP2, and VP3 proteins-thus, in several embodiments, an rh74 particle according to embodiments disclosed herein comprises at least one capsid protein encoded by a polynucleotide having at least 85%, at least 90%, at least 95%, at least 96%, at least 97%, at least 98% or at least 99% sequence identity to a subpart of the nucleotide sequence of SEQ ID NO: 10). In some embodiments, the rh74 particle comprises an amino acid sequence having at least about 85%, at least 90%, at least 95%, at least 96%, at least 97%, at least 98% or at least 99% sequence identity to the amino acid sequence set forth as SEQ ID NO: 11, or a portion of SEQ ID NO: 11 (for example, SEQ ID NO: 11 is the amino acid sequence of rh74 VP1, VP2, and VP3 proteins-thus, in several embodiments, an rh74 particle according to embodiments disclosed herein comprises at least one capsid protein having at least 85%, at least 90%, at least 95%, at least 96%, at least 97%, at least 98% or at least 99% sequence identity to a subpart of the amino acid sequence of SEQ ID NO: 11). In some embodiments, the AAV9 particle comprises an amino acid sequence having at least about 85%, at least 90%, at least 95%, at least 96%, at least 97%, at least 98% or at least 99% sequence identity to the amino acid sequence set forth as SEQ ID NO: 12.

### The Transgene

A transgene may be employed to correct, reduce, eliminate, or otherwise ameliorate gene deficiencies, which may include deficiencies in which normal genes are expressed at less than normal levels, are expressed at normal or near-normal levels but having a gene product with abnormal activity, or deficiencies in which the functional gene product is not expressed. In several embodiments, the transgene sequence encodes a therapeutic protein or polypeptide which is to be expressed in a host cell. Embodiments of the present disclosure also include using multiple transgenes.

RNA binding motif protein 20 is encoded by the RBM20 gene. Mutations in or perturbations in the function of RBM20 are known to be causative of DCM (Dilated Cardiomyopathy). RBM20 is a major regulator of heart-specific alternative splicing of the TTN gene, which is found to be most frequently mutated in patients with idiopathic DCM (approximately 20-25%). The TTN gene has the largest number of exons (364 in humans) and titin, a sarcomeric protein encoded by the TTN gene, is the largest known protein in mammals. In an RBM20 mutant rat strain lacking nearly all the RBM20 exons, the shortest cardiac titin isoform N2B is not expressed. Therefore, RBM20 is a key regulator of TTN pre-mRNA processing in the heart and may cause DCM phenotypes through altered splicing of the RBM20-regulated genes. Missense mutations in a highly conserved RSRSP stretch, within an arginine/serine (RS)-rich region and not in the RNA binding domains are the most frequent disease alleles. In some embodiments of the disclosed rAAV vectors, the transgene is RBM20 cDNA, such as human RBM20 cDNA. In some embodiments, the transgene is an RBM20 coding sequence that has been codon-optimized for expression in a mammalian cell. In some embodiments, the transgene is an RBM20 coding sequence that has been codon optimized for expression in human cells.

In some embodiments, any of the disclosed rAAV vectors contain multiple transgenes. In some embodiments, the rAAV vector discloses two transgenes.

### Regulatory Elements

In some embodiments, the rAAV vector comprises one or more regions comprising a sequence that facilitates expression of the heterologous nucleic acid, e.g., expression regulatory sequences operatively linked to the heterologous nucleic acid. A promoter drives transcription of the nucleic acid sequence that it regulates, thus, it is typically located at or near the transcriptional start site of a gene. A promoter may have, for example, a length of 100 to 1000 nucleotides. In some embodiments, a promoter is operably linked to a nucleic acid, or a sequence of a nucleic acid (nucleotide sequence). A promoter is considered to be “operably linked” to a sequence of nucleic acid that it regulates when the promoter is in a correct functional location and orientation relative to the sequence such that the promoter regulates (e.g., to control (“drive”) transcriptional initiation and/or expression of) that sequence. Numerous such sequences are known in the art.

Promoters that may be used in accordance with the present disclosure may comprise any promoter that can drive the expression of the transgenes in the heart of the subject. In some embodiments, the promoter may be a tissue-specific promoter. A “tissue-specific promoter”, as used herein, refers to promoters that can only function in a specific type of tissue, e.g., the heart. Thus, a “tissue-specific promoter” is not able to drive the expression of the transgenes in other types of tissues. In some embodiments, the promoter that may be used in accordance with the present disclosure is a cardiac-restricted promoter. Non-limiting examples of tissue-specific promoters and/or regulatory elements that may be used include (1) desmin, creatine kinase, myogenin, alpha myosin heavy chain, and natriuretic peptide, specific for muscle cells, and (2) albumin, alpha-1-antitrypsin, hepatitis B virus core protein promoters, specific for liver cells. In some embodiments, the promoter is a muscle creatine kinase promoter, such as muscle and heart-specific promoter MHCK9. Non-limiting examples of cardiac-restricted promoter selected from cardiac troponin C, cardiac troponin I, and cardiac troponin T (cTnT). In treating cardiomyopathies as provided for herein, cardiac-restricted promoters are advantageous at least due to the reduced possibility of off-target expression of the transgene(s), thereby effectively increasing the delivered dose to the heart and enhancing therapy. Non-limiting examples of expression regulatory sequences include promoters, insulators, silencers, response elements, introns, enhancers, initiation sites, termination signals, and poly(A) tails. Any combination of such regulatory sequences is contemplated herein (e.g., a promoter and an enhancer).

Alternatively, the promoter may be, without limitation, a promoter from one of the following genes: α-myosin heavy chain gene, 6-myosin heavy chain gene, myosin light chain 2v (MLC-2v) gene, myosin light chain 2a gene, CARP gene, cardiac α-actin gene, cardiac m2 muscarinic acetylcholine gene, atrial natriuretic factor gene (ANF), cardiac sarcoplasmic reticulum Ca-ATPase gene, skeletal α-actin gene; or an artificial cardiac promoter derived from MLC-2v gene.

To achieve appropriate expression levels of the nucleic acid, protein, or polypeptide of interest, any of a number of promoters suitable for use in the selected host cell may be employed. The promoter may be, for example, a constitutive promoter, tissue-specific promoter, inducible promoter, or a synthetic promoter. For example, constitutive promoters of different strengths can be used. An rAAV vector described herein may include one or more constitutive promoters, such as viral promoters or promoters from mammalian genes that are generally active in promoting transcription. Non-limiting examples of constitutive viral promoters include the Herpes Simplex virus (HSV), thymidine kinase (TK), Rous Sarcoma Virus (RSV), Simian Virus 40 (SV40), Mouse Mammary Tumor Virus (MMTV), Ad E1A and cytomegalovirus (CMV) promoters. Non-limiting examples of non-viral constitutive promoters include various housekeeping gene promoters, as exemplified by the β-actin promoter, including the chicken β-actin promoter (CBA).

Inducible promoters and/or regulatory elements may also be contemplated for achieving appropriate expression levels of the protein or polypeptide of interest. Non-limiting examples of suitable inducible promoters include those from genes such as cytochrome P450 genes, heat shock protein genes, metallothionein genes, and hormone-inducible genes, such as the estrogen gene promoter. Another example of an inducible promoter is the tetVP16 promoter that is responsive to tetracycline.

Synthetic promoters are also contemplated herein. A synthetic promoter may comprise, for example, regions of known promoters, regulatory elements, transcription factor binding sites, enhancer elements, repressor elements, and the like.

Enhancer elements can function in combination with other regulatory elements to increase the expression of a transgene. In several embodiments, the enhancer elements are upstream (positioned 5′) of the transgene. Non-limiting embodiments of enhancer elements include nucleotide sequences comprising, for example, a 100 base pair element from Simian virus 40 (SV40 late 2×USE), a 35 base pair element from Human Immunodeficiency Virus 1 (HIV-1 USE), a 39 base pair element from ground squirrel hepatitis virus (GHV USE), a 21 base pair element from adenovirus (Adenovirus L3 USE), a 21 base pair element from human prothrombin (hTHGB USE), a 53 base pair element from human C2 complement gene (hC2 USE), truncations of any of the foregoing, and combinations of the foregoing. In some embodiments, the enhancer is an MHCK9 enhancer. In some embodiments the enhancer is derived from the α-myosin heavy chain (αMHC) gene. In some embodiments the αMHC enhancer comprises a nucleic acid sequence having at least about 85%, about 90%, about 95%, about 96%, about 97%, about 98%, about 99%, or 100% sequence identity to:

(SEQ ID NO: 9)
CCTTCAGATTAAAAATAACTAAGGTAAGGGCCATGTGGGTAGGGGAGGTG
GTGTGAGACGGTCCTGTCTCTCCTCTATCTGCCCATCGGCCCTTTGGGGA
GGAGGAATGTGCCCAAGGACTAAAAAAAGGCCCTGGAGCCAGAGGGGCGA
GGGCAGCAGACCTTTCATGGGCAAACCTCAGGGCTGCTGTC;
or to SEQ ID NO: 14.

Non-limiting polyadenylation signals include nucleotide sequences comprising, for example, a 624 base pair polyadenylation signal from human growth hormone (hGH), a 135 base pair polyadenylation signal from simian virus 40 (sV40 late), a 49 base pair synthetic polyadenylation signal from rabbit beta-globin (SPA), a 250 base pair polyadenylation signal from bovine growth hormone (bGH), truncations of any of the foregoing, and combinations of the foregoing.

In some embodiments of the disclosed rAAV vectors, the two or more transgenes are operably controlled by a single promoter. In some embodiments, each of the two or more transgenes are operably controlled by a distinct promoter.

In some embodiments, the rAAV vectors of the present disclosure further comprise an Internal Ribosome Entry Site (IRES). An IRES is a nucleotide sequence that allows for translation initiation in the middle of a messenger RNA (mRNA) sequence as part of the greater process of protein synthesis. Usually, in eukaryotes, translation can be initiated only at the 5′ end of the mRNA molecule, since 5′ cap recognition is required for the assembly of the initiation complex. In some embodiments, the IRES is located between the transgenes.

In such embodiments, the proteins encoded by different transgenes are translated individually (i.e., versus translated as a fusion protein). In some embodiments, the rAAV vectors of the present disclosure comprise at least, in order from 5′ to 3′, a first adeno-associated virus (AAV) inverted terminal repeat (ITR) sequence, a promoter operably linked to a first transgene, an IRES operably linked to a second transgene, a polyadenylation signal, and a second AAV inverted terminal repeat (ITR) sequence. In some embodiments, the rAAV vectors of the present disclosure comprise in order from 5′ to 3′, a first adeno-associated virus (AAV) inverted terminal repeat (ITR) sequence, a promoter operably linked to an RBM20 cDNA transgene, an IRES operably linked to a second transgene, a polyadenylation signal, and a second AAV inverted terminal repeat (ITR) sequence.

In some embodiments, the rAAV vectors of the present disclosure further comprise a polyadenylation (pA) signal.

### Expression Cassette

The expression cassette is composed of, at a minimum, a transgene and its regulatory sequences. Where the cassette is designed to be expressed from a rAAV, the expression cassette further contains 5′ and 3′ AAV ITRs. These ITRs may be full-length, or one or both of the ITRs may be truncated. In one embodiment, the rAAV is pseudotyed, i.e., the AAV capsid is from a different source AAV than that the

AAV which provides the ITRs. In one embodiment, the ITRs of AAV serotype 2 are used. In additional embodiments, the ITRs of AAV serotype 1 are used. However, ITRs from other suitable sources may be selected.

FIG. 1 depicts an embodiment of a construct described herein. At the 5′ end, an AAV ITR, TRS (transcription regulatory sequence) site, and TNNT2 promoter are present. A chimeric intron follows, wherein a silencing element is present, the silencing element encoding an shRNA. Following the promoter and silencing element, the RBM20 transgene is depicted. The construct further includes a polyadenylated site, and TRS site following the RBM20 transgene. Within the structural sequences described in the aforementioned construct, at least one or a plurality of spacer sequences may be inserted at any point within the construct. Additionally, any number of promoter or regulatory sequences may comprise a construct to alter or change the expression of RBM20.

FIG. 2 depicts an embodiment of a construct described herein. At the 5′ end, an AAV ITR, TRS (transcription regulatory sequence) site, alpha MHC, MHCK9 enhancer, and MHCK9 promoter are present. A chimeric intron follows. Following the promoter, the RBM20 transgene is depicted. The construct further includes a polyadenylated site, and TRS site following the RBM20 transgene. Within the structural sequences described in the aforementioned construct, at least one or a plurality of spacer sequences may be inserted at any point within the construct. Additionally, any number of promoter or regulatory sequences may comprise a construct to alter or change the expression of RBM20.

### Expression Cassette—Silencing Elements

Embodiments of this disclosure can provide compositions and methods for gene silencing and modulating protein expression using small nucleic acid molecules. Examples of nucleic acid molecules include molecules active in RNA interference (RNAi molecules), short interfering RNA (siRNA), double-stranded RNA (dsRNA), micro-RNA (miRNA), or short hairpin RNA (shRNA) molecules, as well as DNA-directed RNAs (ddRNA), Piwi-interacting RNAs (piRNA), or repeat associated siRNAs (rasiRNA). Such molecules are capable of mediating RNA interference against gene expression. In some embodiments, gene silencing can target a specific defective allele. In some embodiments, the gene silenced defective allele can then be replaced by a functional copy. In some embodiments, the functional copy of a gene is codon optimized (e.g., for expression in human cells), such that dissimilarities between a defective copy and a functional copy allow for silencing only of the defective copy.

In some embodiments, the expression cassette comprises a RBM20 transgene and associated regulatory sequences, as well as a region capable of modulating endogenous RBM20 gene expression, e.g., via a shRNA expression cassette. Attenuation, or knock down of endogenous gene expression can be accomplished using nucleotide sequences coding for small nucleic acid molecules, including shRNA. In some embodiments, the expression cassette comprises a transgene coding for a functional RBM20 allele, as well as silencing elements to attenuate expression of a defective gene. In some embodiments, the silencing element is an intronic sequence within the overall construct. In some embodiments, the intronic sequence contains a restriction site. In some embodiments, the silencing element and intronic sequence can be utilized for subcloning in the expression cassette.

In some embodiments, delivery of nucleotide sequences can be separate from the vector encoding the expression cassette comprising a transgene and associated regulatory sequences. For example, two or more constructs may be co-administered, wherein at least one transgene construct comprises nucleic acid sequences encoding a functional RBM20 transgene, and wherein at least one other silencing construct comprises nucleic acid sequences for regulating endogenous RBM20 gene expression. In some embodiments, administration of an expression cassette encoding a RBM20 transgene is accompanied by, followed by, or preceded by, administration of a vector encoding a method for gene silencing or modulating RBM20 protein expression.

In some embodiments, the expression cassette comprises a RBM20 transgene and associated regulatory sequences, but does not include a region modulating endogeonous RBM20 gene expression. In some embodiments, a construct comprising the expression cassette with the functional RBM20 transgene is administered. In some embodiments, the expression of the functional RBM20 transgene is sufficient to provide therapeutic benefits to a subject. In some embodiments, the expression of the functional RBM20 transgene provides gain of RNA binding motif 20 function to a subject.

### The Vector

Further provided herein are rAAV viral particles or rAAV preparations containing such particles. In several embodiments, rAAV particles comprise a viral capsid and one or more transgenes as described herein, which is encapsidated by the viral capsid. Methods of producing rAAV particles are known in the art and are commercially available (see, e.g., Zolotukhin el al. Production and purification of serotype 1, 2, and 5 recombinant adeno-associated viral vectors. Methods 28 (2002) 158-167; and U.S. Patent Application Publication Numbers US 2007/0015238 and US 2012/0322861, which are incorporated herein by reference; and plasmids and kits available from ATCC and Cell Biolabs, Inc.). For example, a plasmid containing the rAAV vector may be combined with one or more helper plasmids, e.g., that contain a rep gene (e.g., encoding Rep78, Rep68, Rep52 and Rep40) and a cap gene (encoding VP1, VP2, and VP3, including a modified VP3 region as described herein), and transfected into a producer cell line such that the rAAV particle can be packaged and subsequently purified.

The rAAV particles or particles within an rAAV preparation disclosed herein, may be of any AAV serotype, including any derivative or pseudotype (e.g., 1, 2, 3, 4, 5, 6, 7, 8, 9, 10, 2/1, 2/5, 2/8, 2/9, 3/1, 3/5, 3/8, or 3/9). As used herein, the serotype of an rAAV an rAAV particle refers to the scrotype of the capsid proteins of the recombinant virus. In some embodiments, the rAAV particle is rAAV6 or rAAV9. In some embodiments, the rAAV particle is AAVrh74. In a preferred embodiment, the rAAV particle is AAVrh74. In an additional preferred embodiment, the rAAV is AAV9. In several embodiments, an rh74 AAV is mutated to advantageously enhance delivery to cardiac tissue, for example by a tryptophan to arginine mutation at amino acid 505 of VP1 capsid, and/or other mutations, as described in PCT Publication WO 2019/178412, which is incorporated in its entirety by reference herein. Non-limiting examples of derivatives, pseudotypes, and/or other vector types include, but are not limited to, AAVrh10, AAVrh74, AAV2/1, AAV2/5, AAV2/6, AAV2/8, AAV2/9, AAV2-AAV3 hybrid, AAVhu.14, AAV3a/3b, AAVrh32.33, AAV-SC15, AAV-HSC17, AAVhu.37, AAVrh8, CHt-P6, AAV2.5, AAV6.2, AAV218, AAV-HSC15/17, AAVM41, AAV9.45, AAV6 (Y445F/Y731F), AAV2.5T, AAV-HAE1/2, AAV clone 32/83, AAVShHIO, AAV2 (Y->F), AAV8 (Y733F), AAV2.15, AAV2.4, AAVM41, and AA Vr3.45.

Such AAV serotypes and derivatives/pseudotypes, and methods of producing such derivatives/pseudotypes are known in the art (see, e.g., Mol Ther. 2012 April;20 (4): 699-708. doi: 10.1038/mt.2011.287. Epub 2012 Jan. 24. The AAV vector toolkit: poised at the clinical crossroads. Asokan Al, Schaffer DV, Samulski RJ.). In particular embodiments, the capsid of any of the herein disclosed rAAV particles is of the AA Vrh10 serotype. In a preferred embodiment, the capsid of the rAAV particle is AAVrh10 serotype. In some embodiments, the capsid is of the AAV2/6 serotype. In some embodiments, the rAAV particle is a pseudotyped rAAV particle, which comprises (a) an rAAV vector comprising ITRs from one serotype (e.g., AAV2, AAV3) and (b) a capsid comprised of capsid proteins derived from another serotype (e.g., AAV1, AAV2, AAV3, AAV4, AAV5, AAV6, AAV7, AAV8, AAV9, or AAV10). Methods for producing and using pseudotyped rAAV vectors are known in the art (see, e.g., Duan et al, J. Virol., 75:7662-7671, 2001; Halbert et al, J. Virol., 74:1524-1532, 2000; Zolotukhin et al, Methods, 28:158-167, 2002; and Auricchio et al., Hum. Molec. Genet., 10:3075-3081, 2001). rAAV Gene Therapy for Heart Diseases.

In some embodiments, the rAAV vectors of the present disclosure further comprise a polyadenylation (pA) signal. For example, in preferred embodiments the pA signal comprises one or both of the following sequences: SEQ ID NOs: 6 and 20.

In some embodiments, the rAAV vectors of the present disclosure comprise at least, in order from 5′ to 3′, a first adeno-associated vims (AAV) inverted terminal repeat (ITR) sequence, a promoter operably linked to a transgene, a polyadenylation signal, and a second AAV inverted terminal repeat (ITR) sequence.

In some embodiments, the rAAV vector genome is circular. In some embodiments, the rAAV vector genome is linear. In some embodiments, the rAAV vector genome is single-stranded. In some embodiments, the rAAV vector genome is double-stranded. In some embodiments, the rAAV genome vector is a self-complementary rAAV vector.

Described herein are non-limiting examples of rAAV vectors. The vectors illustrated below comprise the linearized plasmid sequences set forth as SEQ ID NOs: 1-7 or SEQ ID NOs: 13-21 arranged in sequence. Accordingly, in some embodiments, the rAAV vector may have a sequence having identity to SEQ ID NOs: 1-7 or SEQ ID NOs: 13-21, when those groupings of sequences are arranged in sequence. As used herein, “arranged in sequence” refers to the placement in a vector, in 5′ to 3′ order, of the subject sequences in the grouping. That is, an rAAV vector that has a sequence comprising SEQ ID NOs: 1-7, arranged in sequence, contains, in 5′ to 3′ order, SEQ ID NOs: 1, 2, 3, 4, 5, 6, and 7. The rAAV vectors of the disclosure may comprise nucleotide sequences that have at least 70% identity, at least about 80% identity, at least about 90% identity, at least about 95% identity, at least about 96% identity, at least about 97% identity, at least about 98% identity, at least about 99% identity, at least about 99.5% identity, or at least about 99.9% identity to the sequences set forth as SEQ ID NOs: 1-7 or SEQ ID NOs: 13-21, arranged in sequence. In several embodiments, the rAAV vector has 100% identity to the sequences set forth as SEQ ID NOs 1-7 or ID NOs: 13-21 arranged in sequence. In some embodiments, any of the disclosed rAAV vectors have at least 85% sequence identity to any of the disclosed sequence groupings arranged in sequence, without any gaps between the subject sequences. In some embodiments, any of the disclosed rAAV vectors have at least 85% sequence identity to any of the disclosed sequence groupings arranged in sequence, without any gaps between the subject sequences.

In some embodiments, any of the disclosed rAAV nucleic acid vector sequences comprise truncations at the 5′ or 3′ end relative to the sequences of any one of SEQ ID NOs: 1-7 or 13-21 arranged in sequence. In some embodiments, any of the rAAV vectors comprise a nucleotide sequence that differs from the sequence of any one of SEQ ID NOs: 1-7 or 13-21 arranged in sequence by 1, 2, 3, 4, 5, 6, 7, 8, 9, 10, 11, 12, 13, 14, 15, 16, 17, 18, or more than 18 nucleotides.

In some embodiments, the therapeutic rAAV vector has a sequence comprising SEQ ID NOs: 1-7, arranged in sequence. In some embodiments, the therapeutic rAAV vector has a sequence comprising SEQ ID NOs: 13-21, arranged in sequence.

### Recombinant Adeno-Associated Virus Vectors and Therapeutic Uses Thereof

Many serotypes of AAV have been cloned and sequenced. Serotypes 1 and 6 share >99% amino acid homology in their capsid proteins. Of the first six AAV serotypes, serotype 2 is widely characterized and therefore often used in gene transfer studies, however according to embodiments disclosed herein, other AAV serotypes are also used, such as AAV9, AAV20, AAVrh74, AAVrh10, and the like. In several embodiments, repeat administration of a given serotype that would be expected to elicit a humoral immune response is performed in connection with an immune management regimen. In several embodiments, an immune management regimen comprises administration of one or more agents that function as B-cell depletors, alone, or in conjunction with one or more agents that inhibit one or more aspects of the mTOR pathway. In one embodiment, an antiCD20 antibody is administered and rapamycin is administered. In several embodiments, this allows for the repeat administration of a given serotype rAAV with reduced, limited or no immune response to a subsequent dosing of the rAAV. Further information about immune management can found in United States Patent Publication No. US 2017/0049887, published Feb. 23, 2017, the entire contents of which is incorporated by reference herein.

The therapeutic rAAV vectors, therapeutic rAAV particles, or the composition comprising the therapeutic rAAV particles of the present disclosure, may be used for gene therapy for heart diseases in a human subject in need thereof, such as cardiomyopathies as provided for herein). Examples of heart disease that may be treated using the methods and compositions of the present disclosure include, but are not limited to, cardiomyopathy and acute ischemia. In some embodiments, cardiomyopathy is hypertrophic cardiomyopathy or dilated cardiomyopathy. In some embodiments, the cardiomyopathy is dilated cardiomyopathy and is caused by or associated with reduced or non-existent expression and/or function of RBM20. The therapeutic rAAV vectors, particles, and compositions comprising the therapeutic rAAV particles may be used for treatment of such heart failure (e.g., heart failure secondary to cardiomyopathy) when administered to a subject in need thereof, e.g., via vascular delivery into the coronary arteries and/or direct injection to the heart. The therapeutic rAAV vectors, particles, and compositions comprising the rAAV particles drive the concurrent expression of RBM20 in the cardiomyocytes of the subject.

The amino acid sequence of the therapeutic RBM20 encoded by the RBM20 transgene is at least about 85%, at least 90%, at least 95%, at least 96%, at least 97%, at least 98% or at least 99% sequence identity to the amino acid sequence set forth as SEQ ID NO: 8.

In some embodiments, there are provided amino acid sequences that correspond to any of the nucleic acids disclosed herein (and/or included in the accompanying sequence listing), while accounting for degeneracy of the nucleic acid code. Furthermore, those sequences (whether nucleic acid or amino acid) that vary from those expressly disclosed herein (and/or included in the accompanying sequence listing), but have functional similarity or equivalency are also contemplated within the scope of the present disclosure. The foregoing includes mutants, truncations, substitutions, or other types of modifications.

In accordance with some embodiments described herein, any of the sequences may be used, or a truncated or mutated form of any of the sequences disclosed herein (and/or included in the accompanying sequence listing) may be used and in any combination.

The promoter driving expression of the therapeutic nucleic acid can be, but is not limited to, a constitutive promoter, an inducible promoter, a tissue-specific promoter, a neuronal-specific promoter, a muscle-specific promoter, or a synthetic promoter. In some embodiments, the promoter is a neuronal-specific promoter or a muscle-specific promoter. A constitutive promoter can be, but is not limited to, a Herpes Simplex virus (HSV) promoter, a thymidine kinase (TK) promoter, a Rous Sarcoma Virus (RSV) promoter, a Simian Virus 40 (SV40) promoter, a Mousc Mammary Tumor Virus (MMTV) promoter, an Adenovirus E1A promoter, a cytomegalovirus (CMV) promoter, a mammalian housekeeping gene promoter, or a β-actin promoter. An inducible promoter can be, but is not limited to, a cytochrome P450 gene promoter, a heat shock protein gene promoter, a metallothionein gene promoter, a hormone-inducible gene promoter, an estrogen gene promoter, or a tetVP16 promoter that is responsive to tetracycline. A muscle-specific promoter can be, but is not limited to, desmin promoter, a creatine kinase promoter (e.g., MHCK9), a myogenin promoter, an alpha myosin heavy chain promoter, or a natriuretic peptide promoter.

In some embodiments, the therapeutic rAAV promoter comprises a neuron-specific or cardiac muscle-specific promoter.

The therapeutic rAAV can be serotype 1, serotype 2, serotype 3, serotype 4, serotype 5, serotype 6, serotype 7, serotype 8, serotype 9, serotype 10, serotype 11, serotype 12, serotype rh10, or serotype rh74. The therapeutic rAAV can also be a pseudotyped rAAV.

In some embodiments, the therapeutic rAAV has a sequence sharing at least 85% sequence identity to SEQ ID NOs: 1-7 or ID NOs: 13-21, arranged in sequence.

In some embodiments, the therapeutic rAAV has a sequence sharing at least 95% sequence identity to SEQ ID NOs: 1-7 or ID NOs: 13-21, arranged in sequence.

### In Silico Derivation of Consensus Kozak Sequence for Enhanced Expression in Cardiac Tissues

An analysis of highly expressed genes in human heart tissues was performed to design a novel synthetic Kozak sequence to enhance transgene expression in the heart. Genes were selected from the Human Protein Atlas and Kozak sequences for each were identified in NCBI, as show in Table 1 below. A consensus sequence was derived using Weblogo (https://weblogo.berkeley.edu/logo.cgi). The consensus sequence (AGCCCCAAC (SEQ ID NO: 36)) was then utilized in the design of selected transgene constructs provided herein.

TABLE 1

| Gene | Kozak sequence | SEQ ID: |
|---|---|---|
| MYH7 | GGCACAGCC | 22 |
| ACTC1 | TGTGCCAAG | 23 |
| INNI3 | AGTCTCAGC | 24 |
| MYL7 | GCAGAGAGA | 25 |
| NPPA | TCCAGAGAC | 26 |
| NPPB | TCCAGAGAC | 27 |
| TNNI2 | GACCTCAGG | 28 |
| MYBPC3 | TCTCTCAGG | 29 |
| MYL4 | CAAGACAAC | 30 |
| MYBPHL | AGGCCCAGC | 31 |
| MYH6 | AGCACCAAG | 32 |
| LRRC10 | AGCCTCCGC | 33 |
| ACTC1 | TGTGCCAAG | 34 |
| RD3L | AGGCTAAAA | 35 |
| Consensus Sequence | AGCCCCAAC | 36 |

Self-complementary AAV (scAAV) genomes were designed with various promoters and alternative Kozak sequences, including the in silico derived sequence, as shown below.

In silico Construct 1 IS1. scAAV with chick beta actin (CBA) promoter and AGCGCCACC (SEQ ID NO: 37) Kozak sequence:
Lower case=5′ ITR
Underlined, uppercase=CBA promoter
Upper case, bold=Kozak sequence
Upper case=RBM20 sequence
Upper case, bold underlined=PolyA
Lower case=3′ WT ITR

SEQ ID NO: 38
ttggccactccctctctgcgcgctcgctcgctcactgagg
ccgggcgaccaaaggtcgcccgacgcccgggctttgcccg
ggcggcctcagtgagcgagcgagcgcgcagagagggagtg
gccaactccatcactaggggttcctTCGAGGTGAGCCCCA
CGTTCTGCTTCACTCTCCCCATCTCCCCCCCCTCCCCACC
CCCAATTTTGTATTTATTTATTTTTTAATTATTTTGTGCA
GCGATGGGGGCGGGGGGGGGGGGGGGGCGCGCGCCAGGCG
GGGCGGGGCGGGGCGAGGGGCGGGGGGGGCGAGGCGGAGA
GGTGCGGCGGCAGCCAATCAGAGCGGCGCGCTCCGAAAGT
TTCCTTTTATGGCGAGGCGGCGGCGGCGGCGGCCCTATAA
AAAGCGAAGCGCGCGGCGGGCGAGCGCCACC
ATGGTGCTG
GCAGCAGCCATGAGCCAGGACGCGGACCCCAGCGGTCCGG
AGCAGCCGGACAGAGTTGCCTGCAGTGTGCCTGGTGCCCG
GGCGTCCCCGGCACCCTCCGGCCCGCGAGGGATGCAGCAG
CCGCCGCCGCCGCCCCAGCCACCGCCCCCGCCCCAAGCCG
GCCTACCCCAGATCATCCAAAATGCCGCCAAGCTCCTGGA
CAAGAACCCATTCTCGGTCAGTAACCCGAACCCTCTGCTT
CCTTCACCTGCCAGTCTCCAGCTGGCTCAACTGCAGGCCC
AGCTCACCCTCCACCGGCTGAAGCTGGCACAGACAGCTGT
CACCAACAACACTGCAGCCGCCACAGTCCTGAACCAAGTC
CTCTCCAAAGTGGCCATGTCCCAGCCTCTCTTCAATCAAC
TGAGGCATCCGTCTGTGATCACTGGCCCCCACGGCCATGC
TGGGGTTCCCCAACATGCTGCAGCCATACCCAGTACCCGG
TTTCCCTCTAATGCAATTGCCTTTTCACCCCCCAGCCAGA
CACGAGGCCCCGGACCCTCCATGAACCTTCCCAACCAGCC
ACCCAGTGCCATGGTGATGCATCCTTTCACTGGGGTAATG
CCTCAGACCCCTGGCCAGCCAGCAGTCATCTTGGGCATTG
GCAAGACTGGGCCTGCTCCAGCTACAGCAGGATTCTATGA
GTATGGCAAAGCCAGCTCTGGCCAGACATATGGCCCTGAA
ACAGATGGTCAGCCTGGCTTCCTGCCATCCTCGGCCTCAA
CCTCGGGCAGTGTGACCTATGAAGGGCACTACAGCCACAC
AGGGCAGGATGGTCAAGCTGCCTTTTCCAAAGATTTTTAC
GGACCCAACTCCCAAGGTTCACATGTGGCCAGCGGATTTC
CAGCTGAGCAGGCTGGGGGCCTGAAAAGTGAGGTCGGGCC
ACTGCTGCAGGGCACAAACAGCCAATGGGAGAGCCCCCAT
GGATTCTCGGGCCAAAGCAAGCCTGATCTCACAGCAGGTC
CCATGTGGCCTCCACCCCACAACCAGCCCTATGAGCTGTA
CGACCCCGAGGAACCAACCTCAGACAGGACACCTCCTTCC
TTCGGGGGTCGGCTTAACAACAGCAAACAGGGTTTTATCG
GTGCTGGGCGGAGGGCCAAGGAGGACCAGGCGTTGCTATC
TGTGCGGCCTCTGCAGGCTCATGAGCTGAACGACTTTCAC
GGTGTGGCCCCCCTCCACTTGCCGCATATCTGTAGCATCT
GTGACAAGAAGGTGTTTGATTTGAAGGACTGGGAGCTGCA
TGTGAAAGGGAAGCTGCACGCTCAGAAATGCCTGGTCTTC
TCTGAAAATGCTGGCATCCGGTGTATACTTGGTTCGGCAG
AGGGAACATTGTGTGCTTCTCCCAACAGCACAGCTGTTTA
TAACCCTGCTGGGAATGAAGATTATGCCTCAAATCTTGGA
ACATCATACGTGCCCATTCCAGCAAGGTCATTCACTCAGT
CAAGCCCCACATTTCCTTTGGCTTCTGTGGGGACAACTTT
TGCACAGCGGAAAGGGGCTGGCCGTGTGGTGCACATCTGC
AATCTCCCTGAAGGAAGCTGCACTGAGAATGACGTCATTA
ACCTGGGGCTGCCCTTTGGAAAGGTCACTAATTACATCCT
CATGAAATCGACTAATCAGGCCTTTTTAGAGATGGCTTAC
ACAGAAGCTGCACAGGCCATGGTCCAGTATTATCAAGAAA
AATCTGCTGTGATCAATGGTGAGAAGTTGCTCATTCGGAT
GTCCAAGAGATACAAGGAATTGCAGCTCAAGAAACCCGGG
AAGGCCGTGGCTGCCATCATCCAGGACATCCATTCCCAGA
GGGAGAGGGACATGTTCCGGGAAGCAGACAGATATGGCCC
AGAAAGGCCGCGGTCTCGTAGTCCGGTGAGCCGGTCACTC
TCCCCGAGGTCCCACACTCCCAGCTTCACCTCCTGCAGCT
CTTCCCACAGCCCTCCGGGCCCCTCCCGGGCTGACTGGGG
CAATGGCCGGGACTCCTGGGAGCACTCTCCCTATGCCAGG
AGGGAGGAAGAGCGAGACCCGGCTCCCTGGAGGGACAACG
GAGATGACAAGAGGGACAGGATGGACCCCTGGGCACATGA
TCGCAAACACCACCCCCGGCAACTGGACAAGGCTGAGTTG
GACGAGCGACCAGAAGGAGGGAGGCCCCACCGGGAGAAGT
ACCCGAGATCTGGGTCTCCCAACCTGCCCCACTCTGTGTC
CAGCTACAAAAGCCGTGAAGACGGCTACTACCGGAAAGAG
CCCAAAGCCAAGTGGGACAAGTATCTGAAGCAGCAGCAGG
ATGCCCCCGGGAGGTCCAGGAGGAAAGACGAGGCCAGGCT
GCGGGAAAGCAGACACCCCCATCCGGATGACTCAGGCAAG
GAAGATGGGCTGGGGCCAAAGGTCACTAGGGCCCCTGAGG
GCGCCAAGGCCAAGCAGAATGAGAAAAATAAAACCAAGAG
AACTGATAGAGACCAAGAAGGAGCTGATGATAGAAAAGAA
AACACAATGGCAGAGAATGAGGCTGGAAAAGAGGAACAGG
AGGGCATGGAAGAAAGCCCTCAATCAGTGGGCAGACAGGA
GAAAGAAGCAGAGTTCTCTGATCCGGAAAACACAAGGACA
AAGAAGGAACAAGATTGGGAGAGTGAAAGTGAGGCAGAGG
GGGAGAGCTGGTATCCCACTAACATGGAGGAGCTGGTGAC
AGTGGACGAGGTTGGGGAAGAAGAAGATTTTATCGTGGAA
CCAGACATCCCAGAGCTGGAAGAAATTGTGCCCATTGACC
AGAAAGACAAAATTTGCCCAGAAACATGTCTGTGTGTGAC
AACCACCTTAGACTTAGACCTGGCCCAGGATTTCCCCAAG
GAAGGAGTCAAGGCCGTAGGGAATGGGGCTGCAGAAATCA
GCCTCAAGTCACCCAGAGAACTGCCCTCTGCTTCCACAAG
CTGTCCCAGTGACATGGACGTGGAAATGCCTGGCCTAAAT
CTGGATGCTGAGCGGAAGCCAGCTGAAAGTGAGACAGGCC
TCTCCCTGGAGGATTCAGATTGCTACGAGAAGGAGGCAAA
GGGAGTGGAGAGCTCAGATGTTCATCCAGCCCCTACAGTC
CAGCAAATGTCTTCCCCTAAGCCAGCAGAGGAGAGGGCCC
GGCAGCCAAGCCCATTTGTGGATGATTGCAAGACCAGGGG
GACCCCCGAAGATGGGGCTTGTGAAGGCAGCCCCCTGGAG
GAGAAAGCCAGCCCCCCCATCGAAACTGACCTCCAAAACC
AAGCCTGCCAAGAAGTGTTGACCCCGGAAAACTCCAGGTA
CGTGGAAATGAAATCTCTGGAGGTGAGGTCACCAGAGTAC
ACTGAAGTGGAACTGAAACAGCCCCTTTCTTTGCCCTCTT
GGGAACCAGAGGATGTGTTCAGTGAACTTAGCATTCCTCT
AGGGGTGGAGTTCGTGGTTCCCAGGACTGGCTTTTATTGC
AAGCTGTGTGGGCTGTTCTACACGAGCGAGGAGACAGCAA
AGATGAGCCACTGCCGCAGCGCTGTCCACTACAGGAACTT
ACAGAAATATTTGTCCCAGCTGGCCGAGGAGGGCCTCAAG
GAGACCGAGGGGGCAGATAGCCCGAGGCCAGAGGACAGCG
GAATCGTGCCACGCTTCGAAAGGAAAAAGCTCTG
AAATAAAAGATCCTTATTTTCATTG
GATCTGTGTGTTGGTTTTTTGTGTG
aggaacccctagtgatggagttggccactccctctc
tgcgcgctcgctcgctcactgaggccggggaccaaaggtc
gcccgacgcccgggctttgcccgggggcctcagtgagcga
gcgagcgcgcagagagggagtggccaa

In silico Construct 2 IS2. scAAV with chick beta actin (CBA) promoter and in silico derived Kozak sequence:
Lower case=5′ ITR
Underlined, uppercase=CBA promoter
Upper case, bold=in silico derived Kozak sequence
Upper case=RBM20 cDNA
Upper case, bold underlined=PolyA

SEQ ID NO: 39
ttggccactccctctctgcgcgctcgctcgctcactgaggccg
ggcgaccaaaggtcgcccgacgcccgggctttgcccgggcggc
ctcagtgagcgagcgagcgcgcagagagggagtggccaactcc
atcactaggggttcctTCGAGGTGAGCCCCACGTTCTGCTTCA
CTCTCCCCATCTCCCCCCCCTCCCCACCCCCAATTTTGTATTT
ATTTATTTTTTAATTATTTTGTGCAGCGATGGGGGCGGGGGGG
GGGGGGGGGCGCGCGCCAGGCGGGGCGGGGGGGGCGAGGGGGG
GGCGGGGCGAGGCGGAGAGGTGCGGCGGCAGCCAATCAGAGCG
GCGCGCTCCGAAAGTTTCCTTTTATGGCGAGGCGGCGGCGGCG
GCGGCCCTATAAAAAGCGAAGCGCGCGGCGGGCG
AGCCCCAACATGGTGCTGGCAGCAGCCATGAGCC
AGGACGCGGACCCCAGCGGTCCGGAGCAGCCGGACAGAGTTGC
CTGCAGTGTGCCTGGTGCCCGGGCGTCCCCGGCACCCTCCGGC
CCGCGAGGGATGCAGCAGCCGCCGCCGCCGCCCCAGCCACCGC
CCCCGCCCCAAGCCGGCCTACCCCAGATCATCCAAAATGCCGC
CAAGCTCCTGGACAAGAACCCATTCTCGGTCAGTAACCCGAAC
CCTCTGCTTCCTTCACCTGCCAGTCTCCAGCTGGCTCAACTGC
AGGCCCAGCTCACCCTCCACCGGCTGAAGCTGGCACAGACAGC
TGTCACCAACAACACTGCAGCCGCCACAGTCCTGAACCAAGTC
CTCTCCAAAGTGGCCATGTCCCAGCCTCTCTTCAATCAACTGA
GGCATCCGTCTGTGATCACTGGCCCCCACGGCCATGCTGGGGT
TCCCCAACATGCTGCAGCCATACCCAGTACCCGGTTTCCCTCT
AATGCAATTGCCTTTTCACCCCCCAGCCAGACACGAGGCCCCG
GACCCTCCATGAACCTTCCCAACCAGCCACCCAGTGCCATGGT
GATGCATCCTTTCACTGGGGTAATGCCTCAGACCCCTGGCCAG
CCAGCAGTCATCTTGGGCATTGGCAAGACTGGGCCTGCTCCAG
CTACAGCAGGATTCTATGAGTATGGCAAAGCCAGCTCTGGCCA
GACATATGGCCCTGAAACAGATGGTCAGCCTGGCTTCCTGCCA
TCCTCGGCCTCAACCTCGGGCAGTGTGACCTATGAAGGGCACT
ACAGCCACACAGGGCAGGATGGTCAAGCTGCCTTTTCCAAAGA
TTTTTACGGACCCAACTCCCAAGGTTCACATGTGGCCAGCGGA
TTTCCAGCTGAGCAGGCTGGGGGCCTGAAAAGTGAGGTCGGGC
CACTGCTGCAGGGCACAAACAGCCAATGGGAGAGCCCCCATGG
ATTCTCGGGCCAAAGCAAGCCTGATCTCACAGCAGGTCCCATG
TGGCCTCCACCCCACAACCAGCCCTATGAGCTGTACGACCCCG
AGGAACCAACCTCAGACAGGACACCTCCTTCCTTCGGGGGTCG
GCTTAACAACAGCAAACAGGGTTTTATCGGTGCTGGGCGGAGG
GCCAAGGAGGACCAGGCGTTGCTATCTGTGCGGCCTCTGCAGG
CTCATGAGCTGAACGACTTTCACGGTGTGGCCCCCCTCCACTT
GCCGCATATCTGTAGCATCTGTGACAAGAAGGTGTTTGATTTG
AAGGACTGGGAGCTGCATGTGAAAGGGAAGCTGCACGCTCAGA
AATGCCTGGTCTTCTCTGAAAATGCTGGCATCCGGTGTATACT
TGGTTCGGCAGAGGGAACATTGTGTGCTTCTCCCAACAGCACA
GCTGTTTATAACCCTGCTGGGAATGAAGATTATGCCTCAAATC
TTGGAACATCATACGTGCCCATTCCAGCAAGGTCATTCACTCA
GTCAAGCCCCACATTTCCTTTGGCTTCTGTGGGGACAACTTTT
GCACAGCGGAAAGGGGCTGGCCGTGTGGTGCACATCTGCAATC
TCCCTGAAGGAAGCTGCACTGAGAATGACGTCATTAACCTGGG
GCTGCCCTTTGGAAAGGTCACTAATTACATCCTCATGAAATCG
ACTAATCAGGCCTTTTTAGAGATGGCTTACACAGAAGCTGCAC
AGGCCATGGTCCAGTATTATCAAGAAAAATCTGCTGTGATCAA
TGGTGAGAAGTTGCTCATTCGGATGTCCAAGAGATACAAGGAA
TTGCAGCTCAAGAAACCCGGGAAGGCCGTGGCTGCCATCATCC
AGGACATCCATTCCCAGAGGGAGAGGGACATGTTCCGGGAAGC
AGACAGATATGGCCCAGAAAGGCCGCGGTCTCGTAGTCCGGTG
AGCCGGTCACTCTCCCCGAGGTCCCACACTCCCAGCTTCACCT
CCTGCAGCTCTTCCCACAGCCCTCCGGGCCCCTCCCGGGCTGA
CTGGGGCAATGGCCGGGACTCCTGGGAGCACTCTCCCTATGCC
AGGAGGGAGGAAGAGCGAGACCCGGCTCCCTGGAGGGACAACG
GAGATGACAAGAGGGACAGGATGGACCCCTGGGCACATGATCG
CAAACACCACCCCCGGCAACTGGACAAGGCTGAGTTGGACGAG
CGACCAGAAGGAGGGAGGCCCCACCGGGAGAAGTACCCGAGAT
CTGGGTCTCCCAACCTGCCCCACTCTGTGTCCAGCTACAAAAG
CCGTGAAGACGGCTACTACCGGAAAGAGCCCAAAGCCAAGTGG
GACAAGTATCTGAAGCAGCAGCAGGATGCCCCCGGGAGGTCCA
GGAGGAAAGACGAGGCCAGGCTGCGGGAAAGCAGACACCCCCA
TCCGGATGACTCAGGCAAGGAAGATGGGCTGGGGCCAAAGGTC
ACTAGGGCCCCTGAGGGCGCCAAGGCCAAGCAGAATGAGAAAA
ATAAAACCAAGAGAACTGATAGAGACCAAGAAGGAGCTGATGA
TAGAAAAGAAAACACAATGGCAGAGAATGAGGCTGGAAAAGAG
GAACAGGAGGGCATGGAAGAAAGCCCTCAATCAGTGGGCAGAC
AGGAGAAAGAAGCAGAGTTCTCTGATCCGGAAAACACAAGGAC
AAAGAAGGAACAAGATTGGGAGAGTGAAAGTGAGGCAGAGGGG
GAGAGCTGGTATCCCACTAACATGGAGGAGCTGGTGACAGTGG
ACGAGGTTGGGGAAGAAGAAGATTTTATCGTGGAACCAGACAT
CCCAGAGCTGGAAGAAATTGTGCCCATTGACCAGAAAGACAAA
ATTTGCCCAGAAACATGTCTGTGTGTGACAACCACCTTAGACT
TAGACCTGGCCCAGGATTTCCCCAAGGAAGGAGTCAAGGCCGT
AGGGAATGGGGCTGCAGAAATCAGCCTCAAGTCACCCAGAGAA
CTGCCCTCTGCTTCCACAAGCTGTCCCAGTGACATGGACGTGG
AAATGCCTGGCCTAAATCTGGATGCTGAGCGGAAGCCAGCTGA
AAGTGAGACAGGCCTCTCCCTGGAGGATTCAGATTGCTACGAG
AAGGAGGCAAAGGGAGTGGAGAGCTCAGATGTTCATCCAGCCC
CTACAGTCCAGCAAATGTCTTCCCCTAAGCCAGCAGAGGAGAG
GGCCCGGCAGCCAAGCCCATTTGTGGATGATTGCAAGACCAGG
GGGACCCCCGAAGATGGGGCTTGTGAAGGCAGCCCCCTGGAGG
AGAAAGCCAGCCCCCCCATCGAAACTGACCTCCAAAACCAAGC
CTGCCAAGAAGTGTTGACCCCGGAAAACTCCAGGTACGTGGAA
ATGAAATCTCTGGAGGTGAGGTCACCAGAGTACACTGAAGTGG
AACTGAAACAGCCCCTTTCTTTGCCCTCTTGGGAACCAGAGGA
TGTGTTCAGTGAACTTAGCATTCCTCTAGGGGTGGAGTTCGTG
GTTCCCAGGACTGGCTTTTATTGCAAGCTGTGTGGGCTGTTCT
ACACGAGCGAGGAGACAGCAAAGATGAGCCACTGCCGCAGCGC
TGTCCACTACAGGAACTTACAGAAATATTTGTCCCAGCTGGCC
GAGGAGGGCCTCAAGGAGACCGAGGGGGCAGATAGCCCGAGGC
CAGAGGACAGCGGAATCGTGCCACGCTTCGAAAGGAAAAAGCT
CTG
AAATAAAAGATCCTTATTTTCATTG
GATCTGTGTGTTGGTTTTTTGTGTG
aggaacccctagtgatggagttggccactccctctctgcgcgc
tcgctcgctcactgaggccgggcgaccaaaggtcgcccgacgc
ccgggctttgcccgggcggcctcagtgagcgagcgagcgcgca
gagagggagtggccaa

In silico Construct 3 IS3. scAAV with chick beta actin (CBA) promoter and CAACCCAGC Kozak sequence:
Lower case=5′ ITR
Underlined, uppercase=CBA promoter
Upper case, bold=Kozak sequence
Upper case=RBM20 sequence
Upper case, bold underlined=PolyA

SEQ ID NO: 40
ttggccactccctctctgcgcgctcgctcgctcactgaggccg
ggcgaccaaaggtcgcccgacgcccgggctttgcccgggggcc
tcagtgagcgagcgagcgcgcagagagggagtggccaactcca
tcactaggggttcctTCGAGGTGAGCCCCACGTTCTGCTTCAC
TCTCCCCATCTCCCCCCCCTCCCCACCCCCAATTTTGTATTTA
TTTATTTTTTAATTATTTTGTGCAGCGATGGGGGCGGGGGGGG
GGGGGGGGCGCGCGCCAGGCGGGGCGGGGCGGGGCGAGGGGCG
GGGCGGGGCGAGGCGGAGAGGTGCGGCGGCAGCCAATCAGAGC
GGCGCGCTCCGAAAGTTTCCTTTTATGGCGAGGCGGCGGCGGC
GGCGGCCCTATAAAAAGCGAAGCGCGCGGCGGGCG
CAACCCAGCATGGTGCTGGCAGCAGCCATGAGCCA
GGACGCGGACCCCAGCGGTCCGGAGCAGCCGGACAGAGTTGCC
TGCAGTGTGCCTGGTGCCCGGGCGTCCCCGGCACCCTCCGGCC
CGCGAGGGATGCAGCAGCCGCCGCCGCCGCCCCAGCCACCGCC
CCCGCCCCAAGCCGGCCTACCCCAGATCATCCAAAATGCCGCC
AAGCTCCTGGACAAGAACCCATTCTCGGTCAGTAACCCGAACC
CTCTGCTTCCTTCACCTGCCAGTCTCCAGCTGGCTCAACTGCA
GGCCCAGCTCACCCTCCACCGGCTGAAGCTGGCACAGACAGCT
GTCACCAACAACACTGCAGCCGCCACAGTCCTGAACCAAGTCC
TCTCCAAAGTGGCCATGTCCCAGCCTCTCTTCAATCAACTGAG
GCATCCGTCTGTGATCACTGGCCCCCACGGCCATGCTGGGGTT
CCCCAACATGCTGCAGCCATACCCAGTACCCGGTTTCCCTCTA
ATGCAATTGCCTTTTCACCCCCCAGCCAGACACGAGGCCCCGG
ACCCTCCATGAACCTTCCCAACCAGCCACCCAGTGCCATGGTG
ATGCATCCTTTCACTGGGGTAATGCCTCAGACCCCTGGCCAGC
CAGCAGTCATCTTGGGCATTGGCAAGACTGGGCCTGCTCCAGC
TACAGCAGGATTCTATGAGTATGGCAAAGCCAGCTCTGGCCAG
ACATATGGCCCTGAAACAGATGGTCAGCCTGGCTTCCTGCCAT
CCTCGGCCTCAACCTCGGGCAGTGTGACCTATGAAGGGCACTA
CAGCCACACAGGGCAGGATGGTCAAGCTGCCTTTTCCAAAGAT
TTTTACGGACCCAACTCCCAAGGTTCACATGTGGCCAGCGGAT
TTCCAGCTGAGCAGGCTGGGGGCCTGAAAAGTGAGGTCGGGCC
ACTGCTGCAGGGCACAAACAGCCAATGGGAGAGCCCCCATGGA
TTCTCGGGCCAAAGCAAGCCTGATCTCACAGCAGGTCCCATGT
GGCCTCCACCCCACAACCAGCCCTATGAGCTGTACGACCCCGA
GGAACCAACCTCAGACAGGACACCTCCTTCCTTCGGGGGTCGG
CTTAACAACAGCAAACAGGGTTTTATCGGTGCTGGGCGGAGGG
CCAAGGAGGACCAGGCGTTGCTATCTGTGCGGCCTCTGCAGGC
TCATGAGCTGAACGACTTTCACGGTGTGGCCCCCCTCCACTTG
CCGCATATCTGTAGCATCTGTGACAAGAAGGTGTTTGATTTGA
AGGACTGGGAGCTGCATGTGAAAGGGAAGCTGCACGCTCAGAA
ATGCCTGGTCTTCTCTGAAAATGCTGGCATCCGGTGTATACTT
GGTTCGGCAGAGGGAACATTGTGTGCTTCTCCCAACAGCACAG
CTGTTTATAACCCTGCTGGGAATGAAGATTATGCCTCAAATCT
TGGAACATCATACGTGCCCATTCCAGCAAGGTCATTCACTCAG
TCAAGCCCCACATTTCCTTTGGCTTCTGTGGGGACAACTTTTG
CACAGCGGAAAGGGGCTGGCCGTGTGGTGCACATCTGCAATCT
CCCTGAAGGAAGCTGCACTGAGAATGACGTCATTAACCTGGGG
CTGCCCTTTGGAAAGGTCACTAATTACATCCTCATGAAATCGA
CTAATCAGGCCTTTTTAGAGATGGCTTACACAGAAGCTGCACA
GGCCATGGTCCAGTATTATCAAGAAAAATCTGCTGTGATCAAT
GGTGAGAAGTTGCTCATTCGGATGTCCAAGAGATACAAGGAAT
TGCAGCTCAAGAAACCCGGGAAGGCCGTGGCTGCCATCATCCA
GGACATCCATTCCCAGAGGGAGAGGGACATGTTCCGGGAAGCA
GACAGATATGGCCCAGAAAGGCCGCGGTCTCGTAGTCCGGTGA
GCCGGTCACTCTCCCCGAGGTCCCACACTCCCAGCTTCACCTC
CTGCAGCTCTTCCCACAGCCCTCCGGGCCCCTCCCGGGCTGAC
TGGGGCAATGGCCGGGACTCCTGGGAGCACTCTCCCTATGCCA
GGAGGGAGGAAGAGCGAGACCCGGCTCCCTGGAGGGACAACGG
AGATGACAAGAGGGACAGGATGGACCCCTGGGCACATGATCGC
AAACACCACCCCCGGCAACTGGACAAGGCTGAGTTGGACGAGC
GACCAGAAGGAGGGAGGCCCCACCGGGAGAAGTACCCGAGATC
TGGGTCTCCCAACCTGCCCCACTCTGTGTCCAGCTACAAAAGC
CGTGAAGACGGCTACTACCGGAAAGAGCCCAAAGCCAAGTGGG
ACAAGTATCTGAAGCAGCAGCAGGATGCCCCCGGGAGGTCCAG
GAGGAAAGACGAGGCCAGGCTGCGGGAAAGCAGACACCCCCAT
CCGGATGACTCAGGCAAGGAAGATGGGCTGGGGCCAAAGGTCA
CTAGGGCCCCTGAGGGCGCCAAGGCCAAGCAGAATGAGAAAAA
TAAAACCAAGAGAACTGATAGAGACCAAGAAGGAGCTGATGAT
AGAAAAGAAAACACAATGGCAGAGAATGAGGCTGGAAAAGAGG
AACAGGAGGGCATGGAAGAAAGCCCTCAATCAGTGGGCAGACA
GGAGAAAGAAGCAGAGTTCTCTGATCCGGAAAACACAAGGACA
AAGAAGGAACAAGATTGGGAGAGTGAAAGTGAGGCAGAGGGGG
AGAGCTGGTATCCCACTAACATGGAGGAGCTGGTGACAGTGGA
CGAGGTTGGGGAAGAAGAAGATTTTATCGTGGAACCAGACATC
CCAGAGCTGGAAGAAATTGTGCCCATTGACCAGAAAGACAAAA
TTTGCCCAGAAACATGTCTGTGTGTGACAACCACCTTAGACTT
AGACCTGGCCCAGGATTTCCCCAAGGAAGGAGTCAAGGCCGTA
GGGAATGGGGCTGCAGAAATCAGCCTCAAGTCACCCAGAGAAC
TGCCCTCTGCTTCCACAAGCTGTCCCAGTGACATGGACGTGGA
AATGCCTGGCCTAAATCTGGATGCTGAGCGGAAGCCAGCTGAA
AGTGAGACAGGCCTCTCCCTGGAGGATTCAGATTGCTACGAGA
AGGAGGCAAAGGGAGTGGAGAGCTCAGATGTTCATCCAGCCCC
TACAGTCCAGCAAATGTCTTCCCCTAAGCCAGCAGAGGAGAGG
GCCCGGCAGCCAAGCCCATTTGTGGATGATTGCAAGACCAGGG
GGACCCCCGAAGATGGGGCTTGTGAAGGCAGCCCCCTGGAGGA
GAAAGCCAGCCCCCCCATCGAAACTGACCTCCAAAACCAAGCC
TGCCAAGAAGTGTTGACCCCGGAAAACTCCAGGTACGTGGAAA
TGAAATCTCTGGAGGTGAGGTCACCAGAGTACACTGAAGTGGA
ACTGAAACAGCCCCTTTCTTTGCCCTCTTGGGAACCAGAGGAT
GTGTTCAGTGAACTTAGCATTCCTCTAGGGGTGGAGTTCGTGG
TTCCCAGGACTGGCTTTTATTGCAAGCTGTGTGGGCTGTTCTA
CACGAGCGAGGAGACAGCAAAGATGAGCCACTGCCGCAGCGCT
GTCCACTACAGGAACTTACAGAAATATTTGTCCCAGCTGGCCG
AGGAGGGCCTCAAGGAGACCGAGGGGGCAGATAGCCCGAGGCC
AGAGGACAGCGGAATCGTGCCACGCTTCGAAAGGAAAAAGCTC
TG
AAATAAAAGATCCTTATTTTCATTG
GATCTGTGTGTTGGTTTTTTGTGTG
aggaacccctagtgatggagttggccactccctctctgcgcgc
tcgctcgctcactgaggccgggcgaccaaaggtcgcccgacgc
ccgggctttgcccgggcggcctcagtgagcgagcgagcgcgca
gagagggagtggccaa

In silico Construct 4 IS4. scAAV with muscle creatine kinase (MCK) promoter and AGCGCCACC Kozak sequence:
Lower case=5′ ITR
Underlined, uppercase=MCK promoter
Upper case, bold=Kozak sequence
Upper case=RBM20 sequence
Upper case, bold underlined=PolyA
Lower case=3′ WT ITR

SEQ ID NO: 41
ttggccactccctctctgcgcgctcgctcgctcactgaggccg
ggcgaccaaaggtcgcccgacgcccgggctttgcccgggcggc
ctcagtgagcgagcgagcgcgcagagagggagtggccaactcc
atcactaggggttcctCAAGGCTGTGGGGGACTGAGGGCAGGC
TGTAACAGGCTTGGGGGCCAGGGCTTATACGTGCCTGGGACTC
CCAAAGTATTACTGTTCCATGTTCCCGGCGAAGGGCCAGCTGT
CCCCCGCCAGCTAGACTCAGCACTTAGTTTAGGAACCAGTGAG
CAAGTCAGCCCTTGGGGCAGCCCATACAAGGCCATGGGGCTGG
GCAAGCTGCACGCCTGGGTCCGGGGTGGGCACGGTGCCCGGGC
AACGAGCTGAAAGCTCATCTGCTCTCAGGGGCCCCTCCCTGGG
GACAGCCCCTCCTGGCTAGTCACACCCTGTAGGCTCCTCTATA
TAACCCAGGGGCACAGGGGCTGCCCTC
AGCGCCACCATGGTGCTGGCAGCAGCC
ATGAGCCAGGACGCGGACCCCAGCGGTCCGGAGCAGCCGGACA
GAGTTGCCTGCAGTGTGCCTGGTGCCCGGGCGTCCCCGGCACC
CTCCGGCCCGCGAGGGATGCAGCAGCCGCCGCCGCCGCCCCAG
CCACCGCCCCCGCCCCAAGCCGGCCTACCCCAGATCATCCAAA
ATGCCGCCAAGCTCCTGGACAAGAACCCATTCTCGGTCAGTAA
CCCGAACCCTCTGCTTCCTTCACCTGCCAGTCTCCAGCTGGCT
CAACTGCAGGCCCAGCTCACCCTCCACCGGCTGAAGCTGGCAC
AGACAGCTGTCACCAACAACACTGCAGCCGCCACAGTCCTGAA
CCAAGTCCTCTCCAAAGTGGCCATGTCCCAGCCTCTCTTCAAT
CAACTGAGGCATCCGTCTGTGATCACTGGCCCCCACGGCCATG
CTGGGGTTCCCCAACATGCTGCAGCCATACCCAGTACCCGGTT
TCCCTCTAATGCAATTGCCTTTTCACCCCCCAGCCAGACACGA
GGCCCCGGACCCTCCATGAACCTTCCCAACCAGCCACCCAGTG
CCATGGTGATGCATCCTTTCACTGGGGTAATGCCTCAGACCCC
TGGCCAGCCAGCAGTCATCTTGGGCATTGGCAAGACTGGGCCT
GCTCCAGCTACAGCAGGATTCTATGAGTATGGCAAAGCCAGCT
CTGGCCAGACATATGGCCCTGAAACAGATGGTCAGCCTGGCTT
CCTGCCATCCTCGGCCTCAACCTCGGGCAGTGTGACCTATGAT
AGGGCACTACAGCCACACAGGGCAGGATGGTCAAGCTGCCTTT
TCCAAAGATTTTACGGACCCAACTCCCAAGGTTCACATGTGGC
CAGCGGATTTCCAGCTGAGCAGGCTGGGGGCCTGAAAAGTGAG
GTCGGGCCACTGCTGCAGGGCACAAACAGCCAATGGGAGAGCC
CCCATGGATTCTCGGGCCAAAGCAAGCCTGATCTCACAGCAGG
TCCCATGTGGCCTCCACCCCACAACCAGCCCTATGAGCTGTAC
GACCCCGAGGAACCAACCTCAGACAGGACACCTCCTTCCTTCG
GGGGTCGGCTTAACAACAGCAAACAGGGTTTTATCGGTGCTGG
GCGGAGGGCCAAGGAGGACCAGGCGTTGCTATCTGTGCGGCCT
CTGCAGGCTCATGAGCTGAACGACTTTCACGGTGTGGCCCCCC
TCCACTTGCCGCATATCTGTAGCATCTGTGACAAGAAGGTGTT
TGATTTGAAGGACTGGGAGCTGCATGTGAAAGGGAAGCTGCAC
GCTCAGAAATGCCTGGTCTTCTCTGAAAATGCTGGCATCCGGT
GTATACTTGGTTCGGCAGAGGGAACATTGTGTGCTTCTCCCAA
CAGCACAGCTGTTTATAACCCTGCTGGGAATGAAGATTATGCC
TCAAATCTTGGAACATCATACGTGCCCATTCCAGCAAGGTCAT
TCACTCAGTCAAGCCCCACATTTCCTTTGGCTTCTGTGGGGAC
AACTTTTGCACAGCGGAAAGGGGCTGGCCGTGTGGTGCACATC
TGCAATCTCCCTGAAGGAAGCTGCACTGAGAATGACGTCATTA
ACCTGGGGCTGCCCTTTGGAAAGGTCACTAATTACATCCTCAT
GAAATCGACTAATCAGGCCTTTTTAGAGATGGCTTACACAGAA
GCTGCACAGGCCATGGTCCAGTATTATCAAGAAAAATCTGCTG
TGATCAATGGTGAGAAGTTGCTCATTCGGATGTCCAAGAGATA
CAAGGAATTGCAGCTCAAGAAACCCGGGAAGGCCGTGGCTGCC
ATCATCCAGGACATCCATTCCCAGAGGGAGAGGGACATGTTCC
GGGAAGCAGACAGATATGGCCCAGAAAGGCCGCGGTCTCGTAG
TCCGGTGAGCCGGTCACTCTCCCCGAGGTCCCACACTCCCAGC
TTCACCTCCTGCAGCTCTTCCCACAGCCCTCCGGGCCCCTCCC
GGGCTGACTGGGGCAATGGCCGGGACTCCTGGGAGCACTCTCC
CTATGCCAGGAGGGAGGAAGAGCGAGACCCGGCTCCCTGGAGG
GACAACGGAGATGACAAGAGGGACAGGATGGACCCCTGGGCAC
ATGATCGCAAACACCACCCCCGGCAACTGGACAAGGCTGAGTT
GGACGAGCGACCAGAAGGAGGGAGGCCCCACCGGGAGAAGTAC
CCGAGATCTGGGTCTCCCAACCTGCCCCACTCTGTGTCCAGCT
ACAAAAGCCGTGAAGACGGCTACTACCGGAAAGAGCCCAAAGC
CAAGTGGGACAAGTATCTGAAGCAGCAGCAGGATGCCCCCGGG
AGGTCCAGGAGGAAAGACGAGGCCAGGCTGCGGGAAAGCAGAC
ACCCCCATCCGGATGACTCAGGCAAGGAAGATGGGCTGGGGCC
AAAGGTCACTAGGGCCCCTGAGGGCGCCAAGGCCAAGCAGAAT
GAGAAAAATAAAACCAAGAGAACTGATAGAGACCAAGAAGGAG
CTGATGATAGAAAAGAAAACACAATGGCAGAGAATGAGGCTGG
AAAAGAGGAACAGGAGGGCATGGAAGAAAGCCCTCAATCAGTG
GGCAGACAGGAGAAAGAAGCAGAGTTCTCTGATCCGGAAAACA
CAAGGACAAAGAAGGAACAAGATTGGGAGAGTGAAAGTGAGGC
AGAGGGGGAGAGCTGGTATCCCACTAACATGGAGGAGCTGGTG
ACAGTGGACGAGGTTGGGGAAGAAGAAGATTTTATCGTGGAAC
CAGACATCCCAGAGCTGGAAGAAATTGTGCCCATTGACCAGAA
AGACAAAATTTGCCCAGAAACATGTCTGTGTGTGACAACCACC
TTAGACTTAGACCTGGCCCAGGATTTCCCCAAGGAAGGAGTCA
AGGCCGTAGGGAATGGGGCTGCAGAAATCAGCCTCAAGTCACC
CAGAGAACTGCCCTCTGCTTCCACAAGCTGTCCCAGTGACATG
GACGTGGAAATGCCTGGCCTAAATCTGGATGCTGAGCGGAAGC
CAGCTGAAAGTGAGACAGGCCTCTCCCTGGAGGATTCAGATTG
CTACGAGAAGGAGGCAAAGGGAGTGGAGAGCTCAGATGTTCAT
CCAGCCCCTACAGTCCAGCAAATGTCTTCCCCTAAGCCAGCAG
AGGAGAGGGCCCGGCAGCCAAGCCCATTTGTGGATGATTGCAA
GACCAGGGGGACCCCCGAAGATGGGGCTTGTGAAGGCAGCCCC
CTGGAGGAGAAAGCCAGCCCCCCCATCGAAACTGACCTCCAAA
ACCAAGCCTGCCAAGAAGTGTTGACCCCGGAAAACTCCAGGTA
CGTGGAAATGAAATCTCTGGAGGTGAGGTCACCAGAGTACACT
GAAGTGGAACTGAAACAGCCCCTTTCTTTGCCCTCTTGGGAAC
CAGAGGATGTGTTCAGTGAACTTAGCATTCCTCTAGGGGTGGA
GTTCGTGGTTCCCAGGACTGGCTTTTATTGCAAGCTGTGTGGG
CTGTTCTACACGAGCGAGGAGACAGCAAAGATGAGCCACTGCC
GCAGCGCTGTCCACTACAGGAACTTACAGAAATATTTGTCCCA
GCTGGCCGAGGAGGGCCTCAAGGAGACCGAGGGGGCAGATAGC
CCGAGGCCAGAGGACAGCGGAATCGTGCCACGCTTCGAAAGGA
AAAAGCTCTG
AAATAAAAGATCCTTATTTTCATTG
GATCTGTGTGTTGGTTTTTTGTGTG
aggaacccctagtgatggagttggccactccctctctgcgcgc
tcgctcgctcactgaggccgggcgaccaaaggtcgcccgacgc
ccgggctttgcccgggggcctcagtgagcgagcgagcgcgcag
agagggagtggccaa

In silico Construct 5 IS 5. scAAV with muscle creatine kinase (MCK) promoter and in silico derived Kozak sequence:
Lower case=5′ ITR
Underlined, uppercase=MCK promoter
Upper case, bold=in silico derived Kozak sequence
Upper case=RBM20 sequence
Upper case, bold underlined=PolyA
Lower case=3′ WT ITR

SEQ ID NO: 42
ttggccactccctctctgcgcgctcgctcgctcactgaggccggggaccaaaggtcgcccgacgcccgggctttgcccgggcggcctc
agtgagcgagcgagcgcgcagagagggagtggccaactccatcactaggggttcctCAAGGCTGTGGGGGACTGAG
GGCAGGCTGTAACAGGCTTGGGGGCCAGGGCTTATACGTGCCTGGGACTCCCAAAG
TATTACTGTTCCATGTTCCCGGCGAAGGGCCAGCTGTCCCCCGCCAGCTAGACTCAG
CACTTAGTTTAGGAACCAGTGAGCAAGTCAGCCCTTGGGGCAGCCCATACAAGGCC
ATGGGGCTGGGCAAGCTGCACGCCTGGGTCCGGGGTGGGCACGGTGCCCGGGCAAC
GAGCTGAAAGCTCATCTGCTCTCAGGGGCCCCTCCCTGGGGACAGCCCCTCCTGGCT
AGTCACACCCTGTAGGCTCCTCTATATAACCCAGGGGCACAGGGGCTGCCCTCAGC
CCCAACATGGTGCTGGCAGCAGCCATGAGCCAGGACGCGGACCCCAGCGGTCCGG
AGCAGCCGGACAGAGTTGCCTGCAGTGTGCCTGGTGCCCGGGCGTCCCCGGCACCC
TCCGGCCCGCGAGGGATGCAGCAGCCGCCGCCGCCGCCCCAGCCACCGCCCCCGCC
CCAAGCCGGCCTACCCCAGATCATCCAAAATGCCGCCAAGCTCCTGGACAAGAACC
CATTCTCGGTCAGTAACCCGAACCCTCTGCTTCCTTCACCTGCCAGTCTCCAGCTGGC
TCAACTGCAGGCCCAGCTCACCCTCCACCGGCTGAAGCTGGCACAGACAGCTGTCA
CCAACAACACTGCAGCCGCCACAGTCCTGAACCAAGTCCTCTCCAAAGTGGCCATGT
CCCAGCCTCTCTTCAATCAACTGAGGCATCCGTCTGTGATCACTGGCCCCCACGGCC
ATGCTGGGGTTCCCCAACATGCTGCAGCCATACCCAGTACCCGGTTTCCCTCTAATG
CAATTGCCTTTTCACCCCCCAGCCAGACACGAGGCCCCGGACCCTCCATGAACCTTC
CCAACCAGCCACCCAGTGCCATGGTGATGCATCCTTTCACTGGGGTAATGCCTCAGA
CCCCTGGCCAGCCAGCAGTCATCTTGGGCATTGGCAAGACTGGGCCTGCTCCAGCTA
CAGCAGGATTCTATGAGTATGGCAAAGCCAGCTCTGGCCAGACATATGGCCCTGAA
ACAGATGGTCAGCCTGGCTTCCTGCCATCCTCGGCCTCAACCTCGGGCAGTGTGACC
TATGAAGGGCACTACAGCCACACAGGGCAGGATGGTCAAGCTGCCTTTTCCAAAGA
TTTTTACGGACCCAACTCCCAAGGTTCACATGTGGCCAGCGGATTTCCAGCTGAGCA
GGCTGGGGGCCTGAAAAGTGAGGTCGGGCCACTGCTGCAGGGCACAAACAGCCAAT
GGGAGAGCCCCCATGGATTCTCGGGCCAAAGCAAGCCTGATCTCACAGCAGGTCCC
ATGTGGCCTCCACCCCACAACCAGCCCTATGAGCTGTACGACCCCGAGGAACCAAC
CTCAGACAGGACACCTCCTTCCTTCGGGGGTCGGCTTAACAACAGCAAACAGGGTTT
TATCGGTGCTGGGCGGAGGGCCAAGGAGGACCAGGCGTTGCTATCTGTGCGGCCTC
TGCAGGCTCATGAGCTGAACGACTTTCACGGTGTGGCCCCCCTCCACTTGCCGCATA
TCTGTAGCATCTGTGACAAGAAGGTGTTTGATTTGAAGGACTGGGAGCTGCATGTGA
AAGGGAAGCTGCACGCTCAGAAATGCCTGGTCTTCTCTGAAAATGCTGGCATCCGGT
GTATACTTGGTTCGGCAGAGGGAACATTGTGTGCTTCTCCCAACAGCACAGCTGTTT
ATAACCCTGCTGGGAATGAAGATTATGCCTCAAATCTTGGAACATCATACGTGCCCA
TTCCAGCAAGGTCATTCACTCAGTCAAGCCCCACATTTCCTTTGGCTTCTGTGGGGA
CAACTTTTGCACAGCGGAAAGGGGCTGGCCGTGTGGTGCACATCTGCAATCTCCCTG
AAGGAAGCTGCACTGAGAATGACGTCATTAACCTGGGGCTGCCCTTTGGAAAGGTC
ACTAATTACATCCTCATGAAATCGACTAATCAGGCCTTTTTAGAGATGGCTTACACA
GAAGCTGCACAGGCCATGGTCCAGTATTATCAAGAAAAATCTGCTGTGATCAATGG
TGAGAAGTTGCTCATTCGGATGTCCAAGAGATACAAGGAATTGCAGCTCAAGAAAC
CCGGGAAGGCCGTGGCTGCCATCATCCAGGACATCCATTCCCAGAGGGAGAGGGAC
ATGTTCCGGGAAGCAGACAGATATGGCCCAGAAAGGCCGCGGTCTCGTAGTCCGGT
GAGCCGGTCACTCTCCCCGAGGTCCCACACTCCCAGCTTCACCTCCTGCAGCTCTTC
CCACAGCCCTCCGGGCCCCTCCCGGGCTGACTGGGGCAATGGCCGGGACTCCTGGG
AGCACTCTCCCTATGCCAGGAGGGAGGAAGAGCGAGACCCGGCTCCCTGGAGGGAC
AACGGAGATGACAAGAGGGACAGGATGGACCCCTGGGCACATGATCGCAAACACC
ACCCCCGGCAACTGGACAAGGCTGAGTTGGACGAGCGACCAGAAGGAGGGAGGCC
CCACCGGGAGAAGTACCCGAGATCTGGGTCTCCCAACCTGCCCCACTCTGTGTCCAG
CTACAAAAGCCGTGAAGACGGCTACTACCGGAAAGAGCCCAAAGCCAAGTGGGAC
AAGTATCTGAAGCAGCAGCAGGATGCCCCCGGGAGGTCCAGGAGGAAAGACGAGG
CCAGGCTGCGGGAAAGCAGACACCCCCATCCGGATGACTCAGGCAAGGAAGATGG
GCTGGGGCCAAAGGTCACTAGGGCCCCTGAGGGCGCCAAGGCCAAGCAGAATGAG
AAAAATAAAACCAAGAGAACTGATAGAGACCAAGAAGGAGCTGATGATAGAAAAG
AAAACACAATGGCAGAGAATGAGGCTGGAAAAGAGGAACAGGAGGGCATGGAAGA
AAGCCCTCAATCAGTGGGCAGACAGGAGAAAGAAGCAGAGTTCTCTGATCCGGAAA
ACACAAGGACAAAGAAGGAACAAGATTGGGAGAGTGAAAGTGAGGCAGAGGGGG
AGAGCTGGTATCCCACTAACATGGAGGAGCTGGTGACAGTGGACGAGGTTGGGGAA
GAAGAAGATTTTATCGTGGAACCAGACATCCCAGAGCTGGAAGAAATTGTGCCCAT
TGACCAGAAAGACAAAATTTGCCCAGAAACATGTCTGTGTGTGACAACCACCTTAG
ACTTAGACCTGGCCCAGGATTTCCCCAAGGAAGGAGTCAAGGCCGTAGGGAATGGG
GCTGCAGAAATCAGCCTCAAGTCACCCAGAGAACTGCCCTCTGCTTCCACAAGCTGT
CCCAGTGACATGGACGTGGAAATGCCTGGCCTAAATCTGGATGCTGAGCGGAAGCC
AGCTGAAAGTGAGACAGGCCTCTCCCTGGAGGATTCAGATTGCTACGAGAAGGAGG
CAAAGGGAGTGGAGAGCTCAGATGTTCATCCAGCCCCTACAGTCCAGCAAATGTCT
TCCCCTAAGCCAGCAGAGGAGAGGGCCCGGCAGCCAAGCCCATTTGTGGATGATTG
CAAGACCAGGGGGACCCCCGAAGATGGGGCTTGTGAAGGCAGCCCCCTGGAGGAG
AAAGCCAGCCCCCCCATCGAAACTGACCTCCAAAACCAAGCCTGCCAAGAAGTGTT
GACCCCGGAAAACTCCAGGTACGTGGAAATGAAATCTCTGGAGGTGAGGTCACCAG
AGTACACTGAAGTGGAACTGAAACAGCCCCTTTCTTTGCCCTCTTGGGAACCAGAGG
ATGTGTTCAGTGAACTTAGCATTCCTCTAGGGGTGGAGTTCGTGGTTCCCAGGACTG
GCTTTTATTGCAAGCTGTGTGGGCTGTTCTACACGAGCGAGGAGACAGCAAAGATG
AGCCACTGCCGCAGCGCTGTCCACTACAGGAACTTACAGAAATATTTGTCCCAGCTG
GCCGAGGAGGGCCTCAAGGAGACCGAGGGGGCAGATAGCCCGAGGCCAGAGGACA
GCGGAATCGTGCCACGCTTCGAAAGGAAAAAGCTCTGAAATAAAAGATCCTTATTT
TCATTGGATCTGTGTGTTGGTTTTTTGTGTGaggaacccctagtgatggagttggccactccctctctgcg
cgctcgctcgctcactgaggccgggcgaccaaaggtcgcccgacgcccgggctttgcccgggcggcctcagtgagcgagcgagcgcg
cagagagggagtggccaa

In silico Construct 6 IS 6. scAAV with muscle creatine kinase (MCK) promoter and CAACCCAGC Kozak sequence:
Lower case=5′ ITR
Upper case, bold italics=spacer sequences
Underlined, uppercase=MCK promoter
Upper case, bold=Kozak sequence
Upper case=RBM20 sequence
Upper case, bold underlined=PolyA
Lower case=3′ WT ITR

SEQ ID NO: 43
ttggccactccctctctgcgcgctcgctcgctcactgaggccgggcgaccaaaggtcgcccgacgcccgggctttgcccgggcggcctc
agtgagcgagcgagcgcgcagagagggagtggccaactccatcactaggggttcctAAGGCTGTGGGGGACTGAGG
GCAGGCTGTAACAGGCTTGGGGGCCAGGGCTTATACGTGCCTGGGACTCCCAAAGT
ATTACTGTTCCATGTTCCCGGCGAAGGGCCAGCTGTCCCCCGCCAGCTAGACTCAGC
ACTTAGTTTAGGAACCAGTGAGCAAGTCAGCCCTTGGGGCAGCCCATACAAGGCCA
TGGGGCTGGGCAAGCTGCACGCCTGGGTCCGGGGTGGGCACGGTGCCCGGGCAACG
AGCTGAAAGCTCATCTGCTCTCAGGGGCCCCTCCCTGGGGACAGCCCCTCCTGGCTA
GTCACACCCTGTAGGCTCCTCTATATAACCCAGGGGCACAGGGGCTGCCCTCCAAC
CCAGCATGGTGCTGGCAGCAGCCATGAGCCAGGACGCGGACCCCAGCGGTCCGGA
GCAGCCGGACAGAGTTGCCTGCAGTGTGCCTGGTGCCCGGGCGTCCCCGGCACCCTC
CGGCCCGCGAGGGATGCAGCAGCCGCCGCCGCCGCCCCAGCCACCGCCCCCGCCCC
AAGCCGGCCTACCCCAGATCATCCAAAATGCCGCCAAGCTCCTGGACAAGAACCCA
TTCTCGGTCAGTAACCCGAACCCTCTGCTTCCTTCACCTGCCAGTCTCCAGCTGGCTC
AACTGCAGGCCCAGCTCACCCTCCACCGGCTGAAGCTGGCACAGACAGCTGTCACC
AACAACACTGCAGCCGCCACAGTCCTGAACCAAGTCCTCTCCAAAGTGGCCATGTCC
CAGCCTCTCTTCAATCAACTGAGGCATCCGTCTGTGATCACTGGCCCCCACGGCCAT
GCTGGGGTTCCCCAACATGCTGCAGCCATACCCAGTACCCGGTTTCCCTCTAATGCA
ATTGCCTTTTCACCCCCCAGCCAGACACGAGGCCCCGGACCCTCCATGAACCTTCCC
AACCAGCCACCCAGTGCCATGGTGATGCATCCTTTCACTGGGGTAATGCCTCAGACC
CCTGGCCAGCCAGCAGTCATCTTGGGCATTGGCAAGACTGGGCCTGCTCCAGCTACA
GCAGGATTCTATGAGTATGGCAAAGCCAGCTCTGGCCAGACATATGGCCCTGAAAC
AGATGGTCAGCCTGGCTTCCTGCCATCCTCGGCCTCAACCTCGGGCAGTGTGACCTA
TGAAGGGCACTACAGCCACACAGGGCAGGATGGTCAAGCTGCCTTTTCCAAAGATT
TTTACGGACCCAACTCCCAAGGTTCACATGTGGCCAGCGGATTTCCAGCTGAGCAGG
CTGGGGGCCTGAAAAGTGAGGTCGGGCCACTGCTGCAGGGCACAAACAGCCAATGG
GAGAGCCCCCATGGATTCTCGGGCCAAAGCAAGCCTGATCTCACAGCAGGTCCCAT
GTGGCCTCCACCCCACAACCAGCCCTATGAGCTGTACGACCCCGAGGAACCAACCT
CAGACAGGACACCTCCTTCCTTCGGGGGTCGGCTTAACAACAGCAAACAGGGTTTTA
TCGGTGCTGGGCGGAGGGCCAAGGAGGACCAGGCGTTGCTATCTGTGCGGCCTCTG
CAGGCTCATGAGCTGAACGACTTTCACGGTGTGGCCCCCCTCCACTTGCCGCATATC
TGTAGCATCTGTGACAAGAAGGTGTTTGATTTGAAGGACTGGGAGCTGCATGTGAA
AGGGAAGCTGCACGCTCAGAAATGCCTGGTCTTCTCTGAAAATGCTGGCATCCGGTG
TATACTTGGTTCGGCAGAGGGAACATTGTGTGCTTCTCCCAACAGCACAGCTGTTTA
TAACCCTGCTGGGAATGAAGATTATGCCTCAAATCTTGGAACATCATACGTGCCCAT
TCCAGCAAGGTCATTCACTCAGTCAAGCCCCACATTTCCTTTGGCTTCTGTGGGGAC
AACTTTTGCACAGCGGAAAGGGGCTGGCCGTGTGGTGCACATCTGCAATCTCCCTGA
AGGAAGCTGCACTGAGAATGACGTCATTAACCTGGGGCTGCCCTTTGGAAAGGTCA
CTAATTACATCCTCATGAAATCGACTAATCAGGCCTTTTTAGAGATGGCTTACACAG
AAGCTGCACAGGCCATGGTCCAGTATTATCAAGAAAAATCTGCTGTGATCAATGGT
GAGAAGTTGCTCATTCGGATGTCCAAGAGATACAAGGAATTGCAGCTCAAGAAACC
CGGGAAGGCCGTGGCTGCCATCATCCAGGACATCCATTCCCAGAGGGAGAGGGACA
TGTTCCGGGAAGCAGACAGATATGGCCCAGAAAGGCCGCGGTCTCGTAGTCCGGTG
AGCCGGTCACTCTCCCCGAGGTCCCACACTCCCAGCTTCACCTCCTGCAGCTCTTCCC
ACAGCCCTCCGGGCCCCTCCCGGGCTGACTGGGGCAATGGCCGGGACTCCTGGGAG
CACTCTCCCTATGCCAGGAGGGAGGAAGAGCGAGACCCGGCTCCCTGGAGGGACAA
CGGAGATGACAAGAGGGACAGGATGGACCCCTGGGCACATGATCGCAAACACCAC
CCCCGGCAACTGGACAAGGCTGAGTTGGACGAGCGACCAGAAGGAGGGAGGCCCC
ACCGGGAGAAGTACCCGAGATCTGGGTCTCCCAACCTGCCCCACTCTGTGTCCAGCT
ACAAAAGCCGTGAAGACGGCTACTACCGGAAAGAGCCCAAAGCCAAGTGGGACAA
GTATCTGAAGCAGCAGCAGGATGCCCCCGGGAGGTCCAGGAGGAAAGACGAGGCC
AGGCTGCGGGAAAGCAGACACCCCCATCCGGATGACTCAGGCAAGGAAGATGGGCT
GGGGCCAAAGGTCACTAGGGCCCCTGAGGGCGCCAAGGCCAAGCAGAATGAGAAA
AATAAAACCAAGAGAACTGATAGAGACCAAGAAGGAGCTGATGATAGAAAAGAAA
ACACAATGGCAGAGAATGAGGCTGGAAAAGAGGAACAGGAGGGCATGGAAGAAAG
CCCTCAATCAGTGGGCAGACAGGAGAAAGAAGCAGAGTTCTCTGATCCGGAAAACA
CAAGGACAAAGAAGGAACAAGATTGGGAGAGTGAAAGTGAGGCAGAGGGGGAGA
GCTGGTATCCCACTAACATGGAGGAGCTGGTGACAGTGGACGAGGTTGGGGAAGAA
GAAGATTTTATCGTGGAACCAGACATCCCAGAGCTGGAAGAAATTGTGCCCATTGA
CCAGAAAGACAAAATTTGCCCAGAAACATGTCTGTGTGTGACAACCACCTTAGACTT
AGACCTGGCCCAGGATTTCCCCAAGGAAGGAGTCAAGGCCGTAGGGAATGGGGCTG
CAGAAATCAGCCTCAAGTCACCCAGAGAACTGCCCTCTGCTTCCACAAGCTGTCCCA
GTGACATGGACGTGGAAATGCCTGGCCTAAATCTGGATGCTGAGCGGAAGCCAGCT
GAAAGTGAGACAGGCCTCTCCCTGGAGGATTCAGATTGCTACGAGAAGGAGGCAAA
GGGAGTGGAGAGCTCAGATGTTCATCCAGCCCCTACAGTCCAGCAAATGTCTTCCCC
TAAGCCAGCAGAGGAGAGGGCCCGGCAGCCAAGCCCATTTGTGGATGATTGCAAGA
CCAGGGGGACCCCCGAAGATGGGGCTTGTGAAGGCAGCCCCCTGGAGGAGAAAGC
CAGCCCCCCCATCGAAACTGACCTCCAAAACCAAGCCTGCCAAGAAGTGTTGACCC
CGGAAAACTCCAGGTACGTGGAAATGAAATCTCTGGAGGTGAGGTCACCAGAGTAC
ACTGAAGTGGAACTGAAACAGCCCCTTTCTTTGCCCTCTTGGGAACCAGAGGATGTG
TTCAGTGAACTTAGCATTCCTCTAGGGGTGGAGTTCGTGGTTCCCAGGACTGGCTTT
TATTGCAAGCTGTGTGGGCTGTTCTACACGAGCGAGGAGACAGCAAAGATGAGCCA
CTGCCGCAGCGCTGTCCACTACAGGAACTTACAGAAATATTTGTCCCAGCTGGCCGA
GGAGGGCCTCAAGGAGACCGAGGGGGCAGATAGCCCGAGGCCAGAGGACAGCGGA
ATCGTGCCACGCTTCGAAAGGAAAAAGCTCTGAAATAAAAGATCCTTATTTTCATT
GGATCTGTGTGTTGGTTTTTTGTGTGaggaacccctagtgatggagttggccactccctctctgcgcgctcgct
cgctcactgaggccgggcgaccaaaggtcgcccgacgcccgggctttgcccgggcggcctcagtgagcgagcgagcgcgcagagag
ggagtggccaa

In silico Construct 7 IS 7. scAAV with TNNC1 promoter and AGCGCCACC Kozak sequence:
Lower case=5′ ITR
Underlined, uppercase=TNNC1 promoter
Upper case, bold=Kozak sequence
Upper case=RBM20 sequence
Upper case, bold underlined=PolyA
Lower case=3′ WT ITR

SEQ ID NO: 44
ttggccactccctctctgcgcgctcgctcgctcactgaggccgggcgaccaaaggtcgcccgacgcccgggctttgcccgggcggcctc
agtgagcgagcgagcgcgcagagagggagtggccaactccatcactaggggttcctGATCACTGGGACCAGAGGAG
GGGCTGGAGGATACTACACGCAGGGGTGGGCTGGGCTGGGCTGGGCTGGGCCAGGA
ATGCAGCGGGGCAGGGCTATTTAAGTCAAGGGCCGGCTGGCAACCCCAGCAAGCTG
TCCTGTGAGAGCGCCACCATGGTGCTGGCAGCAGCCATGAGCCAGGACGCGGACC
CCAGCGGTCCGGAGCAGCCGGACAGAGTTGCCTGCAGTGTGCCTGGTGCCCGGGCG
TCCCCGGCACCCTCCGGCCCGCGAGGGATGCAGCAGCCGCCGCCGCCGCCCCAGCC
ACCGCCCCCGCCCCAAGCCGGCCTACCCCAGATCATCCAAAATGCCGCCAAGCTCCT
GGACAAGAACCCATTCTCGGTCAGTAACCCGAACCCTCTGCTTCCTTCACCTGCCAG
TCTCCAGCTGGCTCAACTGCAGGCCCAGCTCACCCTCCACCGGCTGAAGCTGGCACA
GACAGCTGTCACCAACAACACTGCAGCCGCCACAGTCCTGAACCAAGTCCTCTCCA
AAGTGGCCATGTCCCAGCCTCTCTTCAATCAACTGAGGCATCCGTCTGTGATCACTG
GCCCCCACGGCCATGCTGGGGTTCCCCAACATGCTGCAGCCATACCCAGTACCCGGT
TTCCCTCTAATGCAATTGCCTTTTCACCCCCCAGCCAGACACGAGGCCCCGGACCCT
CCATGAACCTTCCCAACCAGCCACCCAGTGCCATGGTGATGCATCCTTTCACTGGGG
TAATGCCTCAGACCCCTGGCCAGCCAGCAGTCATCTTGGGCATTGGCAAGACTGGGC
CTGCTCCAGCTACAGCAGGATTCTATGAGTATGGCAAAGCCAGCTCTGGCCAGACAT
ATGGCCCTGAAACAGATGGTCAGCCTGGCTTCCTGCCATCCTCGGCCTCAACCTCGG
GCAGTGTGACCTATGAAGGGCACTACAGCCACACAGGGCAGGATGGTCAAGCTGCC
TTTTCCAAAGATTTTTACGGACCCAACTCCCAAGGTTCACATGTGGCCAGCGGATTT
CCAGCTGAGCAGGCTGGGGGCCTGAAAAGTGAGGTCGGGCCACTGCTGCAGGGCAC
AAACAGCCAATGGGAGAGCCCCCATGGATTCTCGGGCCAAAGCAAGCCTGATCTCA
CAGCAGGTCCCATGTGGCCTCCACCCCACAACCAGCCCTATGAGCTGTACGACCCCG
AGGAACCAACCTCAGACAGGACACCTCCTTCCTTCGGGGGTCGGCTTAACAACAGC
AAACAGGGTTTTATCGGTGCTGGGCGGAGGGCCAAGGAGGACCAGGCGTTGCTATC
TGTGCGGCCTCTGCAGGCTCATGAGCTGAACGACTTTCACGGTGTGGCCCCCCTCCA
CTTGCCGCATATCTGTAGCATCTGTGACAAGAAGGTGTTTGATTTGAAGGACTGGGA
GCTGCATGTGAAAGGGAAGCTGCACGCTCAGAAATGCCTGGTCTTCTCTGAAAATG
CTGGCATCCGGTGTATACTTGGTTCGGCAGAGGGAACATTGTGTGCTTCTCCCAACA
GCACAGCTGTTTATAACCCTGCTGGGAATGAAGATTATGCCTCAAATCTTGGAACAT
CATACGTGCCCATTCCAGCAAGGTCATTCACTCAGTCAAGCCCCACATTTCCTTTGG
CTTCTGTGGGGACAACTTTTGCACAGCGGAAAGGGGCTGGCCGTGTGGTGCACATCT
GCAATCTCCCTGAAGGAAGCTGCACTGAGAATGACGTCATTAACCTGGGGCTGCCCT
TTGGAAAGGTCACTAATTACATCCTCATGAAATCGACTAATCAGGCCTTTTTAGAGA
TGGCTTACACAGAAGCTGCACAGGCCATGGTCCAGTATTATCAAGAAAAATCTGCT
GTGATCAATGGTGAGAAGTTGCTCATTCGGATGTCCAAGAGATACAAGGAATTGCA
GCTCAAGAAACCCGGGAAGGCCGTGGCTGCCATCATCCAGGACATCCATTCCCAGA
GGGAGAGGGACATGTTCCGGGAAGCAGACAGATATGGCCCAGAAAGGCCGCGGTC
TCGTAGTCCGGTGAGCCGGTCACTCTCCCCGAGGTCCCACACTCCCAGCTTCACCTC
CTGCAGCTCTTCCCACAGCCCTCCGGGCCCCTCCCGGGCTGACTGGGGCAATGGCCG
GGACTCCTGGGAGCACTCTCCCTATGCCAGGAGGGAGGAAGAGCGAGACCCGGCTC
CCTGGAGGGACAACGGAGATGACAAGAGGGACAGGATGGACCCCTGGGCACATGA
TCGCAAACACCACCCCCGGCAACTGGACAAGGCTGAGTTGGACGAGCGACCAGAAG
GAGGGAGGCCCCACCGGGAGAAGTACCCGAGATCTGGGTCTCCCAACCTGCCCCAC
TCTGTGTCCAGCTACAAAAGCCGTGAAGACGGCTACTACCGGAAAGAGCCCAAAGC
CAAGTGGGACAAGTATCTGAAGCAGCAGCAGGATGCCCCCGGGAGGTCCAGGAGG
AAAGACGAGGCCAGGCTGCGGGAAAGCAGACACCCCCATCCGGATGACTCAGGCA
AGGAAGATGGGCTGGGGCCAAAGGTCACTAGGGCCCCTGAGGGCGCCAAGGCCAA
GCAGAATGAGAAAAATAAAACCAAGAGAACTGATAGAGACCAAGAAGGAGCTGAT
GATAGAAAAGAAAACACAATGGCAGAGAATGAGGCTGGAAAAGAGGAACAGGAG
GGCATGGAAGAAAGCCCTCAATCAGTGGGCAGACAGGAGAAAGAAGCAGAGTTCT
CTGATCCGGAAAACACAAGGACAAAGAAGGAACAAGATTGGGAGAGTGAAAGTGA
GGCAGAGGGGGAGAGCTGGTATCCCACTAACATGGAGGAGCTGGTGACAGTGGAC
GAGGTTGGGGAAGAAGAAGATTTTATCGTGGAACCAGACATCCCAGAGCTGGAAGA
AATTGTGCCCATTGACCAGAAAGACAAAATTTGCCCAGAAACATGTCTGTGTGTGAC
AACCACCTTAGACTTAGACCTGGCCCAGGATTTCCCCAAGGAAGGAGTCAAGGCCG
TAGGGAATGGGGCTGCAGAAATCAGCCTCAAGTCACCCAGAGAACTGCCCTCTGCT
TCCACAAGCTGTCCCAGTGACATGGACGTGGAAATGCCTGGCCTAAATCTGGATGCT
GAGCGGAAGCCAGCTGAAAGTGAGACAGGCCTCTCCCTGGAGGATTCAGATTGCTA
CGAGAAGGAGGCAAAGGGAGTGGAGAGCTCAGATGTTCATCCAGCCCCTACAGTCC
AGCAAATGTCTTCCCCTAAGCCAGCAGAGGAGAGGGCCCGGCAGCCAAGCCCATTT
GTGGATGATTGCAAGACCAGGGGGACCCCCGAAGATGGGGCTTGTGAAGGCAGCCC
CCTGGAGGAGAAAGCCAGCCCCCCCATCGAAACTGACCTCCAAAACCAAGCCTGCC
AAGAAGTGTTGACCCCGGAAAACTCCAGGTACGTGGAAATGAAATCTCTGGAGGTG
AGGTCACCAGAGTACACTGAAGTGGAACTGAAACAGCCCCTTTCTTTGCCCTCTTGG
GAACCAGAGGATGTGTTCAGTGAACTTAGCATTCCTCTAGGGGTGGAGTTCGTGGTT
CCCAGGACTGGCTTTTATTGCAAGCTGTGTGGGCTGTTCTACACGAGCGAGGAGACA
GCAAAGATGAGCCACTGCCGCAGCGCTGTCCACTACAGGAACTTACAGAAATATTT
GTCCCAGCTGGCCGAGGAGGGCCTCAAGGAGACCGAGGGGGCAGATAGCCCGAGG
CCAGAGGACAGCGGAATCGTGCCACGCTTCGAAAGGAAAAAGCTCTGAAATAAAA
GATCCTTATTTTCATTGGATCTGTGTGTTGGTTTTTTGTGTGaggaacccctagtgatggagt
tggccactccctctctgcgcgctcgctcgctcactgaggccgggcgaccaaaggtcgcccgacgcccgggctttgcccgggcggcctca
gtgagcgagcgagcgcgcagagagggagtggccaa

In silico Construct 8 IS 8. scAAV withTNNC1 promoter and in silico derived Kozak sequence:
Lower case=5′ ITR
Underlined, uppercase=TNNC1 promoter
Upper case, bold=in silico derived Kozak sequence
Upper case=RBM20 sequence
Upper case, bold underlined=PolyA
Lower case=3′ WT ITR

SEQ ID NO: 45
ttggccactccctctctgcgcgctcgctcgctcactgaggccgggcgaccaaaggtcgcccgacgcccgggctttgcccgggcggcctc
agtgagcgagcgagcgcgcagagagggagtggccaactccatcactaggggttcctGATCACTGGGACCAGAGGAG
GGGCTGGAGGATACTACACGCAGGGGTGGGCTGGGCTGGGCTGGGCTGGGCCAGGA
ATGCAGCGGGGCAGGGCTATTTAAGTCAAGGGCCGGCTGGCAACCCCAGCAAGCTG
TCCTGTGAGAGCCCCAACATGGTGCTGGCAGCAGCCATGAGCCAGGACGCGGACCC
CAGCGGTCCGGAGCAGCCGGACAGAGTTGCCTGCAGTGTGCCTGGTGCCCGGGCGT
CCCCGGCACCCTCCGGCCCGCGAGGGATGCAGCAGCCGCCGCCGCCGCCCCAGCCA
CCGCCCCCGCCCCAAGCCGGCCTACCCCAGATCATCCAAAATGCCGCCAAGCTCCTG
GACAAGAACCCATTCTCGGTCAGTAACCCGAACCCTCTGCTTCCTTCACCTGCCAGT
CTCCAGCTGGCTCAACTGCAGGCCCAGCTCACCCTCCACCGGCTGAAGCTGGCACAG
ACAGCTGTCACCAACAACACTGCAGCCGCCACAGTCCTGAACCAAGTCCTCTCCAA
AGTGGCCATGTCCCAGCCTCTCTTCAATCAACTGAGGCATCCGTCTGTGATCACTGG
CCCCCACGGCCATGCTGGGGTTCCCCAACATGCTGCAGCCATACCCAGTACCCGGTT
TCCCTCTAATGCAATTGCCTTTTCACCCCCCAGCCAGACACGAGGCCCCGGACCCTC
CATGAACCTTCCCAACCAGCCACCCAGTGCCATGGTGATGCATCCTTTCACTGGGGT
AATGCCTCAGACCCCTGGCCAGCCAGCAGTCATCTTGGGCATTGGCAAGACTGGGC
CTGCTCCAGCTACAGCAGGATTCTATGAGTATGGCAAAGCCAGCTCTGGCCAGACAT
ATGGCCCTGAAACAGATGGTCAGCCTGGCTTCCTGCCATCCTCGGCCTCAACCTCGG
GCAGTGTGACCTATGAAGGGCACTACAGCCACACAGGGCAGGATGGTCAAGCTGCC
TTTTCCAAAGATTTTTACGGACCCAACTCCCAAGGTTCACATGTGGCCAGCGGATTT
CCAGCTGAGCAGGCTGGGGGCCTGAAAAGTGAGGTCGGGCCACTGCTGCAGGGCAC
AAACAGCCAATGGGAGAGCCCCCATGGATTCTCGGGCCAAAGCAAGCCTGATCTCA
CAGCAGGTCCCATGTGGCCTCCACCCCACAACCAGCCCTATGAGCTGTACGACCCCG
AGGAACCAACCTCAGACAGGACACCTCCTTCCTTCGGGGGTCGGCTTAACAACAGC
AAACAGGGTTTTATCGGTGCTGGGCGGAGGGCCAAGGAGGACCAGGCGTTGCTATC
TGTGCGGCCTCTGCAGGCTCATGAGCTGAACGACTTTCACGGTGTGGCCCCCCTCCA
CTTGCCGCATATCTGTAGCATCTGTGACAAGAAGGTGTTTGATTTGAAGGACTGGGA
GCTGCATGTGAAAGGGAAGCTGCACGCTCAGAAATGCCTGGTCTTCTCTGAAAATG
CTGGCATCCGGTGTATACTTGGTTCGGCAGAGGGAACATTGTGTGCTTCTCCCAACA
GCACAGCTGTTTATAACCCTGCTGGGAATGAAGATTATGCCTCAAATCTTGGAACAT
CATACGTGCCCATTCCAGCAAGGTCATTCACTCAGTCAAGCCCCACATTTCCTTTGG
CTTCTGTGGGGACAACTTTTGCACAGCGGAAAGGGGCTGGCCGTGTGGTGCACATCT
GCAATCTCCCTGAAGGAAGCTGCACTGAGAATGACGTCATTAACCTGGGGCTGCCCT
TTGGAAAGGTCACTAATTACATCCTCATGAAATCGACTAATCAGGCCTTTTTAGAGA
TGGCTTACACAGAAGCTGCACAGGCCATGGTCCAGTATTATCAAGAAAAATCTGCT
GTGATCAATGGTGAGAAGTTGCTCATTCGGATGTCCAAGAGATACAAGGAATTGCA
GCTCAAGAAACCCGGGAAGGCCGTGGCTGCCATCATCCAGGACATCCATTCCCAGA
GGGAGAGGGACATGTTCCGGGAAGCAGACAGATATGGCCCAGAAAGGCCGCGGTC
TCGTAGTCCGGTGAGCCGGTCACTCTCCCCGAGGTCCCACACTCCCAGCTTCACCTC
CTGCAGCTCTTCCCACAGCCCTCCGGGCCCCTCCCGGGCTGACTGGGGCAATGGCCG
GGACTCCTGGGAGCACTCTCCCTATGCCAGGAGGGAGGAAGAGCGAGACCCGGCTC
CCTGGAGGGACAACGGAGATGACAAGAGGGACAGGATGGACCCCTGGGCACATGA
TCGCAAACACCACCCCCGGCAACTGGACAAGGCTGAGTTGGACGAGCGACCAGAAG
GAGGGAGGCCCCACCGGGAGAAGTACCCGAGATCTGGGTCTCCCAACCTGCCCCAC
TCTGTGTCCAGCTACAAAAGCCGTGAAGACGGCTACTACCGGAAAGAGCCCAAAGC
CAAGTGGGACAAGTATCTGAAGCAGCAGCAGGATGCCCCCGGGAGGTCCAGGAGG
AAAGACGAGGCCAGGCTGCGGGAAAGCAGACACCCCCATCCGGATGACTCAGGCA
AGGAAGATGGGCTGGGGCCAAAGGTCACTAGGGCCCCTGAGGGCGCCAAGGCCAA
GCAGAATGAGAAAAATAAAACCAAGAGAACTGATAGAGACCAAGAAGGAGCTGAT
GATAGAAAAGAAAACACAATGGCAGAGAATGAGGCTGGAAAAGAGGAACAGGAG
GGCATGGAAGAAAGCCCTCAATCAGTGGGCAGACAGGAGAAAGAAGCAGAGTTCT
CTGATCCGGAAAACACAAGGACAAAGAAGGAACAAGATTGGGAGAGTGAAAGTGA
GGCAGAGGGGGAGAGCTGGTATCCCACTAACATGGAGGAGCTGGTGACAGTGGAC
GAGGTTGGGGAAGAAGAAGATTTTATCGTGGAACCAGACATCCCAGAGCTGGAAGA
AATTGTGCCCATTGACCAGAAAGACAAAATTTGCCCAGAAACATGTCTGTGTGTGAC
AACCACCTTAGACTTAGACCTGGCCCAGGATTTCCCCAAGGAAGGAGTCAAGGCCG
TAGGGAATGGGGCTGCAGAAATCAGCCTCAAGTCACCCAGAGAACTGCCCTCTGCT
TCCACAAGCTGTCCCAGTGACATGGACGTGGAAATGCCTGGCCTAAATCTGGATGCT
GAGCGGAAGCCAGCTGAAAGTGAGACAGGCCTCTCCCTGGAGGATTCAGATTGCTA
CGAGAAGGAGGCAAAGGGAGTGGAGAGCTCAGATGTTCATCCAGCCCCTACAGTCC
AGCAAATGTCTTCCCCTAAGCCAGCAGAGGAGAGGGCCCGGCAGCCAAGCCCATTT
GTGGATGATTGCAAGACCAGGGGGACCCCCGAAGATGGGGCTTGTGAAGGCAGCCC
CCTGGAGGAGAAAGCCAGCCCCCCCATCGAAACTGACCTCCAAAACCAAGCCTGCC
AAGAAGTGTTGACCCCGGAAAACTCCAGGTACGTGGAAATGAAATCTCTGGAGGTG
AGGTCACCAGAGTACACTGAAGTGGAACTGAAACAGCCCCTTTCTTTGCCCTCTTGG
GAACCAGAGGATGTGTTCAGTGAACTTAGCATTCCTCTAGGGGTGGAGTTCGTGGTT
CCCAGGACTGGCTTTTATTGCAAGCTGTGTGGGCTGTTCTACACGAGCGAGGAGACA
GCAAAGATGAGCCACTGCCGCAGCGCTGTCCACTACAGGAACTTACAGAAATATTT
GTCCCAGCTGGCCGAGGAGGGCCTCAAGGAGACCGAGGGGGCAGATAGCCCGAGG
CCAGAGGACAGCGGAATCGTGCCACGCTTCGAAAGGAAAAAGCTCTGAAATAAAA
GATCCTTATTTTCATTGGATCTGTGTGTTGGTTTTTTGTGTGaggaacccctagtgatggagt
tggccactccctctctgcgcgctcgctcgctcactgaggccgggcgaccaaaggtcgcccgacgcccgggctttgcccgggcggcctca
gtgagcgagcgagcgcgcagagagggagtggccaa

In silico Construct 9 IS 9. scAAV with TNNC1 promoter and CAACCCAGC Kozak sequence:
Lower case=5′ ITR
Upper case, bold italics=spacer sequences
Underlined, uppercase=TNNC1 promoter
Upper case, bold=Kozak sequence
Upper case=RBM20 sequence
Upper case, bold underlined=PolyA
Lower case=3′ WT ITR

SEQ ID NO: 46
ttggccactccctctctgcgcgctcgctcgctcactgaggccggggaccaaaggtcgcccgacgcccgggctttgcccgggggcctc
agtgagcgagcgagcgcgcagagagggagtggccaactccatcactaggggttcctGATCACTGGGACCAGAGGAG
GGGCTGGAGGATACTACACGCAGGGGTGGGCTGGGCTGGGCTGGGCTGGGCCAGGA
ATGCAGCGGGGCAGGGCTATTTAAGTCAAGGGCCGGCTGGCAACCCCAGCAAGCTG
TCCTGTGAGCAACCCAGCATGGTGCTGGCAGCAGCCATGAGCCAGGACGCGGACCC
CAGCGGTCCGGAGCAGCCGGACAGAGTTGCCTGCAGTGTGCCTGGTGCCCGGGCGT
CCCCGGCACCCTCCGGCCCGCGAGGGATGCAGCAGCCGCCGCCGCCGCCCCAGCCA
CCGCCCCCGCCCCAAGCCGGCCTACCCCAGATCATCCAAAATGCCGCCAAGCTCCTG
GACAAGAACCCATTCTCGGTCAGTAACCCGAACCCTCTGCTTCCTTCACCTGCCAGT
CTCCAGCTGGCTCAACTGCAGGCCCAGCTCACCCTCCACCGGCTGAAGCTGGCACAG
ACAGCTGTCACCAACAACACTGCAGCCGCCACAGTCCTGAACCAAGTCCTCTCCAA
AGTGGCCATGTCCCAGCCTCTCTTCAATCAACTGAGGCATCCGTCTGTGATCACTGG
CCCCCACGGCCATGCTGGGGTTCCCCAACATGCTGCAGCCATACCCAGTACCCGGTT
TCCCTCTAATGCAATTGCCTTTTCACCCCCCAGCCAGACACGAGGCCCCGGACCCTC
CATGAACCTTCCCAACCAGCCACCCAGTGCCATGGTGATGCATCCTTTCACTGGGGT
AATGCCTCAGACCCCTGGCCAGCCAGCAGTCATCTTGGGCATTGGCAAGACTGGGC
CTGCTCCAGCTACAGCAGGATTCTATGAGTATGGCAAAGCCAGCTCTGGCCAGACAT
ATGGCCCTGAAACAGATGGTCAGCCTGGCTTCCTGCCATCCTCGGCCTCAACCTCGG
GCAGTGTGACCTATGAAGGGCACTACAGCCACACAGGGCAGGATGGTCAAGCTGCC
TTTTCCAAAGATTTTTACGGACCCAACTCCCAAGGTTCACATGTGGCCAGCGGATTT
CCAGCTGAGCAGGCTGGGGGCCTGAAAAGTGAGGTCGGGCCACTGCTGCAGGGCAC
AAACAGCCAATGGGAGAGCCCCCATGGATTCTCGGGCCAAAGCAAGCCTGATCTCA
CAGCAGGTCCCATGTGGCCTCCACCCCACAACCAGCCCTATGAGCTGTACGACCCCG
AGGAACCAACCTCAGACAGGACACCTCCTTCCTTCGGGGGTCGGCTTAACAACAGC
AAACAGGGTTTTATCGGTGCTGGGCGGAGGGCCAAGGAGGACCAGGCGTTGCTATC
TGTGCGGCCTCTGCAGGCTCATGAGCTGAACGACTTTCACGGTGTGGCCCCCCTCCA
CTTGCCGCATATCTGTAGCATCTGTGACAAGAAGGTGTTTGATTTGAAGGACTGGGA
GCTGCATGTGAAAGGGAAGCTGCACGCTCAGAAATGCCTGGTCTTCTCTGAAAATG
CTGGCATCCGGTGTATACTTGGTTCGGCAGAGGGAACATTGTGTGCTTCTCCCAACA
GCACAGCTGTTTATAACCCTGCTGGGAATGAAGATTATGCCTCAAATCTTGGAACAT
CATACGTGCCCATTCCAGCAAGGTCATTCACTCAGTCAAGCCCCACATTTCCTTTGG
CTTCTGTGGGGACAACTTTTGCACAGCGGAAAGGGGCTGGCCGTGTGGTGCACATCT
GCAATCTCCCTGAAGGAAGCTGCACTGAGAATGACGTCATTAACCTGGGGCTGCCCT
TTGGAAAGGTCACTAATTACATCCTCATGAAATCGACTAATCAGGCCTTTTTAGAGA
TGGCTTACACAGAAGCTGCACAGGCCATGGTCCAGTATTATCAAGAAAAATCTGCT
GTGATCAATGGTGAGAAGTTGCTCATTCGGATGTCCAAGAGATACAAGGAATTGCA
GCTCAAGAAACCCGGGAAGGCCGTGGCTGCCATCATCCAGGACATCCATTCCCAGA
GGGAGAGGGACATGTTCCGGGAAGCAGACAGATATGGCCCAGAAAGGCCGCGGTC
TCGTAGTCCGGTGAGCCGGTCACTCTCCCCGAGGTCCCACACTCCCAGCTTCACCTC
CTGCAGCTCTTCCCACAGCCCTCCGGGCCCCTCCCGGGCTGACTGGGGCAATGGCCG
GGACTCCTGGGAGCACTCTCCCTATGCCAGGAGGGAGGAAGAGCGAGACCCGGCTC
CCTGGAGGGACAACGGAGATGACAAGAGGGACAGGATGGACCCCTGGGCACATGA
TCGCAAACACCACCCCCGGCAACTGGACAAGGCTGAGTTGGACGAGCGACCAGAAG
GAGGGAGGCCCCACCGGGAGAAGTACCCGAGATCTGGGTCTCCCAACCTGCCCCAC
TCTGTGTCCAGCTACAAAAGCCGTGAAGACGGCTACTACCGGAAAGAGCCCAAAGC
CAAGTGGGACAAGTATCTGAAGCAGCAGCAGGATGCCCCCGGGAGGTCCAGGAGG
AAAGACGAGGCCAGGCTGCGGGAAAGCAGACACCCCCATCCGGATGACTCAGGCA
AGGAAGATGGGCTGGGGCCAAAGGTCACTAGGGCCCCTGAGGGCGCCAAGGCCAA
GCAGAATGAGAAAAATAAAACCAAGAGAACTGATAGAGACCAAGAAGGAGCTGAT
GATAGAAAAGAAAACACAATGGCAGAGAATGAGGCTGGAAAAGAGGAACAGGAG
GGCATGGAAGAAAGCCCTCAATCAGTGGGCAGACAGGAGAAAGAAGCAGAGTTCT
CTGATCCGGAAAACACAAGGACAAAGAAGGAACAAGATTGGGAGAGTGAAAGTGA
GGCAGAGGGGGAGAGCTGGTATCCCACTAACATGGAGGAGCTGGTGACAGTGGAC
GAGGTTGGGGAAGAAGAAGATTTTATCGTGGAACCAGACATCCCAGAGCTGGAAGA
AATTGTGCCCATTGACCAGAAAGACAAAATTTGCCCAGAAACATGTCTGTGTGTGAC
AACCACCTTAGACTTAGACCTGGCCCAGGATTTCCCCAAGGAAGGAGTCAAGGCCG
TAGGGAATGGGGCTGCAGAAATCAGCCTCAAGTCACCCAGAGAACTGCCCTCTGCT
TCCACAAGCTGTCCCAGTGACATGGACGTGGAAATGCCTGGCCTAAATCTGGATGCT
GAGCGGAAGCCAGCTGAAAGTGAGACAGGCCTCTCCCTGGAGGATTCAGATTGCTA
CGAGAAGGAGGCAAAGGGAGTGGAGAGCTCAGATGTTCATCCAGCCCCTACAGTCC
AGCAAATGTCTTCCCCTAAGCCAGCAGAGGAGAGGGCCCGGCAGCCAAGCCCATTT
GTGGATGATTGCAAGACCAGGGGGACCCCCGAAGATGGGGCTTGTGAAGGCAGCCC
CCTGGAGGAGAAAGCCAGCCCCCCCATCGAAACTGACCTCCAAAACCAAGCCTGCC
AAGAAGTGTTGACCCCGGAAAACTCCAGGTACGTGGAAATGAAATCTCTGGAGGTG
AGGTCACCAGAGTACACTGAAGTGGAACTGAAACAGCCCCTTTCTTTGCCCTCTTGG
GAACCAGAGGATGTGTTCAGTGAACTTAGCATTCCTCTAGGGGTGGAGTTCGTGGTT
CCCAGGACTGGCTTTTATTGCAAGCTGTGTGGGCTGTTCTACACGAGCGAGGAGACA
GCAAAGATGAGCCACTGCCGCAGCGCTGTCCACTACAGGAACTTACAGAAATATTT
GTCCCAGCTGGCCGAGGAGGGCCTCAAGGAGACCGAGGGGGCAGATAGCCCGAGG
CCAGAGGACAGCGGAATCGTGCCACGCTTCGAAAGGAAAAAGCTCTGAAATAAAA
GATCCTTATTTTCATTGGATCTGTGTGTTGGTTTTTTGTGTGaggaacccctagtgatggagt
tggccactccctctctgcgcgctcgctcgctcactgaggccgggcgaccaaaggtcgcccgacgcccgggctttgcccgggggcctca
gtgagcgagcgagcgcgcagagagggagtggccaa

In some embodiments, the promoter, Kozak sequence, and transgene from Tables 2-4 below may be assembled into an exemplary construct, wherein the exemplary construct comprises at least one promoter, at least one Kozak, and at least one transgene. For example, a Desmin (Des1) promoter, an in silico derived Kozak Sequence, and RBM20 may be placed within an exemplary construct.

TABLE 2

| | |
|---|---|
| Promoter | Herpes Simplex virus (HSV) |
| | Thymidine kinase (TK) |
| | Rous Sarcoma Virus (RSV) |
| | Simian Virus 40 (SV40) |
| | Mouse Mammary Tumor Virus (MMTV) |
| | Ad E1A and cytomegalovirus (CMV) promoters |
| | chicken β-actin promoter (CBA) |
| | Desmin |
| | Muscle Creatine Kinase (MCK) |
| | TNNT2 |

TABLE 3

| Kozak | Native to RBM20 |
|---|---|
| | Canonical Kozak |
| | (e.g., GCCACC (SEQ ID NO: 47)) |
| | In silico consensus |
| | (e.g., AGCCCCAAC (SEQ ID NO: 36)) |

TABLE 4

| | |
|---|---|
| Transgene or | Human RBM20 (e.g., comprising SEQ ID NO: |
| Protein to be | NO. 5 or encoding SEQ ID NO: 8) |
| Expressed | |

### Pharmaceutical Formulations and Administration

Compositions described herein may further comprise a pharmaceutical excipient, buffer, or diluent, and may be formulated for administration to host cell ex vivo or in situ in an animal, and particularly a human being. Such compositions may further optionally comprise a liposome, a lipid, a lipid complex, a microsphere, a microparticle, a nanosphere, or a nanoparticle, or may be otherwise formulated for administration to the cells, tissues, organs, or body of a subject in need thereof. Such compositions may be formulated for use in a variety of therapies, such as for example, in the amelioration, prevention, and/or treatment of conditions such as peptide deficiency, polypeptide deficiency, peptide overexpression, polypeptide overexpression, including for example, conditions which result in diseases or disorders as described herein.

Formulations comprising pharmaceutically-acceptable excipients and/or carrier solutions arc well-known to those of skill in the art, as is the development of suitable dosing and treatment regimens for using the particular compositions described herein in a variety of treatment regimens, including e.g., oral, parenteral, intravenous, intranasal, intra-articular, and intramuscular administration and formulation.

Typically, these formulations may contain at least about 0.1% of the therapeutic agent (e.g., therapeutic rAAV particle or preparation) or more, although the percentage of the active ingredient(s) may, of course, be varied and may conveniently be between about 1 or 2% and about 70% or 90% or more of the weight or volume of the total formulation. Naturally, the amount of therapeutic agent(s) in each therapeutically-useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art when preparing such pharmaceutical formulations. Additionally, a variety of dosages and treatment regimens may be desirable.

In certain circumstances, it will be desirable to deliver the therapeutic rAAV particles or preparations in suitably formulated pharmaceutical compositions disclosed herein; either subcutaneously, intracardially, intraocularly, intravitreally, parenterally, subcutaneously, intravenously, intracerebro-ventricularly, intramuscularly, intrathecally, orally, intraperitoneally, by oral or nasal inhalation, or by direct injection to one or more cells (e.g., cardiomyocytes and/or other heart cells), tissues, or organs. In some embodiments, the therapeutic rAAV particles or the composition comprising the therapeutic rAAV particles of the present invention are delivered systemically via intravenous injection, particularly in those for treating a human. In some embodiments, the therapeutic rAAV particles or the composition comprising the therapeutic rAAV particles of the present invention are injected directly into the heart of the subject. Direct injection to the heart may comprise injection into one or more of the myocardial tissues, the cardiac lining, or the skeletal muscle surrounding the heart, e.g., using a needle catheter. In several embodiments, direct injection to human heart is preferred, for example, if delivery is performed concurrently with a surgical procedure or interventional procedure whereby access to the heart is improved. In some embodiments, the interventional procedure includes any procedure wherein coronary or pulmonary perfusion is altered. In some embodiments, the interventional procedure includes one or more of percutaneous administration, catheterization, or coronary retroperfusion.

The pharmaceutical formulations of the compositions suitable for injectable usc include sterile aqueous solutions or dispersions. In some embodiments, the formulation is sterile and fluid to the extent that easy syringability exists. In some embodiments, the form is stable under the conditions of manufacture and storage, and is preserved against the contaminating action of microorganisms, such as bacteria and fungi. The carrier may be a solvent or dispersion medium containing, for example, water, saline, ethanol, polyol (e.g., glycerol, propylene glycol, and liquid polyethylene glycol, and the like), suitable mixtures thereof, vegetable oils or other pharmaceutically acceptable carriers such as those that are Generally Recognized as Safe (GRAS) by the United States Food and Drug Administration. Proper fluidity may be maintained, for example, by the use of a coating, such as lecithin, by the maintenance of the required particle size in the case of dispersion and by the use of surfactants. In fact, there is virtually no limit to other components that may also be included, as long as the additional agents do not cause a significant adverse effect upon contact with the target cells or host tissues. The therapeutic rAAV particles or preparations may thus be delivered along with various other pharmaceutically acceptable agents as required in the particular instance. Such compositions may be purified from host cells or other biological sources, or alternatively may be chemically synthesized as described herein.

The amount of therapeutic rAAV particle or preparation, and/or therapeutic rAAV vector compositions and time of administration of such compositions will be within the purview of the skilled artisan having benefit of the present teachings. It is likely, however, that the administration of therapeutically-effective amounts of the compositions of the present disclosure may be achieved by a single administration, such as for example, a single injection of sufficient numbers of infectious particles to provide therapeutic benefit to the patient undergoing such treatment. In some circumstances, it may be desirable to provide multiple or successive administrations of the rAAV particle or preparation, and/or rAAV vector compositions, either over a relatively short, or a relatively prolonged period of time, as may be determined by the medical practitioner overseeing the administration of such compositions.

Toxicity and efficacy of the compositions utilized in methods of the present invention may be determined by standard pharmaceutical procedures, using either cells in culture or experimental animals to determine the LD₅₀ (the dose lethal to 50% of the population). The dose ratio between toxicity and efficacy the therapeutic index and it may be expressed as the ratio LD₅₀/ED₅₀. Those compositions that exhibit large therapeutic indices are preferred. While compositions that exhibit toxic side effects may be used, care should be taken to design a delivery system that minimizes the potential damage of such side effects. The dosage of compositions as described herein lies generally within a range that includes an ED₅₀ with little or no toxicity. The dosage may vary within this range depending upon the dosage form employed and the route of administration utilized.

Other aspects of the present disclosure relate to methods and preparations for use with a subject, such as human or non-human subjects, a host cell in situ in a subject, or a host cell derived from a subject. In some embodiments, the subject is a mammal. In some embodiments, the subject is a companion animal. “A companion animal”, as used herein, refers to pets and other domestic animals. Non-limiting examples of companion animals include dogs and cats; livestock such as horses, cattle, pigs, sheep, goats, and chickens; and other animals such as mice, rats, guinea pigs, and hamsters. In some embodiments, the subject is a human subject.

In some embodiments, one or more pharmaceutically acceptable excipients (including vehicles, carriers, diluents, and/or delivery polymers) are added to the pharmaceutical compositions including a therapeutic, thereby forming a pharmaceutical formulation suitable for in vivo delivery to a subject, such as a human.

A pharmaceutical composition or medicament includes a pharmacologically effective amount of at least one of the therapeutic and optionally one or more pharmaceutically acceptable excipients. Pharmaceutically acceptable excipients (excipients) are substances other than the Active Pharmaceutical ingredient (API, therapeutic product) that are intentionally included in the drug delivery system. Excipients do not exert or are not intended to exert a therapeutic effect at the intended dosage. Excipients may act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

The pharmaceutical compositions can contain other additional components commonly found in pharmaceutical compositions. Such additional components can include, but are not limited to: anti-pruritics, astringents, local anesthetics, or anti-inflammatory agents (e.g., antihistamine, diphenhydramine, etc.).

The carrier can be, but is not limited to, a solvent or dispersion medium containing, for example, water, ethanol, polyol (for example, glycerol, propylene glycol, and liquid polyethylene glycol), and suitable mixtures thereof. A carrier may also contain adjuvants such as preservatives, wetting agents, emulsifying agents, and dispersing agents. A carrier may also contain isotonic agents, such as sugars, polyalcohols, sodium chloride, and the like into the compositions.

Pharmaceutically acceptable refers to those properties and/or substances which are acceptable to the subject from a pharmacological/toxicological point of view. The phrase pharmaceutically acceptable refers to molecular entities, compositions, and properties that are physiologically tolerable and do not typically produce an allergic or other untoward or toxic reaction when administered to a subject. In some embodiments, a pharmaceutically acceptable compound is approved by a regulatory agency of the Federal or a state government or listed in the U.S. Pharmacopeia or other generally recognized pharmacopeia for use in animals and more particularly in humans.

The rAAVs or pharmaceutical compositions as described herein, may be formulated for administration to host cell ex vivo or in situ in an animal, and particularly a human being. The rAAVs or pharmaceutical compositions can be administered by a variety of routes. Administration routes included, but are not limited to, intravenous, intra-arterial, subcutaneous, intramuscular, intrahepatic, intraperitoneal and/or local delivery to a target tissue. In some embodiments, a plurality of injections, or other administration types, are provided, for example 2, 3, 4, 5, 6, 7, 8, 9, 10 or more injections. Routes of administration may be combined, if desired. Depending on the embodiment, the first and second rAAV need not be administered the same number of times (e.g., the first rAAV may be administered 1 time, and the second vector may be administered three times). In some embodiments, the dosing is intramuscular administration.

In some embodiments, the number of rAAV particles administered to a subject may be on the order ranging from about 10⁶ to about 10¹⁴ particles/mL or about 10³ to about 10¹³ particles/mL, or any values in between for either range, such as for example, about 10⁶, 10⁷, 10⁸, 10⁹, 10¹⁰, 10¹¹, 10¹², 10¹³, or 10¹⁴ particles/mL. In some embodiments, the number of rAAV particles administered to a subject may be on the order ranging from about 10⁶ to about 10¹⁴ vector genomes (vgs)/mL or 10³ to 10¹⁵ vgs/mL, or any values in between for either range, such as for example, about 10⁶, 10⁷, 10⁸, 10⁹, 10¹⁰, 10¹¹, 10¹², 10¹³, or 10¹⁴ vgs/mL. The rAAV particles can be administered as a single dose, or divided into two or more administrations as may be required to achieve therapy of the particular disease or disorder being treated. In some embodiments, doses ranging from about 0.0001 mL to about 10 mL are delivered to a subject.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, intravitreal, subcutaneous and intraperitoneal administration. In this connection, a sterile aqueous medium that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion, (see, for example, “Remington's Pharmaceutical Sciences” 15th Edition, pages 1035-1038 and 1570-1580). In several embodiments, the rAAV formulation will comprise, consist of, or consist essentially of active rAAV ingredient, a mono-basic buffer (e.g., sodium phosphate mono-basic buffer, a di-basic salt (e.g., sodium phosphate di-basic), a sodium-based tonicifier (e.g., sodium chloride tonicifier), a non-sodium tonicifier (e.g., magnesium chloride hexahydrate tonicifier), a surfactant (e.g., poloxamer 188 surfactant), and water. In several embodiments, the rAAV formulation will comprise, consist of, or consist essentially of active rAAV ingredient, sodium phosphate mono-basic buffer, sodium phosphate di-based, sodium chloride tonicifier, magnesium chloride hexahydrate tonicifier, poloxamer 188 surfactant, and water. In several embodiments, the active rAAV ingredient is present in the formulation according to the vector genome amounts provided for herein. In several embodiments, the mono-basic buffer (e.g., sodium phosphate mono-basic buffer) is present in the formulation at a concentration between about 0.2 mg/mL and about 0.5 mg/mL. In several embodiments, the di-basic salt (e.g., sodium phosphate di-basic) is present in the formulation at a concentration between about 1.5 mg/mL and about 4 mg/mL. In several embodiments, the sodium-based tonicifier (e.g., sodium chloride tonicifier) is present in the formulation at a concentration between about 8 mg/mL and about 12 mg/mL. In several embodiments, the non-sodium tonicifier (e.g., magnesium chloride hexahydrate tonicifier) is present in the formulation at a concentration between about 0.1 mg/mL and about 0.35 mg/mL. In several embodiments, the surfactant (e.g., poloxamer 188 surfactant) is present in the formulation at a concentration between about 0.05 mg/ml and about 0.8 mg/mL. In several embodiments, water is present to bring the volume of the formulation (e.g., a dosage unit) to 1 mL.

Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by, e.g., FDA Office of Biologics standards.

Sterile injectable solutions are prepared by incorporating the rAAV particles or preparations in the required amount in the appropriate solvent with several of the other ingredients enumerated above, as required, followed by filtered sterilization. Generally, dispersions are prepared by incorporating the various sterilized active ingredients into a sterile vehicle that contains the basic dispersion medium and the other ingredients from those enumerated above. In the case of sterile powders for the preparation of sterile injectable solutions, the preferred methods of preparation are vacuum-drying and freeze-drying techniques, which yield a powder of the active ingredient plus any additional desired ingredient from a previously sterile-filtered solution thereof.

The amount of rAAV particle or preparation and time of administration of such particle or preparation will be within the purview of the skilled artisan having benefit of the present teachings. It is likely, however, that the administration of therapeutically-effective amounts of the rAAV particles or preparations of the present disclosure may be achieved by a single administration, such as for example, a single injection of sufficient numbers of infectious particles to provide therapeutic benefit to the patient undergoing such treatment. Alternatively, in some circumstances, it may be desirable to provide multiple or successive administrations of the rAAV particle or preparation, either over a relatively short, or a relatively prolonged period of time, as may be determined by the medical practitioner overseeing the administration of such compositions.

If desired, rAAV particles may be administered in combination with other agents as well, such as, e.g., proteins or polypeptides or various pharmaceutically-active agents, including one or more administrations of therapeutic polypeptides, biologically active fragments, or variants thereof. In fact, there is virtually no limit to other components that may also be included, as long as the additional agents do not cause a significant adverse effect upon contact with the target cells or host tissues. The rAAV particles or preparations may thus be delivered along with various other pharmaceutically acceptable agents as required in the particular instance. Such compositions may be purified from host cells or other biological sources, or alternatively may be chemically synthesized as described herein.

In some embodiments, treatment of a subject with a rAAV particles as described herein achieves one, two, three, four, or more of the following effects, including, for example: (i) reduction or amelioration the severity of disease or symptom associated therewith; (ii) reduction in the duration of a symptom associated with a disease; (iii) protection against the progression of a disease or symptom associated therewith; (iv) regression of a disease or symptom associated therewith; (v) protection against the development or onset of a symptom associated with a disease; (vi) protection against the recurrence of a symptom associated with a disease; (vii) reduction in the hospitalization of a subject; (viii) reduction in the hospitalization length; (ix) an increase in the survival of a subject with a disease; (x) a reduction in the number of symptoms associated with a disease; (xi) an enhancement, improvement, supplementation, complementation, or augmentation of the prophylactic or therapeutic effect(s) of another therapy. In some embodiments, the disease or symptom is caused by hypertrophic cardiomyopathy or dilated cardiomyopathy. In some embodiments, the disease or symptom is dilated cardiomyopathy. In some embodiments, the disease or symptom is idiopathic dilated cardiomyopathy.

As is apparent to those skilled in the art in view of the teachings of this specification, an effective amount of viral vector to be added can be empirically determined. Administration can be administered in a single dose, a plurality of doses, continuously or intermittently throughout the course of treatment. Methods of determining the most effective means and dosages of administration are well known to those of skill in the art and will vary with the viral vector, the composition of the therapy, the target cells, and the subject being treated. Single and multiple administrations can be carried out with the dose level and pattern being selected by the treating physician.

### Kits

Herein are described compositions including one or more of the disclosed rAAV vectors comprised within a kit for diagnosing, preventing, treating or ameliorating one or more symptoms of a heart disease or condition, such as a cardiomyopathy. Such kits may be useful in the diagnosis, prophylaxis, and/or therapy or a human disease, and may be particularly useful in the treatment, prevention, and/or amelioration of one or more symptoms of heart disease, such as a cardiomyopathy. In some embodiments, the heart disease is caused by cardiomyopathy. In some embodiments, the heart disease is caused by hypertrophic cardiomyopathy or dilated cardiomyopathy. In some embodiments, the heart disease is dilated cardiomyopathy.

Kits comprising one or more of the disclosed rAAV vectors (as well as one or more virions, viral particles, transformed host cells or pharmaceutical compositions comprising such vectors); and instructions for using such kits in one or more therapeutic, diagnostic, and/or prophylactic clinical embodiments are also provided according to several embodiments. Such kits may comprise one or more reagents, restriction enzymes, peptides, therapeutics, pharmaceutical compounds, or means for delivery of the composition(s) to host cells, or to an animal (e.g., syringes, injectables, and the like). Depending on the embodiment, kits include those for treating, preventing, or ameliorating the symptoms of a disease, deficiency, dysfunction, and/or injury, or may include components for the large-scale production of the viral vectors themselves.

In some embodiments, a kit comprises one or more containers or receptacles comprising one or more doses of any of the described therapeutic. Such kits may be therapeutic in nature. In some embodiments, the kit contains a unit dosage, meaning a predetermined amount of a composition comprising, for example, a described therapeutic with or without one or more additional agents.

One or more of the components of a kit can be provided in one or more liquid or frozen solvents. The solvent can be aqueous or non-aqueous. The formulation in the kit can also be provided as dried powder(s) or in lyophilized form that can be reconstituted upon addition of an appropriate solvent.

In some embodiments, a kit comprises a label, marker, package insert, bar code and/or reader indicating directions of suitable usage of the kit contents. In some embodiments, the kit may comprise a label, marker, package insert, bar code and/or reader indicating that the kit contents may be administered in accordance with a certain dosage or dosing regimen to treat a subject.

In addition, a kit may also contain various reagents, including, but not limited to, wash reagents, elution reagents, and concentration reagents. Such reagents may be readily selected from among the reagents described herein, and from among conventional concentration reagents.

As used herein, the term “kit” may be used to describe variations of the portable, self-contained enclosure that includes at least one set of components to conduct one or more of the diagnostic or therapeutic methods of the invention.

### Combination Therapies

Multiple embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The compositions of the present disclosure may include rAAV particles or preparations, and/or rAAV vectors, either alone or in combination with one or more additional therapeutic agents, which may be obtained from natural or recombinant sources or chemically synthesized. In some embodiments, rAAV particles or preparations are administered in combination, either in the same composition or administered as part of the same treatment regimen, with a therapeutic agent containing a proteasome inhibitor, such as Bortezomib, or hydroxyurea.

If desired, rAAV particles may be administered in combination with other agents as well, such as, e.g., proteins or polypeptides or various pharmaceutically-active agents. This may, in some embodiments, reflect for example one or more administrations of therapeutic polypeptides, (e.g., a recombinant form of a functional peptide or protein that aids to replace or supplement the rAAV-based production of protein encoded by the transgene) biologically active fragments, or variants thereof. The rAAV particles or preparations may thus be delivered along with various other pharmaceutically acceptable agents as required in the particular instance. Such compositions may be purified from host cells or other biological sources, or alternatively may be chemically synthesized as described herein.

The amount of compositions containing the disclosed rAAV particles and additional therapeutic agent, and the time of administration of such compositions, will be within the purview of the skilled artisan having benefit of the present teachings. It is likely, however, that the administration of therapeutically-effective amounts of the compositions of the present disclosure may be achieved by co-administration or separate administration. The disclosed rAAV particles and/or rAAV vectors may be delivered before, after, or simultaneously with any of the disclosed additional therapeutic agents. In some embodiments, the rAAV particle is delivered before the additional therapeutic agent. In some embodiments, the rAAV particle is delivered after the additional therapeutic agent.

In some embodiments, the additional therapeutic agent comprises an anti-inflammatory agent. The anti-inflammatory agent can be, but is not limited to, a corticosteroid, cortisone hydrocortisone, hydrocortisone-21-monoesters (e.g., hydrocortisone-21-acetate, hydrocortisone-21-butyrate, hydrocortisone-21-propionate, hydrocortisone-21-valerate, etc.), hydrocortisone-17,21-diesters (e.g., hydrocortisone-17,21-diacetate, hydrocortisone-17-acetate-21-butyrate, hydrocortisone-17,21-dibutyrate, etc.), alclometasone, dexamethasone, flumethasone, prednisolone, methylprednisolone, betamethasone, typically as betamethasone benzoate or betamethasone diproprionate; fluocinonide; prednisone; and triamcinolone, typically as triamcinolone acetonide. In some embodiments, the anti-inflammatory agent is a mast cell degranulation inhibitor, such as, without limitation, cromolyn (5,5′-(2-hydroxypropane-1,3-diyl)bis(oxy)bis(4-oxo-4H-chromene-2-carboxylic acid) (also known as cromoglycate), and 2-carboxylatochromon-5′-yl-2-hydroxypropane derivatives such as bis(acetoxymethyl), disodium cromoglycate, nedocromil (9-ethyl-4,6-dioxo-10-propyl-6,9-dihydro-4H-pyrano[3,2-g]quinoline-2,8-dicarboxylic acid) and tranilast (2-{[(2E)-3-(3,4-dimethoxyphenyl) prop-2-enoyl]amino}), and lodoxamide (2-[2-chloro-5-cyano-3-(oxaloamino) anilino]-2-oxoacetic acid). In some embodiments, the anti-inflammatory agent is a nonsteroidal anti-inflammatory drugs (NSAIDs), such as, without limitation, aspirin compounds (acetylsalicylates), non-aspirin salicylates, diclofenac, diflunisal, etodolac, fenoprofen, flurbiprofen, ibuprofen, indomethacin, ketoprofen, meclofenamate, naproxen, naproxen sodium, phenylbutazone, sulindac, and tometin.

In some embodiments, the anti-inflammatory agent comprises an antihistamine. The antihistamine can be, but is not limited to, clemastine, clemastine fumarate (2(R)-[2-[1-(4-Chlorophenyl)-1-phenyl-ethoxy]ethyl-1-methylpyrrolidine), dexmedetomidine, doxylamine, loratidine, desloratidine and promethazine, and diphenhydramine, or pharmaceutically acceptable salts, solvates or esters thereof. In some embodiments, the antihistamine includes, without limitation, azatadine, azelastine, burfroline, cetirizine, cyproheptadine, doxantrozole, etodroxizine, forskolin, hydroxyzine, ketotifen, oxatomide, pizotifen, proxicromil, N,N′-substituted piperazines or terfenadine. In some embodiments, the antihistamine is an H1 antagonist, such as, but not limited to, cetirizine, chlorpheniramine, dimenhydrinate, diphenhydramine, fexofenadine, hydroxyzine, orphenadrine, pheniramine, and doxylamine. In some embodiments, the antihistamine is an H2 antagonist, such as, but not limited to, cimetidine, famotidine, lafutidine, nizatidine, ranitidine, and roxatidine.

In some embodiments, the additional therapeutic agent comprises an antiviral agent, including antiretroviral agents. Suitable antiviral agents include, without limitation, remdesivir, acyclovir, famcyclovir, ganciclovir, foscarnet, idoxuridine, sorivudine, trifluorothymidine, valacyclovir, vidarabine, didanosine, dideoxyinosine, stavudine, zalcitabine, zidovudine, amantadine, interferon alpha, ribavirin and rimantadine.

In some embodiments, the additional therapeutic agent comprises an antibiotic. Non-limiting examples of suitable antibiotics include beta-lactams such as penicillins, aminopenicillins (e.g., amoxicillin, ampicillin, hetacillin, etc.), penicillinase resistant antibiotics (e.g., cloxacillin, dicloxacillin, methicillin, nafcillin, oxacillin, etc.), extended spectrum antibiotics (e.g., axlocillin, carbenicillin, mezlocillin, piperacillin, ticarcillin, etc.); cephalosporins (e.g., cefadroxil, cefazolin, cephalixin, cephalothin, cephapirin, cephradine, cefaclor, cefacmandole, cefmetazole, cefonicid, ceforanide, cefotetan, cefoxitin, cefprozil, cefuroxime, loracarbef, cefixime, cefoperazone, cefotaxime, cefpodoxime, ceftazidime, ceftiofur, ceftizoxime, ceftriaxone, moxalactam, etc.); monobactams such as aztreonam; Carbapenems such as imipenem and eropenem; quinolones (e.g., ciprofloxacin, enrofloxacin, difloxacin, orbifloxacin, marbofloxacin, etc.); chloramphenicols (e.g., chloramphenicol, thiamphenicol, florfenicol, etc.); tetracyclines (e.g., chlortetracycline, tetracycline, oxytetracycline, doxycycline, minocycline, etc.); macrolides (e.g., erythromycin, tylosin, tlimicosin, clarithromycin, azithromycin, etc.); lincosamides (e.g., lincomycin, clindamycin, etc.); aminoglycosides (e.g., gentamicin, amikacin, kanamycin, apramycin, tobramycin, neomycin, dihydrostreptomycin, paromomycin, etc.); sulfonamides (e.g., sulfadmethoxine, sfulfamethazine, sulfaquinoxaline, sulfamerazine, sulfathiazole, sulfasalazine, sulfadiazine, sulfabromomethazine, suflaethoxypyridazine, etc.); glycopeptides (e.g., vancomycin, teicoplanin, ramoplanin, and decaplanin; and other antibiotics (e.g., rifampin, nitrofuran, virginiamycin, polymyxins, tobramycin, etc.)).

In some embodiments, the additional therapeutic agent comprises an antifungal agent, such as, but not limited to, itraconazole, ketoconazole, fluoconazole, and amphotericin B. In some embodiments, the therapeutic agent is an antiparasitic agents, such as, but not limited to, the broad spectrum antiparasitic medicament nitazoxanide; antimalarial drugs and other antiprotozoal agents (e.g., artemisins, mefloquine, lumefantrine, tinidazole, and miltefosine); anthelminthics such as mebendazole, thiabendazole, and ivermectin; and antiamoebic agents such as rifampin and amphotericin B.

In some embodiments, the additional therapeutic agent comprises an analgesic agent, including, without limitation, opioid analgesics such as alfentanil, buprenorphine, butorphanol, codeine, drocode, fentanyl, hydrocodone, hydromorphone, levorphanol, meperidine, methadone, morphine, nalbuphine, oxycodone, oxymorphone, pentazocine, propoxyphene, sufentanil, and tramadol; and nonopioid analgesics such as apazone, etodolac, diphenpyramide, indomethacin, meclofenamate, mefenamic acid, oxaprozin, phenylbutazone, piroxicam, and tolmetin.

The disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. In addition, where features or aspects of the disclosure are described in terms of Markush groups, those skilled in the art will recognize that the disclosure is also thereby described in terms of any individual member or subgroup of members of the Markush group.

Any titles or subheadings used herein are for organization purposes and should not be used to limit the scope of embodiments disclosed herein.

### EXAMPLES

The following examples are illustrative only and are not intended to be a limitation on the scope of the invention.

### Materials and Methods

### Construct Design.

RBM20 cDNA was codon optimized for expression in human tissues and was subcloned into a plasmid backbone suitable for production of AAV. The constructs were engineered to comprise the elements as provided in Tables 1 and 2 below. Schematic representations of the constructs are provided in FIGS. 1 and 2. The chimeric intron sequence harbors a unique FseI restriction site which, in cases where it is desirable to reduce or eliminate expression of mutant RBM20, is used for subcloning knockdown cassettes (e.g., shRNA expression cassettes) into the construct.

TABLE 5
Construct 1 (pTR-TNNT2-RBM20; FIG. 1)

| | Nucleotide (Nt) | Amino acid (AA) |
|---|---|---|
| Elements (5′ −> 3′) | sequence | sequence (as applicable) |
| 5′ ITR (ITR-L) | TTGGCCACTCCCTCT | |
| | CTGCGCGCTCGCTCG | |
| | CTCACTGAGGCCGG | |
| | GCGACCAAAGGTCG | |
| | CCCGACGCCCGGGCT | |
| | TTGCCCGGGCGGCCT | |
| | CAGTGAGCGAGCGA | |
| | GCGCGCAGAGAGGG | |
| | AGTGGCCAACTCCAT | |
| | CACTAGGGGTTCCT | |
| TNNT2 promoter | GTCATGGAGAAGAC | |
| | CCACCTTGCAGATGT | |
| | CCTCACTGGGGCTGG | |
| | CAGAGCCGGCAACC | |
| | TGCCTAAGGCTGCTC | |
| | AGTCCATTAGGAGCC | |
| | AGTAGCCTGGAAGA | |
| | TGTCTTTACCCCCAG | |
| | CATCAGTTCAAGTGG | |
| | AGCAGCACATAACT | |
| | CTTGCCCTCTGCCTT | |
| | CCAAGATTCTGGTGC | |
| | TGAGACTTATGGAGT | |
| | GTCTTGGAGGTTGCC | |
| | TTCTGCCCCCCAACC | |
| | CTGCTCCCAGCTGGC | |
| | CCTCCCAGGCCTGGG | |
| | TTGCTGGCCTCTGCT | |
| | TTATCAGGATTCTCA | |
| | AGAGGGACAGCTGG | |
| | TTTATGTTGCATGAC | |
| | TGTTCCCTGCATATC | |
| | TGCTCTGGTTTTAAA | |
| | TAGCTTATCTGAGCA | |
| | GCTGGAGGACCACA | |
| | TGGGCTTATATGGCG | |
| | TGGGGTACATGATCC | |
| | TGTAGCCTTGTCCCT | |
| | GGCACCTGCCAAAA | |
| | TAGCAGCCAACACC | |
| | CCCCACCCCCACCGC | |
| | CATCCCCCTGCCCCA | |
| | CCCGTCCCCTGTCGC | |
| | ACATTCCTCCCTCCG | |
| | CAGGGCTGGCTCACC | |
| | AGGCCCCAGCCCAC | |
| | ATGCCTGCTTAAAGC | |
| | CCTCTCCATCCTCTG | |
| | CCTCACCCAGTCCCC | |
| | GCTGAGACTGAGCA | |
| | GACGCCTCCA | |
| chimeric intron (with FseI site in bold | CAGGTAAGTATCAA | |
| underline) | GGTTACAAGACAGG | |
| | TTTAAGGAGACCAAT | |
| | AGAAACTGGGCTTGT | |
| | CGAGACAGAGGGCC | |
| | GGCCAAGACTCTTG | |
| | CGTTTCTGATAGGCA | |
| | CCTATTGGTCTTACT | |
| | GACATCCACTTTGCC | |
| | TTTCTCTCCACAGGG | |
| | T | |
| ACC65I endonuclease site | GGTACC | |
| RBM20 cDNA | ATGGTGCTGGCAGC | MVLAAAMSQDADPS |
| | AGCCATGAGCCAGG | GPEQPDRVACSVPGA |
| | ACGCGGACCCCAGC | RASPAPSGPRGMQQP |
| | GGTCCGGAGCAGCC | PPPPQPPPPPQAGLPQI |
| | GGACAGAGTTGCCT | IQNAAKLLDKNPFSVS |
| | GCAGTGTGCCTGGTG | NPNPLLPSPASLQLAQ |
| | CCCGGGCGTCCCCGG | LQAQLTLHRLKLAQT |
| | CACCCTCCGGCCCGC | AVTNNTAAATVLNQ |
| | GAGGGATGCAGCAG | VLSKVAMSQPLFNQL |
| | CCGCCGCCGCCGCCC | RHPSVITGPHGHAGV |
| | CAGCCACCGCCCCCG | PQHAAAIPSTRFPSNA |
| | CCCCAAGCCGGCCTA | IAFSPPSQTRGPGPSM |
| | CCCCAGATCATCCAA | NLPNQPPSAMVMHPF |
| | AATGCCGCCAAGCTC | TGVMPQTPGQPAVIL |
| | CTGGACAAGAACCC | GIGKTGPAPATAGFY |
| | ATTCTCGGTCAGTAA | EYGKASSGQTYGPET |
| | CCCGAACCCTCTGCT | DGQPGFLPSSASTSGS |
| | TCCTTCACCTGCCAG | VTYEGHYSHTGQDG |
| | TCTCCAGCTGGCTCA | QAAFSKDFYGPNSQG |
| | ACTGCAGGCCCAGCT | SHVASGFPAEQAGGL |
| | CACCCTCCACCGGCT | KSEVGPLLQGTNSQW |
| | GAAGCTGGCACAGA | ESPHGFSGQSKPDLTA |
| | CAGCTGTCACCAACA | GPMWPPPHNQPYELY |
| | ACACTGCAGCCGCC | DPEEPTSDRTPPSFGG |
| | ACAGTCCTGAACCA | RLNNSKQGFLIGAGRR |
| | AGTCCTCTCCAAAGT | AKEDQALLSVRPLQA |
| | GGCCATGTCCCAGCC | HELNDFHGVAPLHLP |
| | TCTCTTCAATCAACT | IICSICDKKVFDLKD |
| | GAGGCATCCGTCTGT | WELHVKGKLHAQKC |
| | GATCCACTGGCCCCCA | LVFSENAGIRCILGSA |
| | CGGCCATGCTGGGGT | EGTLCASPNSTAVYN |
| | TCCCCAACATGCTGC | PAGNEDYASNLGTSY |
| | AGCCATACCCAGTAC | VPIPARSFTQSSPTFPL |
| | CCGGTTTCCCTCTAA | ASVGTFFAQRKGAGR |
| | TGCAATTGCCTTTTC | VVHICNLPEGSCTEND |
| | ACCCCCCAGCCAGA | VINLGLPFGKVTNYIL |
| | CACGAGGCCCCGGA | MKSTNQAFLEMAYTE |
| | CCCTCCATGAACCTT | AAQAMVQYYQEKSA |
| | CCCAACCAGCCACCC | VINGEKLLIRMSKRY |
| | AGTGCCATGGTGATG | KELQLKKPGKAVAAII |
| | CATCCTTTCACTGGG | QDIHSQRERDMFREA |
| | GTAATGCCTCAGACC | DRYGPERPRSRSPVSR |
| | CCTGGCCAGCCAGC | SLSPRSHTPSFTSCSSS |
| | AGTCATCTTGGGCAT | HSPPGPSRADWGNGR |
| | TGGCAAGACTGGGC | DSWEHSPYARREEER |
| | CTGCTCCAGCTACAG | DPAPWRDNGDDKRD |
| | CAGGATTCTATAGT | RMDPWAHDRKHHPR |
| | ATGGCAAAGCCAGC | QLDKAELDERPEGGR |
| | TCTGGCCAGACATAT | PHREEKYPRSGSPNLPH |
| | GGCCCTGAAACAGA | SVSSULSREDGYYRK |
| | TGGTCAGCCTGGCTT | EPKAKWDKYLKQQQ |
| | CCTGCCATCCTCGGC | DAPGRSRRKDEARLR |
| | CTCAACCTCGGGCAG | ESRHPHPDDSGKEDG |
| | TGTGACCTATGAAGG | LGPKVTRAPEGAKAK |
| | GCACTACAGCCACA | QNEKNKTKRTDRDQE |
| | CAGGGCAGGATGGT | GADDRKENTMAENE |
| | CAAGCTGCCTTTTCC | AGKEEQEGMEESPQS |
| | AAAGATTTTTACGGA | VGRQEKEAEFSDPEN |
| | CCCAACTCCCAAGGT | TRTKKEQDWESESEA |
| | TCACATGTGGCCAGC | EGESWYPTNMEELVT |
| | GGATTTCCAGCTGAG | VDEVGEEEDFIVEPDI |
| | CAGGCTGGGGGCCT | PELEEIVPIDQKDKICP |
| | GAAAAGTGAGGTCG | ETCLCVTTTLDLDLA |
| | GGCCACTGCTGCAG | QDFPKEGVKAVGNG |
| | GGCACAAACAGCCA | AAEISLKSPRELPSAST |
| | ATGGGAGAGCCCCC | SCPSDMDVEMPGLNL |
| | ATGGATTCTCGGGCC | DAERKPAESETGLSLE |
| | AAAGCAAGCCTGAT | DSDCYEKEAKGVESS |
| | CTCACAGCAGGTCCC | DVHPAPTVQQMSSPK |
| | ATGTGGCCTCCACCC | PAEERARQPSPFVDD |
| | CACAACCAGCCCTAT | CKTRGTPEDGACEGS |
| | CAGCTGTACGACCCC | PLEEKASPPIETDLQN |
| | GAGGAACCAACCTC | QACQEVLTPENSRYV |
| | AGACAGGACACCTC | EMKQPLSLPSWEPEDV |
| | CTTCCTTCGGGGGTC | ELKQPLSLPSWEPEDV |
| | GGCTTAACAACAGC | FSELSIPLGVEFVVPRT |
| | AAACAGGGTTTTATC | GFYCKLCGLFYTSEET |
| | GGTGCTGGGCGGAG | AKMSHCRSAVHYRN |
| | GGCCAAGGAGGACC | LQKYLSQLAEEGLKE |
| | AACGTTGCTATCTG | TEGADSPRPEDSGVIP |
| | TGCGGCCTCTGCAGG | RFERKKL |
| | CTCATGAGCTGAACG | |
| | ACTTTCACGGTGTGG | |
| | CCCCCCTCCACTTGC | |
| | CGCATATCTGTAGCA | |
| | TCTGTGACAAGAAG | |
| | GTGTTTGATTTGAAG | |
| | GACTGGGAGCTGCA | |
| | TGTGAAAGGGAAGC | |
| | TGCACGCTCAGAAAT | |
| | GCCTGGTCTTCTCTG | |
| | AAAATGCTGGCATCC | |
| | GGTGTATACTTGGTT | |
| | CGGCAGAGGGAACA | |
| | TTGTGTGCTTCTCCC | |
| | AACAGCACAGCTGTT | |
| | TATAACCCTGCTGGG | |
| | AATGAAGATTATGCC | |
| | TCAAATCTTGGAACA | |
| | TCATACGTGCCCATT | |
| | CCAGCAAGGTCATTC | |
| | ACTCAGTCAAGCCCC | |
| | ACATTTCCTTTGGCT | |
| | TCTGTGGGGACAACT | |
| | TTTGCACAGCGGAA | |
| | AGGGGCTGGCCGTG | |
| | TGGTGCACATCTGCA | |
| | ATCTCCCTGAAGGAA | |
| | GCTGCACTGAGAAT | |
| | GACGTCATTAACCTG | |
| | GGGCTGCCCTTTGGA | |
| | AAGGTCACTAATTAC | |
| | ATCCTCATGAAATCG | |
| | ACTAATCAGGCCTTT | |
| | TTAGAGATGGCTTAC | |
| | ACAGAAGCTGCACA | |
| | GGCCATGGTCCAGTA | |
| | TTATCAAGAAAAATC | |
| | TGCTGTGATCAATGG | |
| | TGAGAAGTTGCTCAT | |
| | TCGGATGTCCAAGA | |
| | GATACAAGGAATTG | |
| | CAGCTCAAGAAACC | |
| | CGGGAAGGCCGTGG | |
| | CTGCCATCATCCAGG | |
| | ACATCCATTCCCAGA | |
| | GGGAGAGGGACATG | |
| | TTCCGGGAAGCAGA | |
| | CAGATATGGCCCAG | |
| | AAAGGCCGCGGTCT | |
| | CGTAGTCCGGTGAGC | |
| | CGGTCACTCTCCCCG | |
| | AGGTCCCACACTCCC | |
| | AGCTTCACCTCCTGC | |
| | AGCTCTTCCCACAGC | |
| | CCTCCGGGCCCCTCC | |
| | CGGGCTGACTGGGG | |
| | CAATGGCCGGGACT | |
| | CCTGGGAGCACTCTC | |
| | CCTATGCCAGGAGG | |
| | GAGGAAGAGCGAGA | |
| | CCCGGCTCCCTGGAG | |
| | GGACAACGGAGATG | |
| | ACAAGAGGGACAGG | |
| | ATGGACCCCTGGGC | |
| | ACATGATCGCAAAC | |
| | ACCACCCCCGGCAA | |
| | CTGGACAAGGCTGA | |
| | GTTGGACGAGCGAC | |
| | CAGAAGGAGGGAGG | |
| | CCCCACCGGGAGAA | |
| | GTACCCGAGATCTGG | |
| | GTCTCCCAACCTGCC | |
| | CCACTCTGTGTCCAG | |
| | CTACAAAAGCCGTG | |
| | AAGACGGCTACTAC | |
| | CGGAAAGAGCCCAA | |
| | AGCCAAGTGGGACA | |
| | AGTATCTGAAGCAG | |
| | CAGCAGGATGCCCC | |
| | CGGGAGGTCCAGGA | |
| | GGAAAGACGAGGCC | |
| | AGGCTGCGGGAAAG | |
| | CAGACACCCCCATCC | |
| | GGATGACTCAGGCA | |
| | AGGAAGATGGGCTG | |
| | GGGCCAAAGGTCAC | |
| | TAGGGCCCCTGAGG | |
| | GCGCCAAGGCCAAG | |
| | CAGAATGAGAAAAA | |
| | TAAAACCAAGAGAA | |
| | CTGATAGAGACCAA | |
| | GAAGGAGCTGATGA | |
| | TAGAAAAGAAAACA | |
| | CAATGGCAGAGAAT | |
| | GAGGCTGGAAAAGA | |
| | GGAACAGGAGGGCA | |
| | TGGAAGAAAGCCCT | |
| | CAATCAGTGGGCAG | |
| | ACAGGAGAAAGAAG | |
| | CAGAGTTCTCTGATC | |
| | CGGAAAACACAAGG | |
| | ACAAAGAAGGAACA | |
| | AGATTGGGAGAGTG | |
| | AAAGTGAGGCAGAG | |
| | GGGGAGAGCTGGTA | |
| | TCCCACTAACATGGA | |
| | GGAGCTGGTGACAG | |
| | TGGACGAGGTTGGG | |
| | GAAGAAGAAGATTT | |
| | TATCGTGGAACCAG | |
| | ACATCCCAGAGCTG | |
| | GAAGAAATTGTGCC | |
| | CATTGACCAGAAAG | |
| | ACAAAATTTGCCCAG | |
| | AAACATGTCTGTGTG | |
| | TGACAACCACCTTAG | |
| | ACTTAGACCTGGCCC | |
| | AGGATTTCCCCAAGG | |
| | AAGGAGTCAAGGCC | |
| | GTAGGGAATGGGGC | |
| | TGCAGAAATCAGCCT | |
| | CAAGTCACCCAGAG | |
| | AACTGCCCTCTGCTT | |
| | CCACAAGCTGTCCCA | |
| | GTGACATGGACGTG | |
| | GAAATGCCTGGCCTA | |
| | AATCTGGATGCTGAG | |
| | CGGAAGCCAGCTGA | |
| | AAGTGAGACAGGCC | |
| | TCTCCCTGGAGGATT | |
| | CAGATTGCTACGAG | |
| | AAGGAGGCAAAGGG | |
| | AGTGGAGAGCTCAG | |
| | ATGTTCATCCAGCCC | |
| | CTACAGTCCAGCAA | |
| | ATGTCTTCCCCTAAG | |
| | CCAGCAGAGGAGAG | |
| | GGCCCGGCAGCCAA | |
| | GCCCATTTGTGGATG | |
| | ATTGCAAGACCAGG | |
| | GGGACCCCCGAAGA | |
| | TGGGGCTTGTGAAG | |
| | GCAGCCCCCTGGAG | |
| | GAGAAAGCCAGCCC | |
| | CCCCATCGAAACTGA | |
| | CCTCCAAAACCAAG | |
| | CCTGCCAAGAAGTGT | |
| | TGACCCCGGAAAAC | |
| | TCCAGGTACGTGGA | |
| | AATGAAATCTCTGGA | |
| | GGTGAGGTCACCAG | |
| | AGTACACTGAAGTG | |
| | GAACTGAAACAGCC | |
| | CCTTTCTTTGCCCTCT | |
| | TGGGAACCAGAGGA | |
| | TGTGTTCAGTGAACT | |
| | TAGCATTCCTCTAGG | |
| | GGTGGAGTTCGTGGT | |
| | TCCCAGGACTGGCTT | |
| | TTATTGCAAGCTGTG | |
| | TGGGCTGTTCTACAC | |
| | GAGCGAGGAGACAG | |
| | CAAAGATGAGCCAC | |
| | TGCCGCAGCGCTGTC | |
| | CACTACAGGAACTTA | |
| | CAGAAATATTTGTCC | |
| | CAGCTGGCCGAGGA | |
| | GGGCCTCAAGGAGA | |
| | CCGAGGGGGCAGAT | |
| | AGCCCGAGGCCAGA | |
| | GGACAGCGGAATCG | |
| | TGCCACGCTTCGAAA | |
| | GGAAAAAGCTCTGA | |
| polyA | AATAAAAGATCCTTA | |
| | TTTTCATTGGATCTG | |
| | TGTGTTGGTTTTTTG | |
| | TGTG | |
| 5′ ITR (ITR-R) | AGGAACCCCTAGTG | |
| | ATGGAGTTGGCCACT | |
| | CCCTCTCTGCGCGCT | |
| | CGCTCGCTCACTGAG | |
| | GCCGGGCGACCAAA | |
| | GGTCGCCCGACGCCC | |
| | GGGCTTTGCCCGGGC | |
| | GGCCTCAGTGAGCG | |
| | AGCGAGCGCGCAGA | |
| | GAGGGAGTGGCCAA | |

TABLE 6
Construct 2 (pTR2-MHCK9-RBM20; FIG. 2)

| | | Amino acid (AA) sequence |
|---|---|---|
| Elements (5′ −> 3′) | Nt sequence | (as applicable) |
| ITR-L | TTGGCCACTCCCTCTCTGCG | |
| | CGCTCGCTCGCTCACTGAG | |
| | GCCGGGCGACCAAAGGTCG | |
| | CCCGACGCCCGGGCTTTGC | |
| | CCGGGCGGCCTCAGTGAGC | |
| | GAGCGAGCGCGCAGAGAG | |
| | GGAGTGGCCAACTCCATCA | |
| | CTAGGGGTTCCT | |
| Alpha MHC Enhancer | ACCCTTCAGATTAAAAATA | |
| | ACTGAGGTAAGGGCCTGGG | |
| | TAGGGGAGGTGGTGTGAGA | |
| | CGCTCCTGTCTCTCCTCTAT | |
| | CTGCCCATCGGCCCTTTGG | |
| | GGAGGAGGAATGTGCCCAA | |
| | GGACTAAAAAAAGGCCATG | |
| | GAGCCAGAGGGGCGAGGG | |
| | CAACAGACCTTTCATGGGC | |
| | AAACCTTGGGGCCCTGCTG | |
| | T | |
| MHCK9 Enhancer | CTGCCCATGTAAGGAGGCA | |
| | AGGCCTGGGGACACCCGAG | |
| | ATGCCTGGTTATAATTAAC | |
| | CCAGACATGTGGCTGCCCC | |
| | CCCCCCCCCAACACCTGCT | |
| | GCCTCTAAAAATAACC | |
| MHCK9 Promoter | GTTCCCGGCGAAGGGCCAG | |
| | CTGTCCCCCGCCAGCTAGA | |
| | CTCAGCACTTAGTTTAGGA | |
| | ACCAGTGAGCAAGTCAGCC | |
| | CTTGGGGCAGCCCATACAA | |
| | GGCCATGGGGCTGGGCAAG | |
| | CTGCACGCCTGGGTCCGGG | |
| | GTGGGCACGGTGCCCGGGC | |
| | AACGAGCTGAAAGCTCATC | |
| | TGCTCTCAGGGGCCCCTCC | |
| | CTGGGGACAGCCCCTCCTG | |
| | GCTAGTCACACCCTGTAGG | |
| | CTCCTCTATATAACCCAGG | |
| | GGCACAGGGGCTGCCCTC | |
| MHCK9 5′ UTR | ACCACCACCTCCACAGCAC | |
| | AGACAGACACTCAGGAGCA | |
| | GCCAG | |
| chimeric intron (with Fsel site in | CAGGTAAGTATCAAGGTTA | |
| bold underline) | CAAGACAGGTTTAAGGAGA | |
| | CCAATAGAAACTGGGCTTG | |
| | TCGAGACAGAGGGCCGGC | |
| | CAAGACTCTTGCGTTTCTG | |
| | ATAGGCACCTATTGGTCTT | |
| | ACTGACATCCACTTTGCCTT | |
| | TCTCTCCACAGGGT | |
| RBM20 | ATGGTGCTGGCAGCAGCCA | MVLAAAMSQDADPSGPEQP |
| | TGAGCCAGGACGCGGACCC | DRVACSVPGARASPAPSGPR |
| | CAGCGGTCCGGAGCAGCCG | GMQQPPPPPQPPPPPQAGLPQ |
| | GACAGAGTTGCCTGCAGTG | IIQNAAKLLDKNPFSVSNPNP |
| | TGCCTGGTGCCCGGGCGTC | LLPSPASLQLAQLQAQLTLH |
| | CCCGGCACCCTCCGGCCCG | RLKLAQTAVINNTAAATVL |
| | CGAGGGATGCAGCAGCCGC | NQVLSKVAMSQPLFNQLRHP |
| | CGCCGCCGCCCCAGCCACC | SVITGPHGHAGVPQHAAAIP |
| | GCCCCCGCCCCAAGCCGGC | STRFPSNAIAFSPPSQTRGPGP |
| | CTACCCCAGATCATCCAAA | SMNLPNQPPSAMVMHPFTG |
| | ATGCCGCCAAGCTCCTGGA | VMPQTPGQPAVILGIGKTGP |
| | CAAGAACCCATTCTCGGTC | APATAGFYEYGKASSGQTY |
| | AGTAACCCGAACCCTCTGC | GPETDGQPGFLPSSASTSGSV |
| | TTCCTTCACCTGCCAGTCTC | TYEGHYSHTGQDGQAAFSK |
| | CAGCTGGCTCAACTGCAGG | DFYGPNSQGSHVASGFPAEQ |
| | CCCAGCTCACCCTCCACCG | AGGLKSEVGPLLQGTNSQW |
| | GCTGAAGCTGGCACAGACA | ESPHGFSGQSKPDLTAGPMW |
| | GCTGTCACCAACAACACTG | PPPHNQPYELYDPEEPTSDRT |
| | CAGCCGCCACAGTCCTGAA | PPSFGGRLNNSKQGFIGAGR |
| | CCAAGTCCTCTCCAAAGTG | RAKEDQALLSVRPLQAHELN |
| | GCCATGTCCCAGCCTCTCTT | DFHGVAPLHLPHICSICDKKV |
| | CAATCAACTGAGGCATCCG | FDLKDWELHVKGKLHAQKC |
| | TCTGTGATCACTGGCCCCC | LVFSENAGIRCILGSAEGTLC |
| | ACGGCCATGCTGGGGTTCC | ASPNSTAVYNPAGNEDYASN |
| | CCAACATGCTGCAGCCATA | LGTSYVPIPARSFTQSSPTFPL |
| | CCCAGTACCCGGTTTCCCTC | ASVGTTFAQRKGAGRVVHIC |
| | TAATGCAATTGCCTTTTCAC | NLPEGSCTENDVINLGLPFGK |
| | CCCCCAGCCAGACACGAGG | VTNYILMKSTNQAFLEMAYT |
| | CCCCGGACCCTCCATGAAC | EAAQAMVQYYQEKSAVING |
| | CTTCCCAACCAGCCACCCA | EKLLIRMSKRYKELQLKKPG |
| | GTGCCATGGTGATGCATCC | KAVAAIIQDIHSQRERDMFR |
| | TTTCACTGGGGTAATGCCTC | EADRYGPERPRSRSPVSRSLS |
| | AGACCCCTGGCCAGCCAGC | PRSHTPSFTSCSSSHSPPGPSR |
| | AGTCATCTTGGGCATTGGC | ADWGNGRDSWEHSPYARRE |
| | AAGACTGGGCCTGCTCCAG | EERDPAPWRDNGDDKRDRM |
| | CTACAGCAGGATTCTATGA | DPWAHDRKHHPRQLDKAEL |
| | GTATGGCAAAGCCAGCTCT | DERPEGGRPHREKYPRSGSP |
| | GGCCAGACATATGGCCCTG | NLPHSVSSYKSREDGYYRKE |
| | AAACAGATGGTCAGCCTGG | PKAKWDKYLKQQQDAPGRS |
| | CTTCCTGCCATCCTCGGCCT | RRKDEARLRESRHPHPDDSG |
| | CAACCTCGGGCAGTGTGAC | KEDGLGPKVTRAPEGAKAK |
| | CTATGAAGGGCACTACAGC | QNEKNKTKRTDRDQEGADD |
| | CACACAGGGCAGGATGGTC | RKENTMAENEAGKEEQEGM |
| | AAGCTGCCTTTTCCAAAGA | EESPQSVGRQEKEAEFSDPE |
| | TTTTTACGGACCCAACTCCC | NTRTKKEQDWESESEAEGES |
| | AAGGTTCACATGTGGCCAG | WYPTNMEELVTVDEVGEEE |
| | CGGATTTCCAGCTGAGCAG | DFIVEPDIPELEEIVPIDQKDK |
| | GCTGGGGGCCTGAAAAGTG | ICPETCLCVTTTLDLDLAQDF |
| | AGGTCGGGCCACTGCTGCA | PKEGVKAVGNGAAEISLKSP |
| | GGGCACAAACAGCCAATGG | RELPSASTSCPSDMDVEMPG |
| | GAGAGCCCCCATGGATTCT | LNLDAERKPAESETGLSLED |
| | CGGGCCAAAGCAAGCCTGA | SDCYEKEAKGVESSDVHPAP |
| | TCTCACAGCAGGTCCCATG | TVQQMSSPKPAEERARQPSP |
| | TGGCCTCCACCCCACAACC | FVDDCKTRGTPEDGACEGSP |
| | AGCCCTATGAGCTGTACGA | LEEKASPPIETDLQNQACQE |
| | CCCCGAGGAACCAACCTCA | VLTPENSRYVEMKSLEVRSP |
| | GACAGGACACCTCCTTCCT | EYTEVELKQPLSLPSWEPED |
| | TCGGGGGTCGGCTTAACAA | VESELSIPLGVEFVVPRTGFY |
| | CAGCAAACAGGGTTTTATC | CKLCGLFYTSEETAKMSHCR |
| | GGTGCTGGGCGGAGGGCCA | SAVHYRNLQKYLSQLAEEGL |
| | AGGAGGACCAGGCGTTGCT | KETEGADSPRPEDSGIVPRFE |
| | ATCTGTGCGGCCTCTGCAG | RKKL |
| | GCTCATGAGCTGAACGACT | |
| | TTCACGGTGTGGCCCCCCTC | |
| | CACTTGCCGCATATCTGTA | |
| | GCATCTGTGACAAGAAGGT | |
| | GTTTGATTTGAAGGACTGG | |
| | GAGCTGCATGTGAAAGGGA | |
| | AGCTGCACGCTCAGAAATG | |
| | CCTGGTCTTCTCTGAAAATG | |
| | CTGGCATCCGGTGTATACTT | |
| | GGTTCGGCAGAGGGAACAT | |
| | TGTGTGCTTCTCCCAACAGC | |
| | ACAGCTGTTTATAACCCTG | |
| | CTGGGAATGAAGATTATGC | |
| | CTCAAATCTTGGAACATCA | |
| | TACGTGCCCATTCCAGCAA | |
| | GGTCATTCACTCAGTCAAG | |
| | CCCCACATTTCCTTTGGCTT | |
| | CTGTGGGGACAACTTTTGC | |
| | ACAGCGGAAAGGGGCTGGC | |
| | CGTGTGGTGCACATCTGCA | |
| | ATCTCCCTGAAGGAAGCTG | |
| | CACTGAGAATGACGTCATT | |
| | AACCTGGGGCTGCCCTTTG | |
| | GAAAGGTCACTAATTACAT | |
| | CCTCATGAAATCGACTAAT | |
| | CAGGCCTTTTTAGAGATGG | |
| | CTTACACAGAAGCTGCACA | |
| | GGCCATGGTCCAGTATTAT | |
| | CAAGAAAAATCTGCTGTGA | |
| | TCAATGGTGAGAAGTTGCT | |
| | CATTCGGATGTCCAAGAGA | |
| | TACAAGGAATTGCAGCTCA | |
| | AGAAACCCGGGAAGGCCGT | |
| | GGCTGCCATCATCCAGGAC | |
| | ATCCATTCCCAGAGGGAGA | |
| | GGGACATGTTCCGGGAAGC | |
| | AGACAGATATGGCCCAGAA | |
| | AGGCCGCGGTCTCGTAGTC | |
| | CGGTGAGCCGGTCACTCTC | |
| | CCCGAGGTCCCACACTCCC | |
| | AGCTTCACCTCCTGCAGCTC | |
| | TTCCCACAGCCCTCCGGGC | |
| | CCCTCCCGGGCTGACTGGG | |
| | GCAATGGCCGGGACTCCTG | |
| | GGAGCACTCTCCCTATGCC | |
| | AGGAGGGAGGAAGAGCGA | |
| | GACCCGGCTCCCTGGAGGG | |
| | ACAACGGAGATGACAAGA | |
| | GGGACAGGATGGACCCCTG | |
| | GGCACATGATCGCAAACAC | |
| | CACCCCCGGCAACTGGACA | |
| | AGGCTGAGTTGGACGAGCG | |
| | ACCAGAAGGAGGGAGGCC | |
| | CCACCGGGAGAAGTACCCG | |
| | AGATCTGGGTCTCCCAACC | |
| | TGCCCCACTCTGTGTCCAGC | |
| | TACAAAAGCCGTGAAGACG | |
| | GCTACTACCGGAAAGAGCC | |
| | CAAAGCCAAGTGGGACAAG | |
| | TATCTGAAGCAGCAGCAGG | |
| | ATGCCCCCGGGAGGTCCAG | |
| | GAGGAAAGACGAGGCCAG | |
| | GCTGCGGGAAAGCAGACAC | |
| | CCCCATCCGGATGACTCAG | |
| | GCAAGGAAGATGGGCTGGG | |
| | GCCAAAGGTCACTAGGGCC | |
| | CCTGAGGGCGCCAAGGCCA | |
| | AGCAGAATGAGAAAAATA | |
| | AAACCAAGAGAACTGATAG | |
| | AGACCAAGAAGGAGCTGAT | |
| | GATAGAAAAGAAAACACA | |
| | ATGGCAGAGAATGAGGCTG | |
| | GAAAAGAGGAACAGGAGG | |
| | GCATGGAAGAAAGCCCTCA | |
| | ATCAGTGGGCAGACAGGAG | |
| | AAAGAAGCAGAGTTCTCTG | |
| | ATCCGGAAAACACAAGGAC | |
| | AAAGAAGGAACAAGATTG | |
| | GGAGAGTGAAAGTGAGGC | |
| | AGAGGGGGAGAGCTGGTAT | |
| | CCCACTAACATGGAGGAGC | |
| | TGGTGACAGTGGACGAGGT | |
| | TGGGGAAGAAGAAGATTTT | |
| | ATCGTGGAACCAGACATCC | |
| | CAGAGCTGGAAGAAATTGT | |
| | GCCCATTGACCAGAAAGAC | |
| | AAAATTTGCCCAGAAACAT | |
| | GTCTGTGTGTGACAACCAC | |
| | CTTAGACTTAGACCTGGCC | |
| | CAGGATTTCCCCAAGGAAG | |
| | GAGTCAAGGCCGTAGGGAA | |
| | TGGGGCTGCAGAAATCAGC | |
| | CTCAAGTCACCCAGAGAAC | |
| | TGCCCTCTGCTTCCACAAGC | |
| | TGTCCCAGTGACATGGACG | |
| | TGGAAATGCCTGGCCTAAA | |
| | TCTGGATGCTGAGCGGAAG | |
| | CCAGCTGAAAGTGAGACAG | |
| | GCCTCTCCCTGGAGGATTC | |
| | AGATTGCTACGAGAAGGAG | |
| | GCAAAGGGAGTGGAGAGCT | |
| | CAGATGTTCATCCAGCCCC | |
| | TACAGTCCAGCAAATGTCT | |
| | TCCCCTAAGCCAGCAGAGG | |
| | AGAGGGCCCGGCAGCCAAG | |
| | CCCATTTGTGGATGATTGC | |
| | AAGACCAGGGGGACCCCCG | |
| | AAGATGGGGCTTGTGAAGG | |
| | CAGCCCCCTGGAGGAGAAA | |
| | GCCAGCCCCCCCATCGAAA | |
| | CTGACCTCCAAAACCAAGC | |
| | CTGCCAAGAAGTGTTGACC | |
| | CCGGAAAACTCCAGGTACG | |
| | TGGAAATGAAATCTCTGGA | |
| | GGTGAGGTCACCAGAGTAC | |
| | ACTGAAGTGGAACTGAAAC | |
| | AGCCCCTTTCTTTGCCCTCT | |
| | TGGGAACCAGAGGATGTGT | |
| | TCAGTGAACTTAGCATTCCT | |
| | CTAGGGGTGGAGTTCGTGG | |
| | TTCCCAGGACTGGCTTTTAT | |
| | TGCAAGCTGTGTGGGCTGT | |
| | TCTACACGAGCGAGGAGAC | |
| | AGCAAAGATGAGCCACTGC | |
| | CGCAGCGCTGTCCACTACA | |
| | GGAACTTACAGAAATATTT | |
| | GTCCCAGCTGGCCGAGGAG | |
| | GGCCTCAAGGAGACCGAGG | |
| | GGGCAGATAGCCCGAGGCC | |
| | AGAGGACAGCGGAATCGTG | |
| | CCACGCTTCGAAAGGAAAA | |
| | AGCTCTGA | |
| Poly A | AATAAAAGATCCTTATTTTC | |
| | ATTGGATCTGTGTGTTGGTT | |
| | TTTTGTGTG | |
| ITR-R | AGGAACCCCTAGTGATGGA | |
| | GTTGGCCACTCCCTCTCTGC | |
| | GCGCTCGCTCGCTCACTGA | |
| | GGCCGGGCGACCAAAGGTC | |
| | GCCCGACGCCCGGGCTTTG | |
| | CCCGGGCGGCCTCAGTGAG | |
| | CGAGCGAGCGCGCAGAGA | |
| | GGGAGTGGCCAA | |

AAV production. Recombinant AAV (rAAV) particles comprising each of the constructs are made by suspension transfection of Expi293F cells with the pTR2-TNNT2-RBM20 constructs and other plasmids needed for rAAV production (e.g., comprising rep and cap expression cassettes) to generate three groups of rAAV comprising (1) AAV9 capsid proteins; (2) rh74 capsid proteins; and (3) rh74 variant capsid proteins comprising a tryptophan to arginine mutation at amino acid 505 of the rh74 VP1 capsid protein. Vector is isolated using a capture column followed by an anion exchange column and purified using a cesium chloride gradient to a titer of 2E+13 to 5E+13 vg/ml.

### Example 1. In Vitro Expression Study

An rAAV particle comprising the RBM20 constructs is made as described above and delivered to HEK293 cells, C2C12 myoblast cells, or cardiomyocytes derived from human induced pluripotent stem cells. Whole cell lysates are generated and probed for expression of RBM20 by ELISA and/or immunoblotting.

### Example 2. In Vivo Expression Study

The rAAV comprising the RBM20 construct is made as described above and administered via the facial vein to newborn C57BL/6 mice (n=6-10/group) at 5E+13 vector genomes per kg subject (vg/kg). Two to four weeks after rAAV dosing, heart, diaphragm and skeletal muscle tissues from mice subjects are harvested and whole cell lysates are analyzed for RBM20 expression using ELISA and/or immunoblot.

The rAAV comprising the RBM20 constructs is made as described above and administered via the jugular vein to 5-7 weeks old C57BL/6 mice (n=6-10/group) at three different doses: 1E+13 vg/kg, 5E+13 vg/kg or 1+E14 vg/kg. One month after rAAV dosing, heart, diaphragm and skeletal muscle tissues are harvested and whole cell lysates are analyzed for RBM20 expression using ELISA and/or immunoblot.

### Example 3. Restoration of RBM20 Expression In Vivo

Mutations in RBM20, encoding RNA binding motif protein 20 (RBM20), are known to be causative of DCM. RBM20 is a major regulator of heart-specific alternative splicing of the TTN gene, which is found to be most frequently mutated in patients with idiopathic DCM (approximately 20-25%). The TTN gene has the largest number of exons (364 in humans) and titin, a sarcomeric protein encoded by the TTN gene, is the largest known protein in mammals. In an RBM20 mutant rat strain lacking nearly all the RBM20 exons, the shortest cardiac titin isoform N2B is not expressed. Therefore, RBM20 is a key regulator of TTN pre-mRNA processing in the heart and cause DCM phenotypes through altered splicing of the RBM20-regulated genes. Missensc mutations in a highly conserved RSRSP stretch, within an arginine/serine (RS)-rich region and not in the RNA binding domains are the most frequent disease alleles.

RBM20 S637A/S637A and RBM20 KO/KO mice lose RBM20-dependent alternative splicing and are suitable to testing rAAV-RMB20 gain of function.

The RBM20^{S637A} knock-in mouse model carries the orthologous mouse mutation for the human S637A mutation. The disease phenotype can be mimicked with overexpression of the S637A allele via a vector-based transgenic or in a homozygous knock-in mouse model.

rAAV comprising the RBM20 construct is made as described above and delivered via a single IV injection to presymptomatic and/or symptomatic RBM20 mutant mice using different doses. Exemplary doses include 1E+13 vg/kg, 5E+13 vg/kg, and 1+E14 vg/kg. Endpoints include survival as well as cardiac function monitored by echocardiography. Upon necropsy, heart tissues are collected and whole tissue lysates are analyzed for AAV biodistribution by digital droplet PCR (ddPCR) and for human RBM20 expression by ELISA and/or immunoblot. In addition, tissue sections are analyzed for histopathology. Therapeutic effects of the rAAV are assessed via the measured endpoints and/or histopathology assessments.

### Non-Limiting Embodiments

The following numerated Embodiments represent non-limiting aspects of the invention:

1. A nucleic acid comprising an expression cassette comprising a human RBM20 coding sequence, a silencing element, wherein the coding sequence and the silencing element are each operably linked to a promoter and optionally an enhancer element, and wherein the expression cassette is flanked on each side by an inverted terminal repeat sequence.

2. The nucleic acid of Embodiment 1, wherein the human RBM20 coding sequence is codon-optimized for expression in human cells.

3. The nucleic acid of Embodiment 1 or 2, wherein the human RBM20 coding sequence comprises a nucleic acid sequence having at least about 85% sequence identity to the sequence of SEQ ID NO: 5; optionally wherein the human RBM20 coding sequence comprises the sequence of SEQ ID NO: 5.

4. The nucleic acid of any one of Embodiments 1-3, wherein the promoter comprises a cardiac specific promoter.

5. The nucleic acid of Embodiment 4, wherein the promoter is selected from the group consisting of: TNNT2, MHCK9, and combinations thereof.

6. The nucleic acid of Embodiment 4 or 5, wherein the promoter comprises a nucleic acid sequence having at least about 85% sequence identity to the sequence of SEQ ID NO: 2 or 16.

7. The nucleic acid of any one of Embodiments 4-6, wherein the promoter comprises a nucleic acid sequence comprising the sequence of SEQ ID NO: 2 or 16.

8. The nucleic acid of any one of Embodiments 1-7, wherein the expression cassette has at least about 85% sequence identity to the sequence of SEQ ID NOs: 1-7 or SEQ ID NOs: 13-21, arranged in sequence.

9. The nucleic acid of Embodiment 8, wherein the expression cassette comprises the sequence of SEQ ID NO: 1-7 or SEQ ID NOs: 13-21, arranged in sequence.

10. The nucleic acid of any one of Embodiments 1-9, wherein the nucleic acid is a recombinant adeno-associated virus (rAAV) vector.

11. The nucleic acid of Embodiment 10, wherein the nucleic acid is a single-stranded nucleic acid vector.

12. A recombinant adeno-associated virus (rAAV) particle comprising the nucleic acid of any one of embodiments 1-11.

13. The rAAV particle of Embodiment 12, wherein the rAAV particle is an AAV9 particle.

14. The rAAV particle of Embodiment 12, wherein the rAAV particle is an AAVrh74 particle.

15. The rAAV particle of Embodiment 12, wherein the rAAV particle is an AAVrh10 particle.

16. A composition comprising a plurality of the rAAV particle of any one of Embodiments 12-15.

17. The composition of Embodiment 16 further comprising a pharmaceutically acceptable carrier.

18. A method of treating dilated cardiomyopathy, the method comprising:
administering a therapeutically effective amount of rAAV comprising a nucleic acid expression construct comprising a human RBM20 coding sequence, silencing element,
wherein the coding sequence and the silencing element are operably linked to a promoter and optionally an enhancer element, wherein the expression construct is flanked on each side by an inverted terminal repeat sequence, and wherein said administration results in expression of a therapeutically effective amount of human TBM20 thereby treating the dilated cardiomyopathy.

19. The method of Embodiment 18, wherein the rAAV is administered via intravenous injection.

20. The method of Embodiment 18, wherein between about 1×10¹³ and about 1×10¹⁴ rAAV vector genomes are administered.

21. A method of increasing expression of human RBM20 in a target cell, comprising:
contacting a target cell with a plurality of rAAV particles comprising a nucleic acid expression cassette comprising a functional human RBM20 coding sequence, a silencing element, wherein the coding sequence and the silencing element are operably linked to a promoter and optionally an enhancer element, wherein the expression construct is flanked on each side by an inverted terminal repeat sequence, and wherein the step of contacting results in increased expression of functional human RBM20 in the target cell as compared to prior to the contacting, thereby increasing the expression of functional human RBM20.

22. The method of Embodiment 12, wherein the contacting is in vivo.

23. The method of Embodiment 21 or 22, for the treatment of dilated cardiomyopathy.

24. Use of the nucleic acid of any one of Embodiments 1-11, the rAAV particle of any one of Embodiments 12-15, or the composition of Embodiment 16 or 17, in the manufacture of a medicament for the treatment of dilated cardiomyopathy.

25. Use of the nucleic acid of any one of Embodiments 1-11, the rAAV particle of any one of Embodiments 12-15, or the composition of Embodiment 16 or 17, for the treatment of dilated cardiomyopathy.

26. The nucleic acid of any one of Embodiments 1-11, wherein the silencing element encodes an shRNA sequence.

27. A nucleic acid comprising an expression cassette comprising a human RBM20 coding sequence operably linked to a promoter and optionally an enhancer element, wherein the expression cassette is flanked on each side by an inverted terminal repeat sequence.

28. A method of treating dilated cardiomyopathy, the method comprising:
administering a therapeutically effective amount of rAAV comprising a nucleic acid expression construct comprising a human RBM20 coding sequence operably linked to a promoter and optionally an enhancer element, wherein the expression construct is flanked on each side by an inverted terminal repeat sequence, and wherein the step of administering results in expression of a therapeutically effective amount of human RBM20, thereby treating the dilated cardiomyopathy.

29. The method of embodiment 28, further comprising administering a therapeutically effective amount of a silencing construct.

30. A method of treating dilated cardiomyopathy, the method comprising:
administering a therapeutically effective amount of rAAV comprising a nucleic acid expression construct comprising a human RBM20 coding sequence operably linked to a promoter and optionally an enhancer element, wherein the expression construct is flanked on each side by an inverted terminal repeat sequence, and wherein the step of administering results in expression of a therapeutically effective amount of human RBM20 thereby treating the dilated cardiomyopathy.

31. The method of embodiment 30, further comprising administering a therapeutically effective amount of a silencing construct.

32. A nucleic acid comprising an expression cassette comprising a human RBM20 coding sequence, a silencing element, wherein the coding sequence and the silencing element are each operably linked to a promoter and optionally an enhancer element, and wherein the expression cassette is flanked on each side by an inverted terminal repeat sequence.

33. The nucleic acid of Embodiment 32, wherein the human RBM20 coding sequence is codon-optimized for expression in human cells.

34. The nucleic acid of Embodiment 32 or Embodiment 33, wherein the human RBM20 coding sequence comprises a nucleic acid sequence having at least about 85% sequence identity to the sequence of SEQ ID NO: 5.

35. The nucleic acid of any one of Embodiments 32-34, wherein the promoter comprises a cardiac specific promoter.

36. The nucleic acid of Embodiment 35, wherein the promoter is selected from the group consisting of: TNNT2, MHCK9, and combinations thereof.

37. The nucleic acid of Embodiment 35 or 36, wherein the promoter sequence comprises a nucleic acid sequence having has at least about 85% sequence identity to the sequence of SEQ ID NO: 2 or 16.

38. The nucleic acid of any one of Embodiments 32 to 37, wherein the expression cassette has at least about 85% sequence identity to the sequence of SEQ ID NOs: 1-7 or SEQ ID NOs: 13-21, arranged in sequence.

39. The nucleic acid of Embodiment 38, wherein the expression cassette comprises the sequence of SEQ ID NO: 1-7 or SEQ ID NOs: 13-21, arranged in sequence.

40. The nucleic acid of any one of Embodiments 32 to 39, wherein the nucleic acid is a recombinant adeno-associated virus (rAAV) vector.

41. The nucleic acid of Embodiment 40, wherein the nucleic acid is a single-stranded nucleic acid vector.

42. A recombinant adeno-associated virus (rAAV) particle comprising the nucleic acid of Embodiment 40 or Embodiment 41.

43. The rAAV particle of Embodiment 42, wherein the rAAV particle is an AAV9 particle.

44. The rAAV particle of Embodiment 42, wherein the rAAV particle is an AAVrh74 particle.

45. The rAAV particle of Embodiment 42, wherein the rAAV particle is an AAVrh10 particle.

46. A composition comprising a plurality of the rAAV particle of Embodiment 10, wherein the rAAV is selected from one or more of: AAV9 particles, AAVrh74 particles, and AAVrh10 particles.

47. The composition of Embodiment 46, further comprising a pharmaceutically acceptable carrier.

48. A method of inducing increasing expression of human RBM20 in a target cell, comprising: contacting a target cell with a plurality of rAAV particles comprising a nucleic acid expression cassette comprising a functional human RBM20 coding sequence, a silencing element, wherein the coding sequence and the silencing element are each operably linked to a promoter and optionally an enhancer element, wherein the expression construct is flanked on each side by an inverted terminal repeat sequence, and wherein the step of contacting results in the target cell increasing expression of functional human RBM20 in the target cell as compared to prior to the contacting, thereby increasing the expression of functional human RBM20.

49. The method of Embodiment 48, wherein the contacting is in vivo.

50. The method of Embodiment 48 or 49, for the treatment of dilated cardiomyopathy.

51. Use of the nucleic acid of any one of Embodiments 32 to 41, the rAAV particle of any one of Embodiments 42-45, or the composition of Embodiment 46 or 47, in the manufacture of a medicament for the treatment of dilated cardiomyopathy.

52. Use of the nucleic acid of any one of Embodiments 32 to 41, the rAAV particle of any one of Embodiments 42-45, or the composition of Embodiment 46 or 47, for the treatment of dilated cardiomyopathy.

53. The nucleic acid of any one of Embodiments 32-41, wherein the silencing element encodes an shRNA sequence.

54. A nucleic acid comprising an expression construct comprising:
a human RBM20 coding sequence; a cardiac enhancer element operable linked to a promoter; and a Kozak sequence, wherein the Kozak sequence enhances transgene expression in the heart, wherein the expression construct is flanked on each side by an inverted terminal repeat sequence, wherein the Kozak sequence is non-native with respect to the human RBM20 coding sequence, the cardiac enhancer element, and/or the promoter.

55. The nucleic acid of Embodiment 54, wherein the Kozak sequence is a synthetic sequence and has at least 85% sequence identity to the sequence of SEQ ID NO: 47.

56. The nucleic acid of Embodiment 54, wherein the Kozak sequence is a synthetic sequence and has at least 85% sequence identity to the sequence of SEQ ID NO: 36.

What is claimed is:

1. A nucleic acid comprising an expression cassette comprising a human RBM20 coding sequence, a silencing element, wherein the coding sequence and the silencing element are each operably linked to a promoter and optionally an enhancer element, and wherein the expression cassette is flanked on each side by an inverted terminal repeat sequence.

2. The nucleic acid of claim 1, wherein the human RBM20 coding sequence is codon-optimized for expression in human cells.

3. The nucleic acid of claim 1, wherein the human RBM20 coding sequence has a nucleic acid sequence having at least about 85% sequence identity to the sequence of SEQ ID NO: 5.

4. The nucleic acid of claim 1, wherein the promoter comprises a cardiac specific promoter.

5. The nucleic acid of claim 4, wherein the promoter is selected from the group consisting of: TNNT2, MHCK9, and combinations thereof.

6. The nucleic acid of claim 4, wherein the promoter comprises a nucleic acid sequence having at least about 85% sequence identity to the sequence of SEQ ID NO: 2 or 16.

7. The nucleic acid of claim 4, wherein the promoter comprises a nucleic acid sequence comprising the sequence of SEQ ID NO: 2 or 16.

8. The nucleic acid of claim 1, wherein the expression cassette has at least about 85% sequence identity to the sequence of SEQ ID NOs: 1-7 or ID NOs: 13-21, arranged in sequence.

9. The nucleic acid of claim 8, wherein the expression cassette comprises the sequence of SEQ ID NO: 1-7 or ID NOs: 13-21, arranged in sequence.

10. The nucleic acid of claim 1, wherein the nucleic acid is a recombinant adeno-associated virus (rAAV) vector.

11. The nucleic acid of claim 10, wherein the nucleic acid is a single-stranded nucleic acid vector.

12. A recombinant adeno-associated virus (rAAV) particle comprising the nucleic acid of any one of claims 1-11.

13. The rAAV particle of claim 12, wherein the rAAV particle is an AAV9 particle.

14. The rAAV particle of claim 12, wherein the rAAV particle is an AAVrh74 particle.

15. The rAAV particle of claim 12, wherein the rAAV particle is an AAVrh10 particle.

16. A composition comprising a plurality of the rAAV particle of any one of claims 12-15.

17. The composition of claim 16, further comprising a pharmaceutically acceptable carrier.

18. A method of treating dilated cardiomyopathy, the method comprising:
administering a therapeutically effective amount of rAAV comprising a nucleic acid expression construct comprising a human RBM20 coding sequence, silencing element, wherein the coding sequence and the silencing element are operably linked to a promoter and optionally an enhancer element, wherein the expression construct is flanked on each side by an inverted terminal repeat sequence, and wherein said administration results in expression of a therapeutically effective amount of human TBM20 thereby treating the dilated cardiomyopathy.

19. The method of claim 18, wherein the rAAV is administered via intravenous injection.

20. The method of claim 18, wherein between about 1×10¹³ and about 1×10¹⁴ rAAV vector genomes are administered.

21. A method of inducing increased expression of human RBM20 in a target cell, comprising:
contacting a target cell with a plurality of rAAV particles comprising a nucleic acid expression cassette comprising a functional human RBM20 coding sequence, a silencing element, wherein the coding sequence and the silencing element are operably linked to a promoter and optionally an enhancer element, wherein the expression construct is flanked on each side by an inverted terminal repeat sequence, and
wherein the step of contacting results in the target cell increased expression of functional human RBM20 in the target cell as compared to prior to the contacting, thereby increasing the expression of functional human RBM20.

22. The method of claim 21, wherein the contacting is in vivo.

23. The method of claim 21 or 22, for the treatment of dilated cardiomyopathy.

24. Use of the nucleic acid of any one of claims 1-11, the rAAV particle of any one of claims 12-14, or the composition of claim 16 or 17, in the manufacture of a medicament for the treatment of dilated cardiomyopathy.

25. Use of the nucleic acid of any one of claims 1-11, the rAAV particle of any one of claims 12-15, or the composition of claim 16 or 17, for the treatment of dilated cardiomyopathy.

26. The nucleic acid of any one of claims 1-11, wherein the silencing element encodes an shRNA sequence.

27. A nucleic acid comprising an expression cassette comprising a human RBM20 coding sequence operably linked to a promoter and optionally an enhancer element, wherein the expression cassette is flanked on each side by an inverted terminal repeat sequence.

28. A method of treating dilated cardiomyopathy, the method comprising:
administering a therapeutically effective amount of rAAV comprising a nucleic acid expression construct comprising a human RBM20 coding sequence operably linked to a promoter and optionally an enhancer element, wherein the expression construct is flanked on each side by an inverted terminal repeat sequence, and wherein the step of administering results in expression of a therapeutically effective amount of human RBM20, thereby treating the dilated cardiomyopathy.

29. The method of claim 28, further comprising administering a therapeutically effective amount of a silencing construct.

30. A method of treating dilated cardiomyopathy, the method comprising:
administering a therapeutically effective amount of rAAV comprising a nucleic acid expression construct comprising a human RBM20 coding sequence operably linked to a promoter and optionally an enhancer element,
wherein the expression construct is flanked on each side by an inverted terminal repeat sequence, and wherein said step of administering results in expression of a therapeutically effective amount of human RBM20 thereby treating the dilated cardiomyopathy.

31. The method of claim 30, further comprising administering a therapeutically effective amount of a silencing construct.

32. A nucleic acid comprising an expression cassette comprising a human RBM20 coding sequence, a silencing element, wherein the coding sequence and the silencing element are each operably linked to a promoter and optionally an enhancer element, and wherein the expression cassette is flanked on each side by an inverted terminal repeat sequence.

33. The nucleic acid of claim 32, wherein the human RBM20 coding sequence is codon-optimized for expression in human cells.

34. The nucleic acid of claim 32 or claim 33, wherein the human RBM20 coding sequence comprises a nucleic acid sequence having at least about 85% sequence identity to the sequence of SEQ ID NO: 5.

35. The nucleic acid of any one of claims 32-34, wherein the promoter comprises a cardiac specific promoter.

36. The nucleic acid of claim 35, wherein the promoter is selected from the group consisting of: TNNT2, MHCK9, and combinations thereof.

37. The nucleic acid of claim 35 or 36, wherein the promoter sequence comprises a nucleic acid sequence having has at least about 85% sequence identity to the sequence of SEQ ID NO: 2 or 16.

38. The nucleic acid of any one of claims 32 to 37, wherein the expression cassette has at least about 85% sequence identity to the sequence of SEQ ID NOs: 1-7 or ID NOs: 13-21, arranged in sequence.

39. The nucleic acid of claim 38, wherein the expression cassette comprises the sequence of SEQ ID NO: 1-7 or ID NOs: 13-21, arranged in sequence.

40. The nucleic acid of any one of claims 32 to 39, wherein the nucleic acid is a recombinant adeno-associated virus (rAAV) vector.

41. The nucleic acid of claim 40, wherein the nucleic acid is a single-stranded nucleic acid vector.

42. A recombinant adeno-associated virus (rAAV) particle comprising the nucleic acid of claim 40 or claim 41.

43. The rAAV particle of claim 42, wherein the rAAV particle is an AAV9 particle.

44. The rAAV particle of claim 42, wherein the rAAV particle is an AAVrh74 particle.

45. The rAAV particle of claim 42, wherein the rAAV particle is an AAVrh10 particle.

46. A composition comprising a plurality of the rAAV particle of claim 10, wherein the rAAV is selected from one or more of: AAV9 particles, AAVrh74 particles, and AAVrh10 particles.

47. The composition of claim 46, further comprising a pharmaceutically acceptable carrier.

48. A method of inducing increasing expression of human RBM20 in a target cell, comprising:
contacting a target cell with a plurality of rAAV particles comprising a nucleic acid expression cassette comprising a functional human RBM20 coding sequence, a silencing element, wherein the coding sequence and the silencing element are each operably linked to a promoter and optionally an enhancer element, wherein the expression construct is flanked on each side by an inverted terminal repeat sequence, and
wherein said step of contacting results in the target cell increasing expression of functional human RBM20 in the target cell as compared to prior to the contacting, thereby increasing the expression of functional human RBM20.

49. The method of claim 48, wherein the contacting is in vivo.

50. The method of claim 48 or 49, for the treatment of dilated cardiomyopathy.

51. Use of the nucleic acid of any one of claims 32 to 41, the rAAV particle of any one of claims 42-45, or the composition of claim 46 or 47, in the manufacture of a medicament for the treatment of dilated cardiomyopathy.

52. Use of the nucleic acid of any one of claims 32 to 41, the rAAV particle of any one of claims 42-45, or the composition of claim 46 or 47, for the treatment of dilated cardiomyopathy.

53. The nucleic acid of any one of claims 32-41, wherein the silencing element encodes an shRNA sequence.

54. A nucleic acid comprising an expression construct comprising:
a human RBM20 coding sequence;
a cardiac enhancer element operable linked to a promoter; and
a Kozak sequence, wherein the Kozak sequence enhances transgene expression in the heart, wherein the expression construct is flanked on each side by an inverted terminal repeat sequence, wherein the Kozak sequence is non-native with respect to the human RBM20 coding sequence, the cardiac enhancer element, and/or the promoter.

55. The nucleic acid of claim 54, wherein the Kozak sequence is a synthetic sequence and has at least 85% sequence identity to the sequence of SEQ ID NO: 47.

56. The nucleic acid of claim 54, wherein the Kozak sequence is a synthetic sequence and has at least 85% sequence identity to the sequence of SEQ ID NO: 36.
